SEQUENCE LISTING (38 sequences)

SEQ ID NO: 1 - DNA, 1056 bp, Mus musculus
  source 1..1056: mol_type = unassigned DNA
atggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaac

SEQ ID NO: 2 - protein, 352 aa, Mus musculus
MAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSN

SEQ ID NO: 3 - DNA, 957 bp, Mus musculus
  source 1..957: mol_type = unassigned DNA
atgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatg

SEQ ID NO: 4 - protein, 319 aa, Mus musculus
MYNMMETELKPPGPQQASGGGGGGGNATAAATGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQEQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMAQHYQSGPVPGTAINGTLPLSHM

SEQ ID NO: 5 - DNA, 1446 bp, Mus musculus
  source 1..1446: mol_type = unassigned DNA
atgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccagcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgcgacagggccttttccaggtcggaccaccttgccttacacatgaagaggcac

SEQ ID NO: 6 - protein, 482 aa, Mus musculus
MRQPPGESDMAVSDALLPSFSTFASGPAGREKTLRPAGAPTNRWREELSHMKRLPPLPGRPYDLAATVATDLESGGAGAACSSNNPALLARRETEEFNDLLDLDFILSNSLTHQESVAATVTTSASASSSSSPASSGPASAPSTCSFSYPIRAGGDPGVAASNTGGGLLYSRESAPPPTAPFNLADINDVSPSGGFVAELLRPELDPVYIPPQQPQPPGGGLMGKFVLKASLTTPGSEYSSPSVISVSKGSPDGSHPVVVAPYSGGPPRMCPKIKQEAVPSCTVSRSLEAHLSAGPQLSNGHRPNTHDFPLGRQLPTRTTPTLSPEELLNSRDCHPGLPLPPGFHPHPGPNYPPFLPDQMQSQVPSLHYQELMPPGSCLPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRH

SEQ ID NO: 7 - DNA, 376 bp, synthetic construct
  misc_feature 1..376: note = Synthetic Polynucleotide
  source 1..376: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 8 - DNA, 169 bp, synthetic construct
  misc_feature 1..169: note = Synthetic Polynucleotide
  source 1..169: mol_type = other DNA
tgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccg

SEQ ID NO: 9 - protein, 21 aa, synthetic construct
  REGION 1..21: note = Synthetic Polypeptide
GSGEGRGSLLTCGDVEENPGP

SEQ ID NO: 10 - DNA, 708 bp, synthetic construct
  misc_feature 1..708: note = Synthetic Polynucleotide
  source 1..708: mol_type = other DNA
atgtctaggctggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgattttgaccttgacatgctccccgggtaa

SEQ ID NO: 11 - protein, 235 aa, synthetic construct
  REGION 1..235: note = Synthetic Polypeptide
MSRLDKSKVINGALELLNGVGIEGLTTRKLAQKLGVEQPTLYWHVKNKRALLDALPIEMLDRHHTHFCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPTDALDDFDLDMLPADALDDFDLDMLPG

SEQ ID NO: 12 - DNA, 747 bp, synthetic construct
  misc_feature 1..747: note = Synthetic Polynucleotide
  source 1..747: mol_type = other DNA
atgtcccgcttggataagagcaaggtaataaatagcgcactcgaactcctcaacggcgtgggcatcgaaggtctgactactcgaaagctcgcccagaaattgggtgtggagcaacctacattgtattggcatgtcaagaacaaaagagccctgctggacgctcttcctattgaaatgcttgacaggcatcacactcattcctgcccccttgaggtcgagagttggcaagattttctccgaaacaatgcaaagtcctaccgctgcgcacttttgtcccatagggatggagcaaaagtgcacctgggaaccaggccaacagagaaacaatacgagactctcgagaaccagttggctttcttgtgccaacaggggttctcacttgaaaatgccctttacgcactgtcagccgttggacattttaccctggggtgcgttcttgaggagcaagaacatcaggttgctaaggaggagcgcgagactccaaccactgattctatgccacctttgctgaaacaggccattgaacttttcgatagacagggtgctgaacctgcctttctcttcgggttggagctgattatttgtggtctcgaaaaacagctgaaatgtgaaagtggtggccctactgacgccctcgatgatttcgacctggatatgctgccagccgatgcacttgatgatttcgatttggatatgcttccagccgacgcactggacgacttcgatttggacatgcttcccggttaa

SEQ ID NO: 13 - protein, 248 aa, synthetic construct
  REGION 1..248: note = Synthetic Polypeptide
MSRLDKSKVINSALELLNGVGIEGLTTRKLAQKLGVEQPTLYWHVKNKRALLDALPIEMLDRHHTHSCPLEVESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 14 - DNA, 1050 bp, synthetic construct
  misc_feature 1..1050: note = Synthetic Polynucleotide
  source 1..1050: mol_type = other DNA
atgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctga

SEQ ID NO: 15 - protein, 349 aa, synthetic construct
  REGION 1..349: note = Synthetic Polypeptide
MPLYHAIASRMAFIFSSLYKSWLLSLYEELWPVVRQRGVVCTVFADATPTGWGIATTCQLLSGTFAFPLPIATAELIAACLARCWTGARLLGTDNSVVLSGKSSSFPWLLACVATWILRGTSFCYVPSALNPADLPSRGLLPALRPLPRLRLRPQTSRISLWAASPHRYRRPRDLEKHGAITSSNTAATNADCAWLEAQEEEEVGFPVTPQVPLRPMTYKAAVDLSHFLKEKGGLEGLIHSQRRQDILDLWIYHTQGYFPDWQNYTPGPGIRYPLTFGWCYKLVPVEQEKVEEANEGENTRLLHPVSLHGMDDPEREVLEWRFDSRLAFHHMARELHPDCTGSLWLDQI

SEQ ID NO: 16 - DNA, 7408 bp, synthetic construct
  misc_feature 1..7408: note = Synthetic Polynucleotide
  source 1..7408: mol_type = other DNA
ttatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagcggccgcgccaccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaacgctagcggcagcggcgccacgaacttctctctgttaaagcaagcaggagatgttgaagaaaaccccgggcctgcatgcatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatggcatgcggctccggcgagggcaggggaagtcttctaacatgcggggacgtggaggaaaatcccggcccactcgagatgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccagcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgcgacagggccttttccaggtcggaccaccttgccttacacatgaagaggcactaaatgactagtgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaa

SEQ ID NO: 17 - DNA, 5657 bp, synthetic construct
  misc_feature 1..5657: note = Synthetic Polynucleotide
  source 1..5657: mol_type = other DNA
ttatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcctgatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcggccgcattaaacgccaccatgtcccgcttggataagagcaaggtaataaatagcgcactcgaactcctcaacggcgtgggcatcgaaggtctgactactcgaaagctcgcccagaaattgggtgtggagcaacctacattgtattggcatgtcaagaacaaaagagccctgctggacgctcttcctattgaaatgcttgacaggcatcacactcattcctgcccccttgaggtcgagagttggcaagattttctccgaaacaatgcaaagtcctaccgctgcgcacttttgtcccatagggatggagcaaaagtgcacctgggaaccaggccaacagagaaacaatacgagactctcgagaaccagttggctttcttgtgccaacaggggttctcacttgaaaatgccctttacgcactgtcagccgttggacattttaccctggggtgcgttcttgaggagcaagaacatcaggttgctaaggaggagcgcgagactccaaccactgattctatgccacctttgctgaaacaggccattgaacttttcgatagacagggtgctgaacctgcctttctcttcgggttggagctgattatttgtggtctcgaaaaacagctgaaatgtgaaagtggtggccctactgacgccctcgatgatttcgacctggatatgctgccagccgatgcacttgatgatttcgatttggatatgcttccagccgacgcactggacgacttcgatttggacatgcttcccggttaaactagtctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgggggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaa

SEQ ID NO: 18 - DNA, 1215 bp, synthetic construct
  misc_feature 1..1215: note = Synthetic Polynucleotide
  source 1..1215: mol_type = other DNA
gatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 19 - DNA, 19 bp, synthetic construct
  misc_feature 1..19: note = Synthetic Polynucleotide
  source 1..19: mol_type = other DNA
tccctatcagtgatagaga

SEQ ID NO: 20 - DNA, 68 bp, synthetic construct
  misc_feature 1..68: note = Synthetic Polynucleotide
  source 1..68: mol_type = other DNA
gctttaggcgtgtacggtgggcgcctataaaagcagagctcgtttagtgaaccgtcagatcgcctgga

SEQ ID NO: 21 - DNA, 248 bp, synthetic construct
  misc_feature 1..248: note = Synthetic Polynucleotide
  source 1..248: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtt

SEQ ID NO: 22 - DNA, 141 bp, synthetic construct
  misc_feature 1..141: note = Synthetic Polynucleotide
  source 1..141: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 23 - DNA, 438 bp, synthetic construct
  misc_feature 1..438: note = Synthetic Polynucleotide
  source 1..438: mol_type = other DNA
aattcgtacacgcctacctcgacccatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagctcgtttagtgaaccgtcagatcgcctggagacgccatccacgctgttttgacctccatagaagacaccgggaccgatccagcctggatcgc

SEQ ID NO: 24 - DNA, 315 bp, synthetic construct
  misc_feature 1..315: note = Synthetic Polynucleotide
  source 1..315: mol_type = other DNA
gagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcc

SEQ ID NO: 25 - DNA, 618 bp, synthetic construct
  misc_feature 1..618: note = Synthetic Polynucleotide
  source 1..618: mol_type = other DNA
atggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtggg

SEQ ID NO: 26 - protein, 206 aa, synthetic construct
  REGION 1..206: note = Synthetic Polypeptide
MARLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESG

SEQ ID NO: 27 - DNA, 1008 bp, synthetic construct
  misc_feature 1..1008: note = Synthetic Polynucleotide
  source 1..1008: mol_type = other DNA
atggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagtttaa

SEQ ID NO: 28 - protein, 335 aa, synthetic construct
  REGION 1..335: note = Synthetic Polypeptide
MARLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSPKKKRKVDGGGALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 29 - DNA, 977 bp, synthetic construct
  misc_feature 1..977: note = Synthetic Polynucleotide
  source 1..977: mol_type = other DNA
accttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgagggagacaggggcgcaggagtcaggcaaaggcgatgcgggggtgcgactacacgcagttggaaacagtcgtcagaagattctggaaactatcttgctggctataaacttgagggaagcagaaggccaacattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctacaaatagtgcagacagctaaggggctccgtcacccatcttcacatccactccagccggctgcccgcccgctgcctcctctgtgcgtccgcccagccagcctcgtccacgcc

SEQ ID NO: 30 - DNA, 5428 bp, synthetic construct
  misc_feature 1..5428: note = Synthetic Polynucleotide
  source 1..5428: mol_type = other DNA
ttatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcctagatctaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgagggagacaggggcgcaggagtcaggcaaaggcgatgcgggggtgcgactacacgcagttggaaacagtcgtcagaagattctggaaactatcttgctggctataaacttgagggaagcagaaggccaacattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctacaaatagtgcagacagctaaggggctccgtcacccatcttcacatccactccagccggctgcccgcccgctgcctcctctgtgcgtccgcccagccagcctcgtccacgccaagcttgcggccgcattaaacgccaccatgtcccgcttggataagagcaaggtaataaatagcgcactcgaactcctcaacggcgtgggcatcgaaggtctgactactcgaaagctcgcccagaaattgggtgtggagcaacctacattgtattggcatgtcaagaacaaaagagccctgctggacgctcttcctattgaaatgcttgacaggcatcacactcattcctgcccccttgaggtcgagagttggcaagattttctccgaaacaatgcaaagtcctaccgctgcgcacttttgtcccatagggatggagcaaaagtgcacctgggaaccaggccaacagagaaacaatacgagactctcgagaaccagttggctttcttgtgccaacaggggttctcacttgaaaatgccctttacgcactgtcagccgttggacattttaccctggggtgcgttcttgaggagcaagaacatcaggttgctaaggaggagcgcgagactccaaccactgattctatgccacctttgctgaaacaggccattgaacttttcgatagacagggtgctgaacctgcctttctcttcgggttggagctgattatttgtggtctcgaaaaacagctgaaatgtgaaagtggtggccctactgacgccctcgatgatttcgacctggatatgctgccagccgatgcacttgatgatttcgatttggatatgcttccagccgacgcactggacgacttcgatttggacatgcttcccggttaaactagtctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgggggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaa

SEQ ID NO: 31 - DNA, 5332 bp, synthetic construct
  misc_feature 1..5332: note = Synthetic Polynucleotide
  source 1..5332: mol_type = other DNA
aaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgatctcaattcggccattacggccggatccggctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcaccatgtctagactggacaagagcaaaatcataaacagcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacagctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaagcaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaactaagtaaggatcatcttaattaaatcgataaggatctggccgcctcggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaactagcgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccata

SEQ ID NO: 32 - DNA, 5392 bp, synthetic construct
  misc_feature 1..5392: note = Synthetic Polynucleotide
  source 1..5392: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggatgaggatcctctagagtcgacctgcagaagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 33 - DNA, 7732 bp, synthetic construct
  misc_feature 1..7732: note = Synthetic Polynucleotide
  source 1..7732: mol_type = other DNA
ttatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgtacacgcctacctcgacccatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagctcgtttagtgaaccgtcagatcgcctggagacgccatccacgctgttttgacctccatagaagacaccgggaccgatccagcctggatcgcggccgcgccaccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaacgctagcggcagcggcgccacgaacttctctctgttaaagcaagcaggagatgttgaagaaaaccccgggcctgcatgcatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatggcatgcggctccggcgagggcaggggaagtcttctaacatgcggggacgtggaggaaaatcccggcccactcgagatgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccagcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgcgacagggccttttccaggtcggaccaccttgccttacacatgaagaggcactaaatgactagtctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgggggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaa

SEQ ID NO: 34 - DNA, 186 bp, synthetic construct
  misc_feature 1..186: note = Synthetic Polynucleotide
  source 1..186: mol_type = other DNA
gaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctaga

SEQ ID NO: 35 - DNA, 783 bp, synthetic construct
  misc_feature 1..783: note = Synthetic Polynucleotide
  source 1..783: mol_type = other DNA
agccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctg

SEQ ID NO: 36 - DNA, 570 bp, synthetic construct
  misc_feature 1..570: note = Synthetic Polynucleotide
  source 1..570: mol_type = other DNA
cgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 37 - DNA, 1416 bp, synthetic construct
  misc_feature 1..1416: note = Synthetic Polynucleotide
  source 1..1416: mol_type = other DNA
gcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 38 - DNA, 5657 bp, synthetic construct
  misc_feature 1..5657: note = Synthetic Polynucleotide
  source 1..5657: mol_type = other DNA
ttctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccactattgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaagatgccacctgaaattataaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaggttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatccttatcgggaatttggatcccccgctttaaaaaacctcccacatctccccctgaacctgaaacataaaatgaatgcaattgttgttgttaacttgtttattgcagcttataatggttacaaataaagcaatagcatcacaaatttcacaaataaagctagaatggttacaaataaagcaatagcatcacaaatttcacaaataaacaccacggaattgtcagtgcccaacagccgagcccctgtccagcagcgggcaaggcaggcggcgatgagttccgccgtggcaagaactaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattgctagactagtttaaccgggaagcatgtccaaatcgaagtcgtccagtgcgtcggctggaagcatatccaaatcgaaatcatcaagtgcatcggctggcagcatatccaggtcgaaatcatcgagggcgtcagtagggccaccactttcacatttcagctgtttttcgagaccacaaataatcagctccaacccgaagagaaaggcaggttcagcaccctgtctatcgaaaagttcaatggcctgtttcagcaaaggtggcatagaatcagtggttggagtctcgcgctcctccttagcaacctgatgttcttgctcctcaagaacgcaccccagggtaaaatgtccaacggctgacagtgcgtaaagggcattttcaagtgagaacccctgttggcacaagaaagccaactggttctcgagagtctcgtattgtttctctgttggcctggttcccaggtgcacttttgctccatccctatgggacaaaagtgcgcagcggtaggactttgcattgtttcggagaaaatcttgccaactctcgacctcaagggggcaggaatgagtgtgatgcctgtcaagcatttcaataggaagagcgtccagcagggctcttttgttcttgacatgccaatacaatgtaggttgctccacacccaatttctgggcgagctttcgagtagtcagaccttcgatgcccacgccgttgaggagttcgagtgcgctatttattaccttgctcttatccaagcgggacatggtggcgtttaatgcggccgcttcgtctaacaaaaaagccaaaaacggccagaatttagcggacaatttactagtctaacactgaaaattacatattgacccaaatgattacatttcaaaaggtgcctaaaaaacttcacaaaacacactcgccaaccccgagcgcatagttcaaaaccggagcttcagctacttaagaagataggtacataaaaccgaccaaagaaactgacgcctcacttatccctcccctcaccagaggtccggcgcctgtcgattcaggagagcctaccctaggcccgaaccctgcgtcctgcgacggagaaaagcctaccgcacacctaccggcaggtggccccaccctgcattataagccaacagaacgggtgacgtcacgacacgacgaggcgcgcgctcccaaaggtacgggtgcactgcccaacggcaccgcataactgccgcccccgcaacagacgacaaaccgagttctccagtcagtgacaaacttcacgtcagggtccccagatggtgccccagcccatctcacccgaataagagctttcccgcattagcgaaggcctcaagaccttgggttcttgccgcccaccatgccccccaccttgtttcaacgacctcacagcccgcctcacaagcgtcttccattcaagactcgggaacagccgccattttgctgcgctccccccaacccccagttcagggcaaccttgctcgcggacccagactacagcccttggcggtctctccacacgcttccgtcccaccgagcggcccggcggccacgaaagccccggccagcccagcagcccgctactcaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccagatcaggaattccgatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaaatctggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttaccattactacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataa

(19) United States
(12) Patent Application Publication
(10) Pub. No.: US 2026/0218208 A1
(43) Pub. Date: Jul. 30, 2026

(54) MUTANT REVERSE TETRACYCLINE TRANSACTIVATORS FOR EXPRESSION OF GENES

(71) Applicant: President and Fellows of Harvard College, Cambridge, MA (US)

(72) Inventors: David A. SINCLAIR, Cambridge, MA (US); Yuancheng LU, Cambridge, MA (US); Noah Justin DAVIDSOHN, La Jolla, CA (US)

(73) Assignee: President and Fellows of Harvard College, Cambridge, MA (US)

(21) Appl. No.: 19/642,752

(22) Filed: Apr. 9, 2026

Related U.S. Application Data
(62) Division of application No. 17/280,294, filed on Sep. 27, 2019, now Pat. No. 12,624,362.
(60) Provisional application No. 62/738,894, filed on Sep. 28, 2018.

(51) Int. Cl. C12N 15/63; A61K 48/00; C12N 15/86

(52) U.S. Cl. CPC C12N 15/635; A61K 48/0058; C12N 15/86; C12N 2750/14143; C12N 2750/14171; C12N 2830/003

(57) ABSTRACT

Provided herein are mutant reverse tetracycline transactivator (rtTA) proteins and engineered nucleic acids (e.g., viral vectors, including lentiviral vectors, adenoviral vectors, AAV vectors, herpes viral vectors, and retroviral vectors, and non-viral vectors, including RNA and plasmid DNA) that encode a mutant rtTA that are useful, for example, in regulating gene expression, inducing cellular reprogramming, tissue repair, tissue regeneration, organ regeneration, reversing aging, treating a disease (e.g., acute injuries, neurodegenerative disease, chronic diseases, proliferative diseases, cardiovascular diseases, genetic diseases, inflammatory diseases, autoimmunue diseases, neurological diseases, hematological diseases, painful conditions, psychiatric disorders, metabolic disorders, cancers, aging, age-related diseases, and diseases affecting any tissue in a subject), or any combination thereof. Also provided herein are recombinant viruses comprising the engineered nucleic acids and methods of regulating (e.g., inhibiting or inducing) cellular reprogramming, tissue repair, tissue regeneration, or any combination thereof.

### RELATED APPLICATIONS

This application is a divisional under 35 U.S.C. § 121 of co-pending U.S. application Ser. No. 17/280,294 filed Mar. 26, 2021, which is a national stage filing under 35 U.S.C. § 371 of International PCT application number PCT/US2019/053492 filed Sep. 27, 20219, which claims the benefit under 35 U.S.C. § 119(e) of U.S. provisional application No. 62/738,894, filed Sep. 28, 2018, each of which is incorporated by reference herein in its entirety.

### SEQUENCE LISTING

The instant application contains a Sequence Listing which has been submitted electronically in XML file format and is hereby incorporated by reference in its entirety. Said XML copy, created on Apr. 7, 2026, is named 002806-000180USD1_SL.xml and is 105,663 bytes in size.

### BACKGROUND OF THE INVENTION

Inducible gene expression holds great promise for gene therapy and other biomedical applications. On-demand and tightly regulated gene expression can obviate toxicity associated with prolonged expression or toxicity associated with super physiological expression of exogenous genes.

As an example, tetracycline-on (Tet-On) systems often use a reverse tetracycline transactivator (rtTA) to induce gene expression. Reverse tetracycline transactivators (rtTAs) comprise a mutant tetracycline repressor DNA binding protein (TetR) and a transactivation domain (e.g., Gossen et al., Science. 1995 Jun. 23; 268(5218):1766-9). These transactivators can be activated in the presence of a tetracycline (e.g., doxycycline) and subsequently bind to promoters comprising a tetracycline-responsive element (TRE) to induce gene expression (Gossen et al., Science. 1995 Jun. 23; 268(5218):1766-9); Baron et al., Methods Enzymol. 2000; 327:401-21. A TRE comprises at least one Tet operator (Tet-O) sequence (e.g., multiple repeats of Tet-O sequences) and may be located upstream of a minimal promoter (e.g., minimal promoter sequence derived from the human cytomegalovirus (hCMV) immediate-early promoter).

The tendency of Tet-On systems to turn on gene expression (be “leaky”) even in the absence of a tetracycline, however, has hindered their use. Therefore, improved Tet-On systems with little to no leakiness and high tetracycline sensitivity are needed.

### SUMMARY OF THE INVENTION

The present disclosure stems from the unexpected discovery that four mutations in the residues corresponding to positions G72, G12, F67, and R171 in rtTA3 (SEQ ID NO: 11) significantly improve the sensitivity and lower the leakiness of Tet-On systems in vivo. Provided herein, in some embodiments, are mutant rtTAs (e.g., rtTA4), engineered nucleic acids (e.g., expression vectors, including viral and non-viral vectors) encoding the same, recombinant viruses (e.g., lentivirus, adenovirus, alphavirus, vaccinia virus, retrovirus, herpes virus, or AAV) comprising the engineered nucleic acids (e.g., expression vectors), pharmaceutical compositions, and kits thereof. In some embodiments, the engineered nucleic acids encoding a mutant rtTA4 further encodes a transgene (e.g., a protein-encoding sequence, a gene-targeting nucleic acid, and/or a therapeutic sequence) operably linked to a tetracycline-responsive element (TRE) promoter. In some embodiments, the pharmaceutical compositions and kits further comprise a second vector (e.g., multiple second vectors) or a second recombinant virus (e.g., multiple second recombinant viruses) (e.g., lentivirus, adenovirus, alphavirus, vaccinia virus, retrovirus, herpes virus, or AAV) that comprises a tetracycline-responsive element (TRE) promoter operably linked to a transgene. In some embodiments, the pharmaceutical compositions and kits further comprise multiple vectors or multiple recombinant viruses (e.g., lentiviruses, vaccinia viruses, alphaviruses, adenoviruses, retroviruses, herpes viruses, or adeno-associated viruses (AAV)) that comprise a tetracycline-responsive element (TRE) promoter operably linked to a transgene. Methods of promoting gene expression comprising administering (1) any of the engineered nucleic acids (e.g., expression vectors) described herein that encode a mutant rtTA (e.g., rtTA4) and (2) tetracycline to a cell, tissue, or subject in need thereof are also provided herein. In certain embodiments, the methods further comprise administering (3) a second nucleic acid (e.g., an engineered nucleic acid, including an expression vector) comprising a TRE promoter operably linked to a transgene. In certain embodiments, multiple second engineered nucleic acids (e.g., expression vectors) are administered.

The mutant rtTAs (e.g., rtTA4s), engineered nucleic acids (e.g., expression vectors), recombinant viruses, pharmaceutical compositions, kits, and methods described herein are useful in regulating gene expression in vivo. Without being bound by a particular theory, the reduction in leakiness improves the toxicity profile of the rtTA4 Tet-On system compared to rtTA3 Tet-On system and allows for transient expression of transgenes.

Aspects of the present disclosure provide mutant rtTAs (e.g., rtTA4). A mutant rtTA of the present disclosure comprises four mutations corresponding to positions G72, G12, F67, and R171 of rtTA3 (SEQ ID NO: 11), and such a mutant rtTA comprising four mutations at these positions is referred to as rtTA4. In certain embodiments, a mutant rtTA further comprises at least one (e.g., at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 80, 90, 100, 110, 120, 130, 140, 150, 160, 170, 180, 190, 200, 210, 220, 230, or 240) mutation in a residue corresponding to a position in rtTA3 (SEQ ID NO: 11) that is not G72, G12, F67, or R171. In certain embodiments, the mutation in a residue corresponding to a position in rtTA3 (SEQ ID NO: 11) that is not positions G72, G12, F67, or R171 is a point mutation, truncation mutation, deletion, or insertion.

In certain embodiments, the G72 mutation is G72V, G72I, G72L, or G72P; the G12 mutation is G12S or G12T; the F67 mutation is F67S or F67T; and the R171 mutation is R171K or R171H.

In certain embodiments, the four mutations are G72V or G72P, G12S, F67S, and R171K. An amino acid sequence encoding mutant rtTA (e.g., rtTA4) may comprise a sequence that is at least 70% (e.g., at least 75%, 80%, 85%, 90%, 95%, 98%, 99%, or 100%) identical to SEQ ID NO: 13 and comprises a mutation in a residue corresponding to the following positions in rtTA3 (SEQ ID NO: 11): G72, G12, F67, and R171. A nucleic acid encoding mutant rtTA (e.g., rtTA4) may be codon optimized and may comprise a sequence that is at least 70% (e.g., at least 75%, 80%, 85%, 90%, 95%, 98%, 99%, or 100%) identical to SEQ ID NO: 12.

In another aspect of the present disclosure, a nucleic acid (e.g., an engineered nucleic acid, including an expression vector) (e.g., viral expression vector, including lentiviral, retroviral, adenoviral, herpes virus, or adeno-associated virus (AAV)) comprises a nucleic acid sequence encoding a mutant rtTA (e.g., rtTA4) operably linked to a promoter (e.g., constitutive or tissue-specific promoter). In certain embodiments, a nucleic acid (e.g., an engineered nucleic acid, including an expression vector) encoding a mutant rtTA (e.g., rtTA4) comprises a sequence that is at least 70% (e.g., at least 75%, 80%, 85%, 90%, 95%, 98%, 99%, or 100%) identical to SEQ ID NO: 17. In certain embodiments, a nucleic acid (e.g., an engineered nucleic acid, including an expression vector) encoding a mutant rtTA (e.g., rtTA4) consists of SEQ ID NO: 17. In certain embodiments, a nucleic acid (e.g., an engineered nucleic acid, including an expression vector) encoding a mutant rtTA (e.g., rtTA4) comprises a sequence that is at least 70% (e.g., at least 75%, 80%, 85%, 90%, 95%, 98%, 99%, or 100%) identical to a desmin-rtTA4 vector (SEQ ID NO: 30). In certain embodiments, a nucleic acid (e.g., an engineered nucleic acid, including an expression vector) encoding a mutant rtTA (e.g., rtTA4) consists of SEQ ID NO: 30.

The promoter operably linked to the nucleic acid encoding a mutant rtTA (e.g., rtTA4) may be a constitutive promoter (e.g., CP1, CMV, EF1a, SV40, PGK1, Ubc, human beta actin, CAG, Ac5, polyhedrin, TEF1, GDS, CaM3 5S, Ubi, H1, or U6 promoter). To allow for tissue-specific expression of a rtTA, the promoter may be tissue-specific (e.g., an eye-specific promoter, a bone-specific promoter, a lung-specific promoter, a breast-specific promoter, a pancreas-specific promoter, a muscle-specific promoter, a liver-specific promoter, a skin-specific promoter, a heart-specific promoter, a brain-specific promoter, a nerve tissue-specific promoter, a kidney-specific promoter, a testes-specific promoter, an ovary-specific promoter, or an intestine-specific promoter).

In certain embodiments, the nucleic acid (e.g., an engineered nucleic acid, including an expression vector) encoding a mutant rtTA (e.g., rtTA4) further encodes a tetracycline repressor (e.g., tetR, tetRKRAB, TRSID), which can prevent rtTA binding to a TRE promoter in the absence of a tetracycline. In certain embodiments, the nucleic acid sequence encoding a mutant rtTA (e.g., rtTA4) and the nucleic acid sequence encoding a tetracycline repressor (e.g., tetRKRAB) is operably linked to the same promoter. In certain embodiments, a separator sequence (e.g., an internal ribosome entry site (IRES) or 2A peptide) is present in a nucleic acid (e.g., an engineered nucleic acid, including an expression vector) and separates at least two nucleic acid sequences, which may be helpful in producing two separate amino acid sequences from one expression vector.

In certain embodiments, a nucleic acid (e.g., an engineered nucleic acid, including an expression vector) encoding mutant rtTA (e.g., rtTA4) further comprises a Woodchuck Hepatitis Virus (WHP) Posttranscriptional Regulatory Element (WPRE), which may be useful in enhancing transgene expression (e.g., from a viral vector). In certain embodiments, a WPRE sequence is at least 70% (e.g., at least 75%, at least 80%, at least 85%, at least 90%, at least 95%, at least 98%, at least 99%, or at least 100%) identical to SEQ ID NO: 21.

In certain embodiments, the nucleic acid (e.g., an engineered nucleic acid, including an expression vector) encoding a mutant rtTA (e.g., rtTA4) is a viral vector (e.g., lentivirus vector, adenovirus vector, vaccinia viruses, alphviruses, adeno-associated virus vector, adeno-associated virus (AAV) vector, or retrovirus vector). In certain embodiments, an AAV vector is an AAV1, AAV2, AAV3, AAV4, AAV5, AAV6, AAV7, AAV8, AAV9, or AAV10 vector. A viral vector (e.g., AAV vector) may further comprise inverted terminal repeat sequences (ITRs). In certain embodiments, an ITR comprises a sequence that is at least 70% identical to (e.g., at least 75%, at least 80%, at least 85%, at least 90%, at least 95%, at least 98%, at least 99%, or at least 100%) identical to SEQ ID NO: 22.

Another aspect of the present disclosure provides recombinant viruses harboring any engineered nucleic acids (e.g., expression vectors) encoding mutant rtTA (e.g., rtTA4).

In yet another aspect of the present disclosure, pharmaceutical compositions comprising any of the mutant rtTAs (e.g., rtTA4) and a pharmaceutically acceptable excipient are provided. The pharmaceutical composition may further comprise a second nucleic acid (e.g., an engineered nucleic acid, including an expression vector) encoding a transgene. The second nucleic acid (e.g., an engineered nucleic acid, including an expression vector) comprises a tetracycline-responsive element (TRE) promoter (e.g., a TRE3G sequence that is at least 70%, least 75%, at least 80%, at least 85%, at least 90%, at least 95%, at least 98%, at least 99%, or at least 100% identical to SEQ ID NO: 7) operably linked to the transgene. In some embodiments, the TRE promoter is a TRE2 or P tight promoter. In some embodiments, a TRE promoter is a TRE2 promoter and comprises a sequence that is at least 70%, least 75%, at least 80%, at least 85%, at least 90%, at least 95%, at least 98%, at least 99%, or at least 100% identical to SEQ ID NO: 23. In some embodiments, a TRE promoter is a P tight promoter and comprises a sequence that is at least 70%, least 75%, at least 80%, at least 85%, at least 90%, at least 95%, at least 98%, at least 99%, or at least 100% identical to SEQ ID NO: 24.

In certain embodiments, a transgene is any protein-encoding gene. In certain embodiments, a transgene is a gene-targeting nucleic acid. In certain embodiments, a transgene is a therapeutic sequence. In certain embodiments, a therapeutic sequence may be useful in treating acute injuries, neurodegenerative disease, chronic diseases, proliferative diseases, cardiovascular diseases, genetic diseases, inflammatory diseases, autoimmunue diseases, neurological diseases, hematological diseases, painful conditions, psychiatric disorders, metabolic disorders, cancers, aging, age-related diseases, and diseases affecting any tissue in a subject.

In certain embodiments, the inducible nucleic acid (e.g., an engineered nucleic acid, including an expression vector) (e.g., viral vector) encodes OCT4, SOX2, and KLF4. See, e.g., the U.S. Provisional application entitled CELLULAR REPROGRAMMING TO REVERSE AGING AND PROMOTE ORGAN AND TISSUE REGENERATION under attorney docket number H0824.70296US00, U.S. Ser. No. 62/738,922, which was filed on Sep. 28, 2018, U.S. Ser. No. 62/792,283, entitled CELLULAR REPROGRAMMING TO REVERSE AGING AND PROMOTE ORGAN AND TISSUE REGENERATION, filed Jan. 14, 2019, U.S. Ser. No. 62/865,877, entitled CELLULAR REPROGRAMMING TO REVERSE AGING AND PROMOTE ORGAN AND TISSUE REGENERATION filed Jun. 24, 2019, and U.S. Ser. No. 62/880,488, entitled CELLULAR REPROGRAMMING TO REVERSE AGING AND PROMOTE ORGAN AND TISSUE REGENERATION, filed Jul. 30, 2019, and the PCT application entitled CELLULAR REPROGRAMMING TO REVERSE AGING AND PROMOTE ORGAN AND TISSUE REGENERATION under attorney docket number H0824.70296WO00, which was filed on the same day as the instant application, each of which is incorporated by reference herein in its entirety

In certain embodiments, the nucleic acid (e.g., an engineered nucleic acid, including an expression vector) encoding a mutant rtTA (e.g., rtTA4) and the nucleic acid (e.g., an engineered nucleic acid, including an expression vector) encoding a transgene operably linked to a TRE promoter are both viral vectors and are in viruses (e.g., lentivirus, adenovirus, alphavirus, vaccinia virus, retrovirus, herpes virus, or AAV). In certain embodiments, a pharmaceutical composition comprises both viral vectors and/or both viruses.

In yet another aspect of the present disclosure, cells and/or systems are provided comprising any of the mutant rtTAs (e.g., rtTA4), any of the engineered nucleic acids (e.g., expression vectors) encoding a mutant rtTA and/or a transgene operably linked to a TRE promoter, and/or any of the viruses (e.g., lentivirus, adenovirus, alphavirus, vaccinia virus, retrovirus, herpes virus, or AAV).

In yet another aspect of the present disclosure, kits are provided comprising any of the mutant rtTAs (e.g., rtTA4), any of the engineered nucleic acids (e.g., expression vectors) encoding a mutant rtTA, any of the viruses (e.g., lentivirus, adenovirus, alphavirus, vaccinia virus, retrovirus, herpes virus, or AAV), cells, systems, and/or any of the pharmaceutical compositions described herein.

Another aspect of the present disclosure provides methods for promoting gene expression comprising administering (1) any of the mutant rtTAs (e.g., rtTA4), any of the engineered nucleic acids (e.g., expression vectors) encoding a mutant rtTA (e.g., rtTA4), or a recombinant virus comprising any of the engineered nucleic acids (e.g., expression vectors) encoding a mutant rtTA; (2) any of the engineered nucleic acids (e.g., expression vectors) encoding a transgene operably linked to a TRE promoter; and (3) a tetracycline (e.g., doxycycline) to a cell, tissue, or a subject in need thereof. In certain embodiments, the engineered nucleic acids (e.g., expression vectors) encoding a mutant rtTA or a recombinant virus comprising any of the mutant rtTA expression vectors described herein further encodes a tetracycline repressor (e.g., tetRKRAB). In certain embodiments, a nucleic acid (e.g., an engineered nucleic acid, including an expression vector) encoding a mutant rtTA or a recombinant virus of the present disclosure further comprises a transgene operably linked to a TRE promoter (e.g., TRE3G, a TRE2, or a P tight promoter). The method may further comprise withdrawing (i.e., ceasing the administration of) the tetracycline.

In certain embodiments, the subject is a mammal (e.g., human or non-human). In certain embodiments, the subject has a disease (e.g., an acute injury, a neurodegenerative disease, a chronic disease, a proliferative disease, a cardiovascular disease, a genetic disease, an inflammatory disease, an autoimmunue disease, a neurological disease, a hematological disease, a painful condition, a psychiatric disorder, a metabolic disorder, cancer, aging, an age-related disease, and any disease affecting any tissue in a subject). In certain embodiments, the method comprises regulating cellular reprogramming, tissue repair, treating a disease (e.g., an acute injury, a neurodegenerative disease, a chronic disease, a proliferative disease, a cardiovascular disease, a genetic disease, an inflammatory disease, an autoimmunue disease, a neurological disease, a hematological disease, a painful condition, a psychiatric disorder, a metabolic disorder, cancer, aging, an age-related disease, and any disease affecting any tissue in a subject), tissue regeneration, organ regeneration, reversing aging, or any combination thereof.

The details of one or more embodiments of the invention are set forth herein. Other features, objects, and advantages of the invention will be apparent from the Detailed Description, Examples, Figures, and Claims.

References cited in this application are incorporated herein by reference.

### Definitions

“AAV” or “adeno-associated virus” is a nonenveloped virus that is capable of carrying and delivering nucleic acids (e.g., nucleic acids encoding a transgene, a mutant rtTA4, or any combination thereof) and belongs to the genus Dependoparvovirus. In general, AAV does not integrate into the genome. The tissue-specific targeting capabilities of AAV is often determined by the AAV capsid serotype (see, e.g., Table 1 below for examples of AAV serotypes and their utility in tissue-specific delivery). Non-limiting serotypes of AAV include AAV1, AAV2, AAV3, AAV4, AAV5, AAV6, AAV7, AAV8, AAV9, AAV10, AAV11, and variants thereof. In certain embodiments, the AAV serotype is a variant of AAV9 (e.g., AAV PHP.b).

A “recombinant virus” is a virus (e.g., lentivirus, adenovirus, alphavirus, vaccinia virus, retrovirus, herpes virus, or adeno-associated (AAV)) that has been isolated from its natural environment (e.g., from a host cell, tissue, or a subject) or is artificially produced.

The term “AAV vector,” as used herein, is a nucleic acid that comprises AAV inverted terminal repeats (ITRs) flanking an expression cassette (e.g., an expression cassette comprising a nucleic acid encoding a transgene alone or in combination or an expression cassette encoding rtTA or tTA). An AAV vector may further comprise a promoter sequence.

The terms “administer,” “administering,” or “administration,” as used herein refers to introduction of a nucleic acid (e.g., an engineered nucleic acid encoding a transgene and/or encoding a mutant rtTA), a recombinant cell, a recombinant virus (e.g., lentivirus, adenovirus, alphavirus, vaccinia virus, retrovirus, herpes virus, or AAV), a mutant rtTA (e.g., rtTA4), or any combination thereof or a pharmaceutical composition thereof. An engineered nucleic acid, recombinant cell, mutant rtTA4, virus (e.g., lentivirus, adenovirus, alphavirus, vaccinia virus, retrovirus, herpes virus, or AAV), or pharmaceutical compositions thereof may be administered intravenously, intradermally, intraarterially, intralesionally, intratumorally, intracranially, intraarticularly, intraprostaticaly, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, systemically, injection, infusion, continuous infusion, localized perfusion bathing target cells directly, via a catheter, in creams, in lipid compositions (e.g., liposomes), or by other method or any combination of the forgoing as would be known to one of ordinary skill in the art (see, for example, Remington's Pharmaceutical Sciences (1990), incorporated herein by reference). The nucleic acids (e.g., engineered nucleic acids, including expression vectors), recombinant cells, mutant rtTA protein, or recombinant viruses (e.g., lentivirus, adenovirus, alphavirus, vaccinia virus, retrovirus, herpes virus, or AAV) of the present disclosure may be administered to any tissue, cell, organ, or subject.

The term “epigenome” or “epigenetics” refers to the modification and structural changes within a cell that control the expression of nucleic acids (e.g., engineered nucleic acids) or genomic information in a cell. Changes to the epigenome occur during, and drive the processes of embryonic development, disease progression, and aging.

The term “cellular senescence” refers to a cell that has exited the cell cycle, displays epigenetic markers consistent with senescence, or expressing senescence cell markers (e.g. senescence-associated beta-galactosidase, or inflammatory cytokines). Cellular senescence may be partial or complete.

The term “gene expression” refers to the degree to which certain genes or all genes in a cell or tissue are transcribed into RNA. In some instances, the RNA is translated by the cell into a protein. The epigenome dictates gene expression patterns.

The term “cellular reprogramming” refers to the process of altering the epigenome of a cell using reprogramming factors (e.g. reversing or preventing epigenetic changes in cells that are causes of dysfunction, deterioration, cell death, senescence or aging). Cellular reprogramming may be complete reprogramming, such that a differentiated cell (e.g., somatic cell) is reprogrammed to a pluripotent stem cell. Cellular reprogramming may be incomplete, such that a differentiated cell (e.g., somatic cell) retains its cellular identity (e.g., lineage-specific stem cell). Cellular reprogramming may be incomplete, e.g., a stem cell is not created,] such that a cell is rejuvenated, or takes on more youthful attributes (e.g. increased survival, reduced inflammation, or ability to divide). Cellular reprogramming may provide additional cellular functions, or prevent cellular aging (e.g., transdifferentiation, or transition into cellular senescence). Cellular reprogramming may induce temporary or permanent gene expression changes. In some embodiments, incomplete cellular reprogramming is shown by the lack of Nanog expression. In some embodiments, cellular reprogramming prevents senescence from occurring.

The terms “condition,” “disease,” and “disorder” are used interchangeably. Non-limiting examples of conditions, diseases, and disorders include acute injuries, neurodegenerative disease, chronic diseases, proliferative diseases, cardiovascular diseases, genetic diseases, inflammatory diseases, autoimmunue diseases, neurological diseases, hematological diseases, painful conditions, psychiatric disorders, metabolic disorders, cancers, aging, age-related diseases, and diseases affecting any tissue in a subject. For example, age-related conditions include, heart failure, stroke, heart disease, atherosclerosis, neurodegenerative diseases (e.g., Parkinson's disease and Alzheimer's disease), cognitive decline, memory loss, diabetes, osteoporosis, arthritis, muscle loss, hearing loss (partial or total), eye-related conditions (e.g., poor eye sight or retinal disease), glaucoma, and cancer. In certain embodiments, the disease is a retinal disease (e.g., macular degeneration). In some embodiments, an age-related condition is senescence. As a non-limiting example, senescence of glial cells may be a cause of Alzheimer's disease. See e.g., Bussian, et al., Nature. 2018 Sep. 19.

As used herein, an “ocular disease” or “eye disease” is a disease or condition of the eye. Non-limiting examples of conditions that affect the eye include Ectropion, Lagophthalmos, Blepharochalasis, Ptosis, Stye, Xanthelasma, Dermatitis, Demodex, leishmaniasis, loiasis, onchocerciasis, phthiriasis, (herpes simplex), leprosy, molluscum contagiosum, tuberculosis, yaws, zoster, impetigo, Dacryoadenitis, Epiphora, exophthalmos, Conjunctivitis, Scleritis, Keratitis, Corneal ulcer/Corneal abrasion, Snow blindness/Arc eye, Thygeson's superficial punctate keratopathy, Corneal neovascularization, Fuchs' dystrophy, Keratoconus, Keratoconjunctivitis sicca, Iritis, iris, Uveitis, Sympathetic ophthalmia, Cataract, lens, Chorioretinal inflammation, Focal chorioretinal inflammation, chorioretinitis, choroiditis, retinitis, retinochoroiditis, Disseminated chorioretinal inflammation, exudative retinopathy, Posterior cyclitis, Pars planitis, chorioretinal inflammations, Harada's disease, Chorioretinal inflammation, choroid, Chorioretinal scars, Macula scars, posterior pole (postinflammatory) (post-traumatic), Solar retinopathy, Choroidal degeneration, Atrophy, Sclerosis, angioid streaks, choroidal dystrophy, Choroideremia, choroidal, areolar, (peripapillary), Gyrate atrophy, choroid, ornithinaemia, Choroidal haemorrhage, Choroidal haemorrhage, NOS (Not Otherwise Specified), Choroidal detachment, Chorioretinal, Chorioretinal inflammation, infectious and parasitic diseases, Chorioretinitis, syphilitic, toxoplasma, tuberculosis, chorioretinal, Retinal detachment, retina, choroid, distorted vision, Retinoschisis, Hypertensive retinopathy, Diabetic retinopathy, Retinopathy, Retinopathy of prematurity, Age-related macular degeneration, macula, Macular degeneration, Bull's Eye Maculopathy, Epiretinal membrane, Peripheral retinal degeneration, Hereditary retinal dystrophy, Retinitis pigmentosa, Retinal haemorrhage, retinal layers, Central serous retinopathy, Retinal detachment, retinal disorders, Macular edema, macula, Retinal disorder, Diabetic retinopathy, Glaucoma, optic neuropathy, ocular hypertension, open-angle glaucoma, angle-closure glaucoma, Normal Tension glaucoma, open-angle glaucoma, angle-closure glaucoma, Floaters, Leber's hereditary optic neuropathy, Optic disc drusen, Strabismus, Ophthalmoparesis, eye muscles, Progressive external ophthaloplegia, Esotropia, Exotropia, Disorders of refraction, accommodation, Hypermetropia, Myopia, Astigmatism, Anisometropia, Presbyopia, ophthalmoplegia, Amblyopia, Leber's congenital amaurosis, Scotoma, Anopsia, Color blindness, Achromatopsia/Maskun, cone cells, Nyctalopia, Blindness, River blindness, Micropthalmia/coloboma, optic nerve, brain, spinal cord, Red eye, Argyll Robertson pupil, pupils, Keratomycosis, Xerophthalmia, and Aniridia. In some embodiments, the ocular disease is acute or chronic eye injury.

In some embodiments, the ocular disease is a scratched cornea.

In some embodiments, an ocular disease is a corneal disease (e.g., a disease affecting the cornea or corneal cells). In some embodiments, an ocular disease is acanthamoeba keratitis, ectropion, lagoph amblyopia, anisocoria, astigmatism, Bell's Palsy, blepharitis, blurry vision, burning eyes, cataracts, macular degeneration, age-related macular degeneration, diabetic eye disease, glaucoma, dry eye, poor vision (e.g., low vision), astigmatism, blepharitis, cataract, chalazion, conjunctivitis, diabetic retinopathy, dry eye, glaucoma, keratitis, keratonconus, macular degeneration, ocular hypertension, pinquecula, pterygium, retinitis pigmentosa, or ocular cancer (e.g., retinoblastoma, melanoma of the eye, lymphoma of the eye, medulloepithelioma, squamous cell cancer of the conjunctiva). Examples of corneal diseases include, but are not limited to, corneal neovascularization (NV), corneal dystrophy, corneal inflammation, corneal abrasion, and corneal fibrosis. In some embodiments, the ocular disease is Keritaconus. In some embodiments, an ocular disease is macular degeneration. Additional non-limiting examples of eye diseases may be found in the International Statistical Classification of Diseases and Related Health Problems (e.g., VII Diseases of the eye and adnexa).

An ocular disease may affect any part of the eye and/or adnexa. In some embodiments, the ocular disease is a disorder of the eyelid, lacrimal system and/or orbit. In some embodiments, the ocular disease is a disorders of conjunctiva. In some embodiments, the ocular disease is a disorder of sclera, cornea, iris and/or ciliary body. In some embodiments, the ocular disease is a disorder of the lens. In some embodiments, the ocular disease is a disorder of choroid and/or retina. In some embodiments, the ocular disease is glaucoma. In some embodiments, the ocular disease is a disorder of vitreous body and/or globe. In some embodiments, the ocular disease is a disorder of optic nerve and/or visual pathways. In some embodiments, the ocular disease is a disorder of ocular muscles, binocular movement, accommodation and/or refraction. In some embodiments, the ocular disease is ocular muscles, binocular movement, accommodation and refraction. In some embodiments, the ocular disease is a visual disturbance and/or blindness.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include, but are not limited to, Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

The term “musculoskeletal disease” or “MSD” refers to an injury and/or pain in a subject's joints, ligaments, muscles, nerves, tendons, and structures that support limbs, neck, and back. In certain embodiments, an MSD is a degenerative disease. In certain embodiments, an MSD includes an inflammatory condition. Body parts of a subject that may be associated with MSDs include upper and lower back, neck, shoulders, and extremities (arms, legs, feet, and hands). In certain embodiments, an MSD is a bone disease, such as achondroplasia, acromegaly, bone callus, bone demineralization, bone fracture, bone marrow disease, bone marrow neoplasm, dyskeratosis congenita, leukemia (e.g., hairy cell leukemia, lymphocytic leukemia, myeloid leukemia, Philadelphia chromosome-positive leukemia, plasma cell leukemia, stem cell leukemia), systemic mastocytosis, myelodysplastic syndromes, paroxysmal nocturnal hemoglobinuria, myeloid sarcoma, myeloproliferative disorders, multiple myeloma, polycythemia vera, pearson marrow-pancreas syndrome, bone neoplasm, bone marrow neoplasm, Ewing sarcoma, osteochondroma, osteoclastoma, osteosarcoma, brachydactyly, Camurati-Engelmann syndrome, Craniosynostosis, Crouzon craniofacial dysostosis, dwarfism, achondroplasia, bloom syndrome, Cockayne syndrome, Ellis-van Creveld syndrome, Seckel syndrome, spondyloepiphyseal dysplasia, spondyloepiphyseal dysplasia congenita, Werner syndrome, hyperostosis, osteophyte, Klippel-Trenaunay-Weber syndrome, Marfan syndrome, McCune-Albright syndrome, osteitis, osteoarthritis, osteochondritis, osteochondrodysplasia, Kashin-Beck disease, Leri-Weill dyschondrosteosis, osteochondrosis, osteodystrophy, osteogenesis imperfecta, osteolysis, Gorham-Stout syndrome, osteomalacia, osteomyelitis, osteonecrosis, osteopenia, osteopetrosis, osteoporosis, osteosclerosis, otospondylomegaepiphyseal dysplasia, pachydermoperiostosis, Paget disease of bone, Polydactyly, Meckel syndrome, rickets, Rothmund-Thomson syndrome, Sotos syndrome, spondyloepiphyseal dysplasia, spondyloepiphyseal dysplasia congenita, syndactyly, Apert syndrome, syndactyly type II, or Werner syndrome. In certain embodiments, an MSD is a cartilage disease, such as cartilage neoplasm, osteochondritis, osteochondrodysplasia, Kashin-Beck disease, or Leri-Weill dyschondrosteosis. In certain embodiments, an MSD is hernia, such as intervertebral disk hernia. In certain embodiments, an MSD is a joint disease, such as arthralgia, arthritis (e.g., gout (e.g., Kelley-Seegmiller syndrome, Lesch-Nyhan syndrome), Lyme disease, osteoarthritis, psoriatic arthritis, reactive arthritis, rheumatic fever, rheumatoid arthritis, Felty syndrome, synovitis, Blau syndrome, nail-patella syndrome, spondyloarthropathy, reactive arthritis, Stickler syndrome, synovial membrane disease, synovitis, or Blau syndrome. In certain embodiments, an MSD is Langer-Giedion syndrome. In certain embodiments, an MSD is a muscle disease, such as Barth syndrome, mitochondrial encephalomyopathy, MELAS syndrome, MERRF syndrome, MNGIE syndrome, mitochondrial myopathy, Kearns-Sayre syndrome, myalgia, fibromyalgia, polymyalgia rheumatica, myoma, myositis, dermatomyositis, neuromuscular disease, Kearns-Sayre syndrome, muscular dystrophy, myasthenia, congenital myasthenic syndrome, Lambert-Eaton myasthenic syndrome, myasthenia gravis, myotonia, myotonia congenita, spinal muscular atrophy, tetany, ophthalmoplegia, or rhabdomyolysis. In certain embodiments, an MSD is Proteus syndrome. In certain embodiments, an MSD is a rheumatic diseases, such as arthritis (e.g., gout (e.g., Kelley-Seegmiller syndrome, Lesch-Nyhan lyme disease)), osteoarthritis, psoriatic arthritis, reactive arthritis, rheumatic fever, rheumatoid arthritis, Felty syndrome, synovitis, Blau syndrome, gout (e.g., Kelley-Seegmiller syndrome, Lesch-Nyhan syndrome), polymyalgia rheumatica, rheumatic fever, rheumatic heart disease, or Sjogren syndrome. In certain embodiments, an MSD is Schwartz-Jampel syndrome. In certain embodiments, an MSD is a skeleton disease, such as Leri-Weill dyschondrosteosis, skeleton malformations, Melnick-Needles syndrome, pachydermoperiostosis, Rieger syndrome, spinal column disease, intervertebral disk hernia, scoliosis, spina bifida, spondylitis, ankylosing spondylitis, spondyloarthropathy, reactive arthritis, spondyloepiphyseal dysplasia, spondyloepiphyseal dysplasia congenita, or spondylosis. In some embodiments, the disease is a musculoskeletal disease.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation. In some embodiments, the inflammatory disease is inflammaging (e.g., inflammation that is a side effect of aging).

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barre syndrome, Hashimoto's thyroiditis, and cardiomyopathy.

The term “liver disease” or “hepatic disease” refers to damage to or a disease of the liver. Non-limiting examples of liver disease include intrahepatic cholestasis (e.g., alagille syndrome, biliary liver cirrhosis), fatty liver (e.g., alcoholic fatty liver, Reye's syndrome), hepatic vein thrombosis, hepatolenticular degeneration (i.e., Wilson's disease), hepatomegaly, liver abscess (e.g., amebic liver abscess), liver cirrhosis (e.g., alcoholic, biliary, and experimental liver cirrhosis), alcoholic liver diseases (e.g., fatty liver, hepatitis, cirrhosis), parasitic liver disease (e.g., hepatic echinococcosis, fascioliasis, amebic liver abscess), jaundice (e.g., hemolytic, hepatocellular, cholestatic jaundice), cholestasis, portal hypertension, liver enlargement, ascites, hepatitis (e.g., alcoholic hepatitis, animal hepatitis, chronic hepatitis (e.g., autoimmune, hepatitis B, hepatitis C, hepatitis D, drug induced chronic hepatitis), toxic hepatitis, viral human hepatitis (e.g., hepatitis A, hepatitis B, hepatitis C, hepatitis D, hepatitis E), granulomatous hepatitis, secondary biliary cirrhosis, hepatic encephalopathy, varices, primary biliary cirrhosis, primary sclerosing cholangitis, hepatocellular adenoma, hemangiomas, bile stones, liver failure (e.g., hepatic encephalopathy, acute liver failure), angiomyolipoma, calcified liver metastases, cystic liver metastases, fibrolamellar hepatocarcinoma, hepatic adenoma, hepatoma, hepatic cysts (e.g., Simple cysts, Polycystic liver disease, hepatobiliary cystadenoma, choledochal cyst), mesenchymal tumors (mesenchymal hamartoma, infantile hemangioendothelioma, hemangioma, peliosis hepatis, lipomas, inflammatory pseudotumor), epithelial tumors (e.g., bile duct hamartoma, bile duct adenoma), focal nodular hyperplasia, nodular regenerative hyperplasia, hepatoblastoma, hepatocellular carcinoma, cholangiocarcinoma, cystadenocarcinoma, tumors of blood vessels, angiosarcoma, Karposi's sarcoma, hemangioendothelioma, embryonal sarcoma, fibrosarcoma, leiomyosarcoma, rhabdomyosarcoma, carcinosarcoma, teratoma, carcinoid, squamous carcinoma, primary lymphoma, peliosis hepatis, erythrohepatic porphyria, hepatic porphyria (e.g., acute intermittent porphyria, porphyria cutanea tarda), and Zellweger syndrome.

The term “spleen disease” refers to a disease of the spleen. Example of spleen diseases include, but are not limited to, splenomegaly, spleen cancer, asplenia, spleen trauma, idiopathic purpura, Felty's syndrome, Hodgkin's disease, and immune-mediated destruction of the spleen.

The term “lung disease” or “pulmonary disease” refers to a disease of the lung. Examples of lung diseases include, but are not limited to, bronchiectasis, bronchitis, bronchopulmonary dysplasia, interstitial lung disease, occupational lung disease, emphysema, cystic fibrosis, acute respiratory distress syndrome (ARDS), severe acute respiratory syndrome (SARS), asthma (e.g., intermittent asthma, mild persistent asthma, moderate persistent asthma, severe persistent asthma), chronic bronchitis, chronic obstructive pulmonary disease (COPD), emphysema, interstitial lung disease, sarcoidosis, asbestosis, aspergilloma, aspergillosis, pneumonia (e.g., lobar pneumonia, multilobar pneumonia, bronchial pneumonia, interstitial pneumonia), pulmonary fibrosis, pulmonary tuberculosis, rheumatoid lung disease, pulmonary embolism, and lung cancer (e.g., non-small-cell lung carcinoma (e.g., adenocarcinoma, squamous-cell lung carcinoma, large-cell lung carcinoma), small-cell lung carcinoma).

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; bbrain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

A “painful condition” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D.C. (1994). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, Cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

In some embodiments, a disease is characterized by cellular dysfunction. For example, a disease may be a mitochondrial disease. Non-limiting mitochondrial diseases include Freidrich's ataxia, alphers disease, barth syndrome, beta-oxidation defects, carnitine deficiency, CPT I deficiency, and mitochondrial DNA depletion. Cellular dysfunction may include mitochondria dysfunction, RNA replication dysfunction, DNA replication dysfunction, translation dysfunction, and/or protein folding dysfunction.

In some embodiments, the disease or condition by a wood, bleeding out, injuries (e.g., broken bones, gunshot wound, cut, scarring during surgery (e.g., cesarean).

In some embodiments, the disease is an infectious disease (e.g., a disease caused by a pathogen and/or virus). Non-limiting examples of infectious diseases include tuberculosis, HIV/AIDS, rabies, plague, cholera, dengue fever, measles, malaria, meningitis, whooping cough, lyme disease, influenza, hepatitis C, typhoid fever, and poliomyelitis.

The terms “effective amount” and “therapeutically effective amount,” as used herein, refer to the amount or concentration of an inventive compound, that, when administered to a subject, is effective to at least partially treat a condition from which the subject is suffering.

As used herein, a protein that is “functional” or “active” is one that retains its biological activity (e.g., capable of acting as a transcription factor or as an inducing agent). Conversely, a protein that is not functional or is inactive is one that is not capable of performing one or more of its wild-type functions.

A “euakaryotic cell” is a cell that comprises a nucleus that is enclosed by a membrane. Non-limiting examples of eukaryotic cells include animal cells, plant cells fungi or protoctista cells, optionally wherein the animal cell is a mammalian cell.

The term “gene” refers to a nucleic acid fragment that expresses a protein, including regulatory sequences preceding (5′non-coding sequences) and following (3′non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

“Homolog” or “homologous” refers to sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity). Homologous sequences include, but are not limited to, paralogous or orthologous sequences. Paralogous sequences arise from duplication of a gene within a genome of a species, while orthologous sequences diverge after a speciation event. A functional homolog retains one or more biological activities of a wild-type protein. In certain embodiments, a functional homolog of protein encoded by a transgene retains at least 10%, at least 20%, at least 30%, at least 40%, at least 50%, at least 60%, at least 70%, at least 80%, at least 90%, or at least 100% of the biological activity (e.g., transcription factor activity) of a wild-type counterpart.

“Inverted terminal repeats” or “ITRs” are nucleic acid sequences that are reverse complements of one another. In general, in an AAV vector, ITRs are found on either side of a cassette (e.g., an expression cassette comprising a nucleic acid encoding a transgene, a mutant rtTA, or any combination thereof). AAV ITRs include ITRs from AAV1, AAV2, AAV3, AAV4, AAV5, AAV6, AAV7, AAV8, AAV9, AAV10, AAV11, and AAV variants thereof.

The terms “nucleic acid,” “polynucleotide”, “nucleotide sequence”, “nucleic acid molecule”, “nucleic acid sequence”, and “oligonucleotide” refer to a series of nucleotide bases (also called “nucleotides”) in DNA and RNA, and mean any chain of two or more nucleotides. The terms “nucleic acid” or “nucleic acid sequence”, “nucleic acid molecule”, “nucleic acid fragment” or “polynucleotide” may be used interchangeably with “gene”, “mRNA encoded by a gene” and “cDNA”.

The nucleic acids can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

The nucleic acids described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The nucleic acids may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

A “recombinant nucleic acid molecule” or “engineered nucleic acid” is a nucleic acid molecule that has undergone a molecular biological manipulation, i.e., non-naturally occurring nucleic acid molecule or genetically engineered nucleic acid molecule. Furthermore, the terms “recombinant DNA molecule” or “engineered nucleic acid” refer to a nucleic acid sequence which is not naturally occurring, or can be made by the artificial combination of two otherwise separated segments of nucleic acid sequence, i.e., by ligating together pieces of DNA that are not normally continuous. By “recombinantly produced” is meant artificial combination often accomplished by either chemical synthesis means, or by the artificial manipulation of isolated segments of nucleic acids, e.g., by genetic engineering techniques using restriction enzymes, ligases, and similar recombinant techniques as described by, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed. D. N. Glover) IREL Press, Oxford, (1985); each of which is incorporated herein by reference.

Such manipulation may be done to replace a codon with a redundant codon encoding the same or a conservative amino acid, while typically introducing or removing a sequence recognition site. Alternatively, it may be performed to join together nucleic acid segments of desired functions to generate a single genetic entity comprising a desired combination of functions not found in nature. Restriction enzyme recognition sites are often the target of such artificial manipulations, but other site specific targets, e.g., promoters, DNA replication sites, regulation sequences, control sequences, open reading frames, or other useful features may be incorporated by design.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

A “recombinant cell” or an “engineered cell” is a cell comprising a recombinant nucleic acid.

“A residue in sequence X corresponding to position a in sequence Y” refers to the residue at the counterpart position of a in sequence X when sequences X and Y are aligned using an amino acid sequence alignment tools known in the art, for example, Clustal Omega or BLAST®.

The terms “leaky” or “leakiness” when used in reference to an inducible system (e.g., Tet-On system or Tet-Off) refers to expression of a transgene from an inducible promoter in the absence of gene induction. For example, in a Tet-On system, expression of a transgene in the absence of tetracycline (e.g., doxycycline) is considered a “leaky” system. As another example, in a Tet-Off system, expression of a transgene in the presence of tetracycline (e.g., doxycycline) is considered a “leaky” system. The level of “leakiness” of an inducible system may be determined by measuring the level of gene expression (e.g., by western blot, RNA analysis or ELISAs) in the absence of gene induction (e.g., in the absence of a tetracycline in a Tet-On system or in the presence of a tetracycline in a Tet-Off system).

The term “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific, or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation of that sequence, expression of that sequence, or a combination thereof.

A promoter may promote ubiquitous expression or tissue-specific expression of an operably linked nucleic acid sequence from any species, including humans. In some embodiments, the promoter is a eukaryotic promoter. Non-limiting examples of eukaryotic promoters include TDH3, PGK1, PKC1, TDH2, PYK1, TPI1, AT1, CMV, EF1a, SV40, PGK1 (human or mouse), Ubc, human beta actin, CAG, TRE, UAS, Ac5, Polyhedrin, CaMKIIa, GAL1, GAL10, TEF1, GDS, ADH1, CaMV35S, Ubi, H1, and U6, as would be known to one of ordinary skill in the art (see, e.g., Addgene website: blog.addgene.org/plasmids-101-the-promoter-region).

Non-limiting examples of ubiquitous promoters include tetracycline-responsive promoters (under the relevant conditions), CMV, EF1 alpha, a SV40 promoter, PGK1, Ubc, CAG, human beta actin gene promoter, and a promoter comprising an upstream activating sequence (UAS). In certain embodiments, the promoter is a mammalian promoter. Non-limiting examples of tissue-specific promoters include brain-specific, liver-specific, muscle-specific, nerve cell-specific, lung-specific, heart-specific, bone-specific, intestine-specific, skin-specific promoters, brain-specific promoters, and eye-specific promoters. As an example, a muscle-specific promoter is a desmin promoter (e.g., a sequence that is at least 70% (e.g., at least 75%, 80%, 85%, 90%, 95%, 98%, 99%, or 100%) identical to SEQ ID NO: 29).

Non-limiting examples of constitutive promoters include CP1, CMV, EF1 alpha, SV40, PGK1, Ubc, human beta actin, beta tubulin, CAG, Ac5, polyhedrin, TEF1, GDS, CaM3 5S, Ubi, H1, and U6. An Ubc promoter may comprise a sequence at is at least 70% (e.g., at least 75%, 80%, 85%, 90%, 95%, 98%, 99%, or 100%) identical to SEQ ID NO: 18.

An “inducible promoter” is one that is characterized by initiating or enhancing transcriptional activity when in the presence of, influenced by, or contacted by an inducing agent. An inducing agent may be endogenous or a normally exogenous condition, compound, agent, or protein that contacts an engineered nucleic acid in such a way as to be active in inducing transcriptional activity from the inducible promoter. In certain embodiments, an inducing agent is a tetracycline-sensitive protein (e.g., rtTA).

Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (tetRTetR, e.g., SEQ ID NO: 26 or TetRKRAB, e.g., SEQ ID NO: 27), a tetracycline operator sequence (tetO), and a tetracycline transactivator fusion protein (tTA), and a tetracycline operator sequence (tetO) and a reverse tetracycline transactivator fusion protein (rtTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters. A non-limiting example of an inducible system that uses a light-regulated promoter is provided in Wang et al., Nat. Methods. 2012 Feb. 12; 9(3):266-9.

As used herein, a “TRE promoter” is a promoter comprising a tetracycline-responsive element (TRE). As used herein, a TRE comprises at least one (e.g., at least 2, 3, 4, 5, 6, 7, 8, 9, 10, 11, 12, 13, 14, 15, 16, 17, 18, 19, or 20) Tet-O sequences. A non-limiting example of a Tet-O sequence is sequence that is at least 70% (e.g., 75%, 80%, 85%, 90%, 95%, 98%, 99%, or 100%) identical to SEQ ID NO: 19. In some embodiments, a TRE promoter further comprises a minimal promoter located downstream of a tet-O sequence. A minimal promoter is a promoter that comprises the minimal elements of a promoter (e.g., TATA box and transcription initiation site), but is inactive in the absence of an upstream enhancer (e.g., sequences comprising Tet-O). As an example, a minimal promoter may be a minimal CMV promoter that comprises a sequence that is at least 70% (e.g., at least 75%, 80%, 85%, 90%, 95%, 98%, 99%, or 100%) identical to SEQ ID NO: 20. For example, a TRE promoter may be a TRE3G promoter (e.g., a TRE3G promoter that comprises a sequence that is at least 70% (e.g., at least 75%, 80%, 85%, 90%, 95%, 98%, 99%, or 100%) identical to SEQ ID NO: 7. In some embodiments, a TRE promoter is a TRE2 promoter comprising a sequence that is at least 70% (e.g., at least 75%, 80%, 85%, 90%, 95%, 98%, 99%, or 100%) identical to SEQ ID NO: 23. In some embodiments, a TRE promoter is a P tight promoter comprising a sequence that is at least 70% (e.g., at least 75%, 80%, 85%, 90%, 95%, 98%, 99%, or 100%) identical to SEQ ID NO: 24.

A “reverse tetracycline transactivator” (“rtTA”), as used herein, is an inducing agent that binds to a TRE promoter (e.g., a TRE3G, P tight, or TRE2 promoter) in the presence of tetracycline (e.g., doxycycline) and is capable of driving expression of a transgene that is operably linked to the TRE promoter. rtTAs generally comprise a mutant tetracycline repressor DNA binding protein (TetR) and a transactivation domain (see, e.g., Gossen et al., Science. 1995 Jun. 23; 268(5218):1766-9. Any suitable transactivation domain may be used. Non-limiting examples include VP64, P65, RTA, and MPH MS2-P65-HSF1. In some embodiments, a rtTA of the present disclosure comprises at least 1, 2, 3, 4, 5, 6, 7, 8, 9, 10, 11, 12, 13, 14, 15, 16, 17, 18, 19, 20, 30, 40, 50, 60, 70, 80, 90, or 100 transactivation domains. The mutant TetR domain is capable of binding to a TRE promoter when bound to tetracycline.

The rtTA may be rtTA3, rtTA4, or variants thereof. As used herein, a rtTA3 amino acid sequence comprises the following amino acids at a position corresponding to the residue in SEQ ID NO: 11: glycine at a residue 72, glycine at residue 12, phenylalanine at residue 67, and arginine at residue 171. In certain embodiments, a nucleic acid encoding rtTA3 comprises a sequence that is at least 70% (e.g., at least 75%, at least 80%, at least 85%, at least 90%, at least 95%, or at least 100%) identical to SEQ ID NO: 10 and/or the nucleic acid sequence encodes a rtTA3 protein that comprises the following amino acids at a position corresponding to the residue in SEQ ID NO: 11: glycine at a residue 72, glycine at residue 12, phenylalanine at residue 67, and arginine at residue 171. A rtTA3 nucleotide sequence may consist of SEQ ID NO: 10. In certain embodiments, an amino acid sequence encoding rtTA3 comprises a sequence that is at least 70% (e.g., at least 75%, at least 80%, at least 85%, at least 90%, at least 95%, at least 98%, at least 99%, or at least 100%) identical to (SEQ ID NO: 11), and comprises the following amino acids corresponding to the indicated positions in SEQ ID NO: 11: glycine at residue 72, glycine at residue 12, phenylalanine at residue 67, and arginine at residue 171. A rtTA3 amino acid sequence may consist of SEQ ID NO: 11.

As used herein, a rtTA4 amino acid sequence comprises mutations at positions corresponding to the following residues in SEQ ID NO: 11: G72; G12; F67; and R171. In certain embodiments, G72; G12; F67; and R171 may be mutated to any residue. In certain embodiments, a nucleic acid encoding rtTA4 comprises a sequence that is at least 70% (e.g., at least 75%, at least 80%, at least 85%, at least 90%, at least 95%, at least 98%, at least 99%, or at least 100%) identical to SEQ ID NO: 12 and encodes a protein with a mutation at the following residues corresponding to the following positions in rtTA3 (SEQ ID NO: 11): G72; G12; F67; and R171. A rtTA4 nucleic acid sequence may consist of SEQ ID NO: 12.

In certain embodiments, an amino acid sequence encoding rtTA4 comprises a sequence that is at least 70% (e.g., at least 75%, at least 80%, at least 85%, at least 90%, at least 95%, at least 98%, at least 99%, or at least 100%) identical to SEQ ID NO: 13 and/or encodes a protein with a mutation at the following residues corresponding to the following positions in rtTA3 (SEQ ID NO: 11): G72; G12; F67; and R171. In certain embodiments, a rtTA4 amino acid sequence comprises the following mutations relative to rtTA3: a valine (V) or proline (P) mutation at a residue corresponding to position G72 in SEQ ID NO: 11, a serine (S) mutation at a residue corresponding to position G12 in SEQ ID NO: 11, a serine (S) mutation at a residue corresponding to position F67 in SEQ ID NO: 11, and an lysine (K) mutation at a residue corresponding to position R171 in SEQ ID NO: 11. A rtTA4 amino acid sequence may consist of SEQ ID NO: 13.

A “multicistronic vector” is a vector that encodes more than one amino acid sequence (e.g., a vector encoding at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 proteins). A multicistronic vector allows for expression of multiple amino acid sequences from a nucleic acid sequence. Nucleic acid sequences encoding each protein may be connected or separated such that they produce unconnected proteins. For example, internal ribosome entry sites (IRES) or polypeptide cleavage signals may be placed between nucleic acid sequences encoding each transcription factor in a vector. Exemplary polypeptide cleavage signals include 2A peptides (e.g., T2A, P2A, E2A, and F2A). A 2A peptide may comprise a sequence that is at least 70% (e.g., at least at least 75%, 80%, 85%, 90%, 95%, 98%, 99%, or 100%) identical to SEQ ID NO: 9. In some embodiments, a nucleic acid (e.g., an engineered nucleic acid, including an expression vector) of the present disclosure is a multicistronic expression vector.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

A “prokaryotic cell” is a cell that lacks a membrane-bound organelle. Non-limiting examples of prokaryotes include archaea and bacteria.

“Reversing aging,” as used herein, refers to modifying the physical characteristics associated with aging. All animals typically go through a period of growth and maturation followed by a period of progressive and irreversible physiological decline ending in death. The length of time from birth to death is known as the life span of an organism, and each organism has a characteristic average life span. Aging is a physical manifestation of the changes underlying the passage of time as measured by percent of average life span.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds, such as chickens, ducks, geese, and/or turkeys). In certain embodiments, the animal is a mammal. The animal may be a male or female and at any stage of development. A non-human animal may be a transgenic animal.

A “terminator” or “terminator sequence,” as used herein, is a nucleic acid sequence that causes transcription to stop. A terminator may be unidirectional or bidirectional. It is comprised of a DNA sequence involved in specific termination of an RNA transcript by an RNA polymerase. A terminator sequence prevents transcriptional activation of downstream nucleic acid sequences by upstream promoters. Thus, in certain embodiments, a terminator that ends the production of an RNA transcript is contemplated.

The most commonly used type of terminator is a forward terminator. When placed downstream of a nucleic acid sequence that is usually transcribed, a forward transcriptional terminator will cause transcription to abort. In some embodiments, bidirectional transcriptional terminators may be used, which usually cause transcription to terminate on both the forward and reverse strand. In some embodiments, reverse transcriptional terminators may be used, which usually terminate transcription on the reverse strand only.

Non-limiting examples of mammalian terminator sequences include bovine growth hormone terminator, and viral termination sequences such as, for example, the SV40 terminator, spy, yejM, secG-leuU, thrLABC, rrnB T1, hisLGDCBHAF1, metZWV, rrnC, xapR, aspA, and arcA terminator. In certain embodiments, the terminator sequence is SV40 and comprises a sequence that is at least 70% (e.g., at least 75%, 80%, 85%, 90%, 95%, 98%, 99%, or 100%) identical to SEQ ID NO: 8.

A “Tet-Off” system, as used herein, is a type of inducible system that is capable of repressing expression of a particular transgene in the presence of tetracycline (e.g., doxycycline (DOX)). Conversely, a Tet-Off system is capable of inducing expression of a particular transgene in the absence of tetracycline (e.g., doxycycline, DOX). In certain embodiments, a Tet-Off system comprises a tetracycline-responsive promoter operably linked to a transgene (e.g., encoding a protein, a gene-targeting nucleic acid, and/or a therapeutic gene) and a tetracycline-controlled transactivator (tTA). The transgene with the tetracycline-responsive promoter (e.g., TRE3G, a TRE2, or a P tight promoter) and the tetracycline-controlled transactivator may be encoded on the same vector or be encoded on separate vectors.

A “Tet-On” system, as used herein, is a type of inducible system that is capable of inducing expression of a particular transgene in the presence of tetracycline (e.g., doxycycline (DOX)). In certain embodiments, a Tet-On system comprises a tetracycline-responsive promoter operably linked to a transgene (e.g., a therapeutic sequence, a gene-targeting nucleic acid, and/or a nucleic acid encoding a protein) and a reverse tetracycline-controlled transactivator (rtTA).

The expression cassette encoding a tetracycline-responsive promoter (e.g., a promoter comprising a TRE, including TRE3G, P tight, and TRE2) and a reverse tetracycline-controlled transactivator may be encoded on the same vector or be encoded on separate vectors.

The term “tetracycline repressor” or “TetR” refers to a protein that is capable of binding to a Tet-O sequence (e.g., a Tet-O sequence in a TRE) in the absence of tetracycline (e.g., doxycycline) and prevents binding of rtTA (e.g., rtTA3, rtTA4, or variants thereof) in the absence of tetracycline (e.g., doxycycline). TetRs prevent gene expression from promoters comprising a TRE in the absence of tetracycline (e.g., doxycycline). In the presence of tetracycline, TetRs cannot bind promoters comprising a TRE, and TetR cannot prevent transcription. Non-limiting examples of TetRs include tetR (e.g., SEQ ID NO: 26), and tetRKRAB (e.g., SEQ ID NO: 28). In some embodiments, a TetR is a TetR fusion (e.g., TRSID, which may be created by fusing TetR to a mSIN30interacting domain (SID) of Mad1). See, e.g., Zhang et al., J Biol Chem. 2001 Nov. 30; 276(48):45168-74.

The term “therapeutic sequence” as used herein is any transgene that encodes a therapeutic nucleic acid and/or protein, (including prophylactic nucleic acids and/or proteins and diagnostic nucleic acids and/or proteins). For example, a nonlimiting list of transgene sequences that are therapeutic sequences is described in O'Connor et al., Nat Rev Genet. 2006 April; 7(4):261-76. Non-limiting examples of therapeutic proteins include antibodies, enzymes, kinases, hormones, growth factors, cytokines, plasma proteins, fusion proteins, membrane-lytic proteins and coagulation factors. In some embodiments, a therapeutic protein is an inflammatory agent. In some embodiments, a therapeutic protein is an anti-inflammatory agent. In some embodiments, a therapeutic protein is an immunomodulatory agent. In some embodiments, a therapeutic protein is an anti-cancer agent. In some embodiments, a therapeutic protein is a metabolic agent. In some embodiments, a therapeutic protein is an antiviral/virocidal agent. In some embodiments, a therapeutic protein is an antibacterial/bacteriocidal agent.

The term “tissue” refers to any biological tissue of a subject (including a group of cells, a body part, or an organ) or a part thereof, including blood and/or lymph vessels, which is the object to which a compound, particle, and/or composition of the invention is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is considered healthy but suboptimal for performance or survival in current or future conditions. For example, in agricultural practice, environmental conditions including weather and growing conditions (e.g., nutrition) may benefit from any of the methods described herein. In certain embodiments, the tissue is the central nervous system. In certain embodiments, the tissue refers to tissue from the In certain embodiments, the cell or tissue is from eye, ear, nose, mouth including gum and roots of teeth, bone, lung, breast, udder, pancreas, stomach, oesophagus, muscle including cardiac muscle, liver, blood vessel, skin including hair, heart, brain, nerve tissue, kidney, testis, prostate, penis, cloaca, fin, ovary, or intestine. In certain embodiments, the tissue is damaged (e.g., due to a congenital defect, an injury, an accident, or an iatrogenic injury) and/or is aged tissue. In certain embodiments, the tissue is a deep tissue that is reachable with a fiber optic probe.

The term “tissue repair” in the context of damaged tissue refers to restoration of tissue architecture, function following tissue damage, or a combination thereof. Tissue repair includes tissue regeneration, cell growth, tissue replacement, and/or rewiring of existing tissue (reprogramming).

The term “tissue regeneration” refers to production of new tissue or cells within a tissue that are the same type as the tissue of interest (e.g., same type as the damaged tissue or cell). In some embodiments, the methods provided herein promote organ regeneration.

The term “tissue replacement” refers to production of a different type of tissue compared to the tissue of interest (e.g., connective tissue to replace damaged tissue).

As used herein, the terms “treatment,” “treat,” and “treating” refer to reversing, alleviating, delaying the onset of, or inhibiting the progress of a disease or disorder, or one or more symptoms thereof, as described herein. In certain embodiments, treatment may be administered after one or more symptoms have developed. In other embodiments, treatment may be administered in the absence of symptoms. For example, treatment may be administered to a susceptible individual prior to the onset of symptoms or may be treated with another damaging agent (e.g., in light of a history of symptoms, in light of genetic or other susceptibility factors, a disease therapy or any combination thereof). Treatment may also be continued after symptoms have resolved, for example, to prevent or delay their recurrence.

The term “variant” or “mutant” refers to a sequence that comprises a modification relative to a wild-type sequence (e.g., rtTA3 sequence). Non-limiting modifications in an amino acid sequence include insertions, deletions, truncation mutations and point mutations. Non-limiting modifications to nucleic acid sequences include frameshift mutations, nucleotide insertions, and nucleotide deletions.

The term “WPRE” refers to a Woodchuck Hepatitis Virus (WHP) Posttranscriptional Regulatory Element (WPRE). WPREs create tertiary structures in nucleic acids (e.g., expression vectors) and are capable of enhancing transgene expression (e.g., from a viral vector). In certain embodiments, a WPRE sequence is at least 70% (e.g., at least 75%, at least 80%, at least 85%, at least 90%, at least 95%, at least 98%, at least 99%, or at least 100%) identical to SEQ ID NO: 21.

These and other exemplary substituents are described in more detail in the Detailed Description, Examples, and claims. The invention is not intended to be limited in any manner by the above exemplary listing of substituents.

### BRIEF DESCRIPTION OF THE DRAWINGS

FIG. 1 is a vector map showing features in an adeno-associated virus (AAV) vector encoding reverse tetracycline-transactivator 4 (rtTA4). Ubc is a constitutive promoter that is operably linked to the nucleic acid encoding rtTA4. SV40 pA is an SV40-derived terminator sequence. The sequence of this vector is provided in SEQ ID NO: 17.

FIGS. 2A-2M include a series of schematics mapping the features shown in FIG. 1 onto the nucleic acid sequence of the vector encoding rtTA4 (SEQ ID NOs: 17-28, top and bottom rows respectively).

FIG. 3 shows the location and size of each feature depicted in FIGS. 2A-2M.

FIGS. 4A-4B include data showing that a tetracycline-on (Tet-On) system with rtTA3 (SEQ ID NO: 11) as the inducing agent is leaky in vivo. FIG. 4A is a non-limiting example of a Tet-On system comprising two nucleic acids. The first nucleic acid comprises a UBC promoter operably linked to sequences that encode rtTA3, a self-cleaving peptide (2A peptide), and mKate (far-red fluorescent protein) (FIG. 4A, top). The second nucleic acid encodes an inducible promoter with a tetracycline-responsive element (TRE) (TRE3G promoter, SEQ ID NO: 7) that is operably linked to GFP (FIG. 4A, bottom). DOX indicates doxycycline. FIG. 4B shows a western blot of liver samples from mice that were treated with (1) no AAVs, (2) AAVs harboring an AAV vector that comprises the first nucleic acid in FIG. 4A, and AAVs harboring an AAV vector that comprises the second nucleic acid in FIG. 4A in the absence of doxycycline (DOX), or (3) the AAVs of (2) in the presence of doxycycline (DOX). mKate, GFP, and actin expression is shown.

FIGS. 5A-5B show that a tetracycline-on (Tet-On) system with rtTA4 (SEQ ID NO: 13) is less leaky than the same system with rtTA3. FIG. 5A is a schematic depicting a Tet-On system reporter system with rtTA4. The top portion of the schematic shows a nucleic acid, in which a promoter sequence operably linked to a sequence encoding rtTA4. The bottom portion of the schematic shows a second nucleic acid, in which a TRE3G promoter is operably linked to a sequence encoding luciferase. FIG. 5B is a graph showing the effect of doxycycline (DOX, ng/ml) on the production of luciferase in a Tet-On reporter system using rtTA3 compared to a Tet-On reporter system using rtTA4. Luciferase production is measured in luminescence/protein. The baseline level of luminescence/protein in the absence of any rtTA is shown.

FIGS. 6A-6C include a series of graphs showing that a Tet-On system with rtTA4 turns off faster in response to doxycycline withdrawal compared to a Tet-On system with rtTA3. A DOX-inducible luciferase reporter system was used, and luciferase production was measured in luminescence/protein. +DOX indicates doxycycline treatment, −DOX indicates absence of doxycycline treatment, +−DOX indicates doxycycline treatment followed by doxycycline withdrawal. FIG. 6A is a graph showing the effect of doxycycline on expression in a Tet-On reporter system, in which rtTA3 expression is driven by a Desmin promoter and luciferase expression is controlled by a TRE3G promoter. FIG. 6B is a graph showing the effect of doxycycline on expression in a Tet-On reporter system, in which with rtTA4 expression is driven by a Desmin promoter and luciferase expression is controlled by a TRE3G promoter. FIG. 6C is a graph showing the length of time for doxycycline withdrawal needed to reduce luciferase expression in a Tet-On reporter system with rtTA3 compared to that in a Tet-On reporter system with rtTA4. In both reporter systems, expression of the rtTA is driven by a UBC promoter, and expression of luciferase is controlled by a TRE3G promoter.

FIG. 7 is a western blot comparing the effects of doxycycline treatment and withdrawal on transgene expression induced by rtTA3 (SEQ ID NO: 11) and rtTA4 (SEQ ID NO: 13) in 293T cells. The nucleic acid sequence (SEQ ID NO: 10) encoding rtTA3 was operably linked to a UBC promoter (SEQ ID NO: 18), while rtTA4 was operably linked to a UBC promoter (SEQ ID NO: 18) or a desmin promoter (SEQ ID NO: 29).

FIGS. 8A-8C include data showing that a Tet-On system comprising rtTA4 (SEQ ID NO: 13) has low leakiness in the liver of mice. FIG. 8A is a series of immunofluorescence images showing expression of KLF4 in the livers of mice that have been administered AAVs harboring nucleic acids shown in FIG. 8B in the absence of doxycycline (no DOX) and in the presence of doxycycline (with DOX). DAPI is a nuclear stain that was used to visualize cells.

FIG. 8B is a schematic depicting the two nucleic acids that were administered to mice in AAV9 viruses. FIG. 8C is a western blot of liver samples from mice that received the constructs depicted in FIG. 8B and were treated with no doxycycline or with doxycycline. OCT4, KLF4, and SOX2 levels were detected as indicated using antibodies. Actin is shown as a loading control.

FIG. 9 is a vector map depicting a non-limiting example of an inducible AAV vector (TRE3G-OSK-SV40 pA, SEQ ID NO: 16) encoding Oct4, SOX2, and KLF4, which may be used in combination with any of the rtTA4 vectors described herein.

FIG. 10 is a schematic showing a non-limiting example of an inducible expression system combining an rtTA (e.g., rtTA4) and a tetracycline repressor (tetR, e.g., tetRKRAB). The triangle represents tetR (e.g., tetRKRAB) protein and the circle represents rtTA4 protein.

FIG. 11 is a vector map of pAAV2_CMV_rtTA(VP16) (SEQ ID NO: 31). This vector is a non-limiting example of a vector encoding rtTA.

FIG. 12 is a vector map of pAAV-MCS-tTA2 (or CAG-tTA) (SEQ ID NO: 32). This vector is a non-limiting example of a vector encoding tTA under a CAG promoter.

FIG. 13 is a vector map of p-AAV-TetO-OSK-WPRE3-SV50LpA (TRE2-OSK, pAAV-TRE2-OSK-SV40LpA, or TRE2-OSK) (SEQ ID NO: 33). This vector is a non-limiting example of an AAV vector comprising a nucleic acid (e.g., engineered nucleic acid) sequence that is greater than 4.7 kb between the two ITRs in the vector.

FIG. 14 shows that partial reprogramming with AAV-delivered polycistronic OSK under the control of rtTA4 is non-toxic. FIG. 14 shows body weight of WT mice, OSK transgenic mice, and AAV-mediated OSK-expressing mice (1.0×10¹² gene copies) with or without doxycycline induction in the first 4 weeks (n=5, 3, 6, 4, 6, 3, respectively).

FIGS. 15A-15E shows that rtTA4 may be used in vivo to control OSK expression. FIG. 15A shows AAV9 expression in the liver compared to transgenic mice. FIG. 15B shows the body weight of WT mice and AAV-mediated OSK-expressing mice (1.0×10\hat{ }12 gene copies total) with or without doxycycline in the following 9 months after first 4 weeks (n=5, 3, 6, 4, respectively). FIG. 15C shows AAV-UBC-rtTA and AAV-TRE-Luc vectors used for measuring tissue distribution. FIG. 15D shows luciferase imaging of WT mice at 2 months after retro-orbital injections of AAV9-UBC-rtTA and AAV9-TRE-Luc (1.0×10\hat{ }12 gene copies total). Doxycycline was delivered in drinking water (1 mg/mL) for 7 days to the mouse shown on the right. FIG. 15E shows luciferase imaging of eye (Ey), brain (Br), pituitary gland (Pi), heart (He), thymus (Th), lung (Lu), liver (Li), kidney (Ki), spleen (Sp), pancreas (Pa), testis (Te), adipose (Ad), muscle (Mu), spinal cord (SC), stomach (St), small intestine (In), and cecum (Ce) 2 months after retro-orbital injection of AAV9-UBC-rtTA and AAV9-TRE-Luc followed by treatment with doxycycline for 7 days. The luciferase signal is primarily in liver. Imaging the same tissues with a longer exposure time (FIG. 15E, lower panel) revealed lower levels of luciferase signal in pancreas (liver was removed).

### DETAILED DESCRIPTION OF CERTAIN EMBODIMENTS OF THE INVENTION

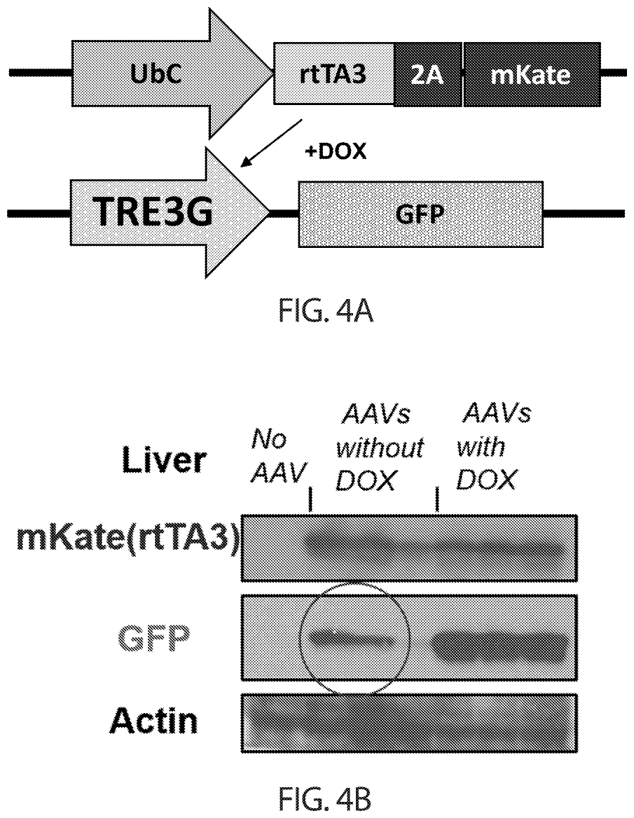

The present disclosure is based, at least in part, on the unexpected results demonstrating that four mutations corresponding to residues G72, G12, F67, and R171 in rtTA3 (SEQ ID NO: 11) produced a mutant rtTA (referred to herein as rtTA4) that had lower leakiness as compared to rtTA3 and improved sensitivity to tetracycline withdrawal. Whereas existing rtTA3 Tet-On systems are leaky (FIG. 4B) in vivo (e.g., in the liver of mice), the rtTA4 system described herein did not induce detectable transgene expression (e.g., in the livers of mice).

As shown herein, a rtTA4 sequence (e.g., SEQ ID NO: 13) was less leaky than rtTA3 (SEQ ID NO: 11), and in the absence of a tetracycline (e.g., doxycycline), rtTA4 did not induce detectable transgene expression in the liver, whereas rtTA3 induced transgene expression, even in the absence of a tetracycline (e.g., doxycycline). Furthermore, a Tet-On system with rtTA4 turned off 4-12 times faster than rtTA3. In certain embodiments, the rtTA4 Tet-On system described herein is further regulated by a tetR (tetRKRAB), which can bind to the TRE promoter in the absence of a tetracycline (e.g., doxycycline), preventing binding of rtTA and thereby further repressing gene expression.

Accordingly, provided herein, in some embodiments, are mutant rtTAs (e.g., rtTA4), engineered nucleic acids (e.g., expression vectors), recombinant viruses, systems, kits, and compositions comprising the same and methods of regulating gene expression using the same. Any of the mutant rtTAs, nucleic acids (e.g., engineered nucleic acids, including expression vectors) comprising a mutant rtTA, recombinant viruses, systems, kits, and compositions comprising the same may be useful in regulating gene expression, inducing cellular reprogramming, tissue repair, tissue regeneration, organ regeneration, reversing aging, treating a disease (e.g., acute injuries, neurodegenerative disease, chronic diseases, proliferative diseases, cardiovascular diseases, genetic diseases, inflammatory diseases, autoimmunue diseases, neurological diseases, hematological diseases, painful conditions, psychiatric disorders, metabolic disorders, cancers, aging, age-related diseases, and diseases affecting any tissue in a subject), or any combination thereof

Mutant Reverse Tetracycline Transactivators (rtTAs)

Aspects of the present disclosure provide mutant reverse tetracycline transactivators (rtTAs) that are capable of activating expression of genes from an operably linked tetracycline-responsive element (TRE) (e.g., a TRE3G, a TRE2, or a P tight promoter) in the presence of a tetracycline (e.g., doxycycline).

A mutant rtTA of the present disclosure comprises at least 1, 2, 3, 4, 5, 6, 7, 8, 9, 10, 11, 12, 13, 14, 15, 16, 17, 18, 19, 20, 30, 40, 50, 60, 70, 80, 90, or 100 transactivation domains. Non-limiting examples of transactivation domains include VP64, P65, RTA and MPH MS2-P65-HSF1. In some embodiments, a nucleotide sequence encoding VP64 comprises a sequence that is at least 70% (e.g., at least 75%, 80%, 85%, 90%, 95%, 98%, 99%, or 100%) identical to SEQ ID NO: 34. In some embodiments, an amino acid sequence encoding VP64 comprises a sequence that is at least 70% (e.g., at least 75%, 80%, 85%, 90%, 95%, 98%, 99%, or 100%) identical to the amino acid sequence encoded by SEQ ID NO: 34. In some embodiments, a nucleotide sequence encoding P65 comprises a sequence that is at least 70% (e.g., at least 75%, 80%, 85%, 90%, 95%, 98%, 99%, or 100%) identical to SEQ ID NO: 35. In some embodiments, an amino acid sequence encoding P65 comprises a sequence that is at least 70% (e.g., at least 75%, 80%, 85%, 90%, 95%, 98%, 99%, or 100%) identical to the amino acid sequence encoded by SEQ ID NO: 35. In some embodiments, a nucleotide sequence encoding RTA comprises a sequence that is at least 70% (e.g., at least 75%, 80%, 85%, 90%, 95%, 98%, 99%, or 100%) identical to SEQ ID NO: 36. In some embodiments, an amino acid sequence encoding RTA comprises a sequence that is at least 70% (e.g., at least 75%, 80%, 85%, 90%, 95%, 98%, 99%, or 100%) identical to the amino acid sequence encoded by SEQ ID NO: 36. In some embodiments, a nucleotide sequence encoding MPH MS2-P65-HSF1 comprises a sequence that is at least 70% (e.g., at least 75%, 80%, 85%, 90%, 95%, 98%, 99%, or 100%) identical to SEQ ID NO: 37. In some embodiments, an amino acid sequence encoding MPH MS2-P65-HSF1 comprises a sequence that is at least 70% (e.g., at least 75%, 80%, 85%, 90%, 95%, 98%, 99%, or 100%) identical to the amino acid sequence encoded by SEQ ID NO: 37.

The mutant rtTAs (e.g., rtTA4) of the present disclosure comprise mutations at positions corresponding to the following residues in SEQ ID NO: 11: G72; G12; F67; and R171. SEQ ID NO: 11 is a non-limiting example of a rtTA3 sequence. Non-limiting examples of mutations include point mutations, truncation mutations, deletions or insertions.

Non-limiting mutations at positions corresponding to residue G12 in SEQ ID NO: 11 include alanine (A), arginine (R), asparagine (N), aspartic acid (D), cysteine (C), glutamine (Q), glutamic acid (E), histidine (H), isoleucine (I), lysine (K), methionine (M), phenylalanine (F), proline (P), serine (S), threonine (T), tryptophan (W), tyrosine (Y), and valine (V).

Non-limiting mutations at positions corresponding to residue G72 in SEQ ID NO: 11 include alanine (A), arginine (R), asparagine (N), aspartic acid (D), cysteine (C), glutamine (Q), glutamic acid (E), histidine (H), isoleucine (I), lysine (K), methionine (M), phenylalanine (F), proline (P), serine (S), threonine (T), tryptophan (W), tyrosine (Y), and valine (V).

Non-limiting mutations at positions corresponding to residue F67 in SEQ ID NO: 11 include alanine (A), arginine (R), asparagine (N), aspartic acid (D), cysteine (C), glutamine (Q), glutamic acid (E), glycine (G), histidine (H), isoleucine (I), lysine (K), methionine (M), proline (P), serine (S), threonine (T), tryptophan (W), tyrosine (Y), and valine (V).

Non-limiting mutations at positions corresponding to residue F67 in SEQ ID NO: 11 include alanine (A), asparagine (N), aspartic acid (D), cysteine (C), glutamine (Q), glutamic acid (E), glycine (G), histidine (H), isoleucine (I), lysine (K), methionine (M), proline (P), phenylalanine (F), serine (S), threonine (T), tryptophan (W), tyrosine (Y), and valine (V).

Non-limiting mutations at positions corresponding to residue F67 in SEQ ID NO: 11 include alanine (A), arginine (R), asparagine (N), aspartic acid (D), cysteine (C), glutamine (Q), glutamic acid (E), glycine (G), histidine (H), isoleucine (I), lysine (K), methionine (M), proline (P), serine (S), threonine (T), tryptophan (W), tyrosine (Y), and valine (V).

The mutation at G72; G12; F67; and/or R171 may be a mutation to an amino acid with a charged side chain (e.g., arginine, histidine, lysine, aspartic acid, or glumatic acid). The amino acid may comprise a negatively charged side chain (e.g., aspartic acid or glutamic acid). The amino acid may comprise a positively charged side chain (arginine, histidine or lysine).

The mutation at G72; G12; F67; and/or R171 may be a mutation to an amino acid comprising a polar uncharged side chain (e.g., serine, threonine, asparagine, glutamine) or a comprising a hydrophobic side chain (e.g., alanine, valine, isoleucine, leucine, methionine, phenylalanine, tyrosine, or tryptophan).

The mutation at G72; G12; F67; and/or R171 may be to an amino acid comprising an aromatic ring (e.g., phenylalanine, tyrosine, or tryptophan).

In certain embodiments, a nucleic acid encoding rtTA4 comprises a sequence that is at least 70% (e.g., at least 75%, at least 80%, at least 85%, at least 90%, at least 95%, at least 98%, at least 99%, or at least 100%) identical to SEQ ID NO: 12 and/or encodes a protein with a mutation at the following residues corresponding to the following positions in rtTA3 (SEQ ID NO: 11): G72; G12; F67; and R171. A rtTA4 nucleic acid sequence may consist of SEQ ID NO: 12.

In certain embodiments, an amino acid sequence encoding rtTA4 comprises a sequence that is at least 70% (e.g., at least 75%, at least 80%, at least 85%, at least 90%, at least 95%, at least 98%, at least 99%, or at least 100%) identical to SEQ ID NO: 13 and/or encodes a protein with mutations at the following residues corresponding to the following positions in rtTA3 (SEQ ID NO: 11): G72; G12; F67; and R171.

In certain embodiments, a mutant rtTA comprises at least another mutation in a position corresponding to rtTA3 (SEQ ID NO: 11) in addition to the mutations corresponding to residues G72; G12; F67; and R171 in SEQ ID NO: 11. In certain embodiments, a mutant rtTA comprises at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 80, 90, 100, 110, 120, 130, 140, 150, 160, 170, 180, 190, 200, 210, 220, 230, or 240 additional mutations corresponding to a position that is not residues G72, G12, F67 and R171 in SEQ ID NO: 11.

It should be appreciated that any rtTA (e.g., rtTA3 or M2-rtTA) could be used as the reference sequence to be mutated. For example, a mutant rtTA of the present disclosure may comprise mutations corresponding to residues G72; G12; F67; and R171 of SEQ ID NO: 11, but otherwise comprise amino acid sequences present in M2-rtTA. In certain embodiments, in addition to the mutations corresponding to residues G72; G12; F67; and R171 of SEQ ID NO: 11, a rtTA of the present disclosure may comprise at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 80, 90, 100, 110, 120, 130, 140, 150, 160, 170, 180, 190, 200, 210, 220, 230, or 240 additional mutations corresponding to positions that are not residues G72, G12, F67 and R171 in SEQ ID NO: 11.

In certain embodiments, a rtTA4 amino acid sequence comprises the following mutations relative to rtTA3: a valine (V) or proline (P) mutation at a residue corresponding to position G72 in SEQ ID NO: 11, a serine (S) mutation at a residue corresponding to position G12 in SEQ ID NO: 11, a serine (S) mutation at a residue corresponding to position F67 in SEQ ID NO: 11, and an lysine (K) mutation at a residue corresponding to position R171 in SEQ ID NO: 11. A rtTA4 amino acid sequence may consist of SEQ ID NO: 13.

Any suitable sequence alignment algorithm may be used to align two sequences of interest (e.g., a rtTA4 of the present disclosure with rtTA3 set forth as SEQ ID NO: 11) to identify the corresponding residue position in rtTA3. As a non-limiting example, SEQ ID NO: 13 (amino acid sequence of rtTA4) may be aligned to SEQ ID NO: 11 (amino acid sequence of rtTA3) using Clustal Omega (see e.g., Larkin et al., Bioinformatics. 2007 Nov. 1; 23(21):2947-8). In the exemplary alignment below, position 12 (glycine) in SEQ ID NO: 11 is mutated to serine in SEQ ID NO: 13, position 72 (glycine) in SEQ ID NO: 11 is mutated to valine in SEQ ID NO: 13, position 67 (phenylalanine) in SEQ ID NO: 11 is mutated to serine in SEQ ID NO: 13, position 171 (arginine) in SEQ ID NO: 11 is mutated to lysine in SEQ ID NO: 13.

```
SEQ ID NO 11 MSRLDKSKVINGALELLNGVGIEGLTTRKLAQKLGVEQPTLYWHVKNKRALLDALPIEML 60
SEQ ID NO 13 MSRLDKSKVINSALELLNGVGIEGLTTRKLAQKLGVEQPTLYWHVKNKRALLDALPIEML 60
                          ***********.************************************************
SEQ ID NO 11 DRHHTHFCPLEGESWQDFLRNNAKSYRCALLSHRDGAKVHLGTRPTEKQYETLENQLAFL 120
SEQ ID NO 13 DRHHTHSCPLEVESWQDFLRNNAKSYRCALLSHRDGAKVHLGTRPTEKQYETLENQLAFL 120
                          ***** **** *************************************************
SEQ ID NO 11 CQQGFSLENALYALSAVGHFTLGCVLEEQEHQVAKEERETPTTDSMPPLLRQAIELFDRQ 180
SEQ ID NO 13 CQQGFSLENALYALSAVGHFTLGCVLEEQEHQVAKEERETPTTDSMPPLLKQAIELFDRQ 180
                          **************************************************.*********
SEQ ID NO 11 GAEPAFLFGLELIICGLEKQLKCESGGPTDALDDFDLDMLPADALDDFDLDMLPG----- 235
SEQ ID NO 13 GAEPAFLFGLELIICGLEKQLKCESGGPTDALDDFDLDMLPADALDDFDLDMLPADALDD 240
                          *****************************************************.
SEQ ID NO 11 -------- 235
SEQ ID NO 13 FDLDMLPG 248
```

It should be understood, that the present disclosure encompasses rtTA4 variants that comprise mutations at the following residues corresponding to the following positions in rtTA3 (SEQ ID NO: 11): G72; G12; F67; and R171. Such a rtTA4 variant may comprise a sequence that is at least 70%, 75%, 80%, 85%, 90%, 91%, 92%, 93%, 94%, 95%, 96%, 97%, 98%, 99%, but less than 100% sequence identical to SEQ ID NO: 13.

The term “sequence identity,” as known in the art, refers to a relationship between the sequences of two polypeptides or two polynucleotides, as determined by sequence comparison (alignment). In some embodiments, sequence identity is determined across the entire length of a mutant rtTA (e.g., rtTA4) sequence. In some embodiments, sequence identity is determined over a region (e.g., a stretch of amino acids or nucleotides such as 10, 20, 30, 40, 50, etc. amino acids or nucleotides) of a mutant rtTA (e.g., rtTA4).

Identity can also refer to the degree of sequence relatedness between two sequences as determined by the number of matches between strings of two or more residues (e.g., nucleotides or amino acid residues). Identity measures the percent of identical matches between the smaller of two or more sequences with gap alignments (if any) addressed by a particular mathematical model or computer program (e.g., “algorithms”).

Identity of related polypeptides or nucleic acid sequences can be readily calculated by any of the methods known to one of ordinary skill in the art. The “percent identity” of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the protein molecules of the invention. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct. 11; 7:539) may be used.

In some embodiments, a protein encoding a mutant rtTA (e.g., rtTA4) is fused to a protein trandusction domain. Without being bound by a particular theory, a protein transduction domain facilitate delivery of a cargo (e.g., a protein, nucleic acids, nanoparticles, viral particles, etc.) across cellular membranes. Protein transduction domains include cationic peptides, hydrophobic peptides, and/or a cell specific peptides. See, e.g., Zhou et al., Cell Stem Cell. 2009 May 8; 4(5):381-4; Zahid et al., Curr Gene Ther. 2012 October; 12(5):374-80.

In some embodiments, a protein is formulated in a nanoparticle for delivery. In some embodiments, a chitosan polymeric nanoparticle is loaded with mutant rtTA (e.g., rtTA4) protein. See, e.g., Tammam et al., Oncotarget. 2016 Jun. 21; 7(25):37728-37739.

Nucleic Acids (e.g., Engineered Nucleic Acids) Encoding a Mutant rtTA

Any of the nucleic acid sequences encoding a recombinant rtTAs (e.g., rtTA4) described herein may be cloned into an expression vector. A nucleic acid (e.g., engineered nucleic acid) of the present disclosure may be present on a viral or non-viral vector. Suitable non-viral vectors include, but are not limited to, plasmid DNA or RNA (e.g., mRNA). In some embodiments, plasmid DNA may be incorporated into a nanoparticle and/or fused to a protein transduction domain (PTD). See above.

As a non-limiting example, the engineered nucleic acids (e.g., expression vectors) of the present disclosure (e.g., RNA, including mRNA, or DNA (e.g., plasmid DNA) of the present disclosure may be formulated in a nanoparticle for delivery. See, e. g., Dong et al., Nano Lett. 2016 Feb. 10; 16(2):842-8. In some embodiments, the nanoparticle comprises acetylated galactose. See, e.g., Lozano-Torres et al., J Am Chem Soc. 2017 Jul. 5; 139(26):8808-8811. In some embodiments, the engineered nucleic acids (e.g., expression vectors) (e.g., RNA, including mRNA, or DNA) is electroporated or transfected into a cell. In certain embodiments, the engineered nucleic acids are delivered as a naked nucleic acid (e.g., naked DNA or naked RNA).

Nonlimiting examples of viral vectors include lentivirus, adenovirus, alphavirus, vaccinia virus, retrovirus, herpes virus, and AAV vectors. Any of the recombinant rtTAs using methods known in the art. Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012). See also, e.g., general techniques below. Non-limiting examples of elements on a nucleic acid (e.g., an engineered nucleic acid, including an expression vector) include promoters, nucleic acid sequences operably linked to a promoter (e.g., an open-reading frame), a terminator sequence, a separator sequence, or a WPRE sequence. A vector may comprise one or more of these elements.

In certain embodiments, the nucleic sequence encoding a mutant rtTA is codon-optimized, for example, for expression in a particular host cell. In certain embodiments the sequence encoding a mutant rtTA is codon-optimized for expression in a mammalian cell. In certain embodiments the sequence encoding a mutant rtTA is codon-optimized for expression in a human cell. In certain embodiments, a sequence encoding a mutant rtTA is at least 70% (e.g., 75%, 80%, 85%, 90%, 95%, 98%, 99%, or 100%) identical to SEQ ID NO: 12.

The engineered nucleic acids (e.g., expression vectors) encoding a mutant rtTA (e.g., rtTA4) of the present disclosure comprise a promoter that is operably linked to the nucleic acid encoding rtTA. The promoter may be a constitutive promoter (e.g., CP1, CMV, EF1a, SV40, PGK1, Ubc, human beta actin, CAG, Ac5, polyhedrin, TEF1, GDS, CaM3 5S, Ubi, H1, or U6 promoter). A Ubc promoter may comprise a sequence that is at least 70% (e.g., at least 75%, 80%, 85%, 90%, 95%, 98%, 99%, or 100%) identical to SEQ ID NO: 18.

To allow for tissue-specific expression of a mutant tTA, the promoter may be tissue-specific (e.g., an eye-specific promoter, a bone-specific promoter, a lung-specific promoter, a breast-specific promoter, a pancreas-specific promoter, a muscle-specific promoter, a liver-specific promoter, a skin-specific promoter, a heart-specific promoter, a brain-specific promoter, a nerve tissue-specific promoter, a kidney-specific promoter, a testes-specific promoter, an ovary-specific promoter, or an intestine-specific promoter). In certain embodiments, the muscle-specific promoter is a desmin promoter. In some embodiments, a desmin promoter comprises a sequence that is at least 70% (e.g., 75%, 80%, 85%, 90%, 95%, 98%, 99%, or 100%) identical to SEQ ID NO: 29.

As will be appreciated by a person of ordinary skill in the art, the promoter operably linked to a nucleic acid encoding a mutant rtTA may be selected based on the host cell for expression. For example, a mammalian promoter may be used to drive expression of a mutant rtTA in a mammalian cell. A eukaryotic promoter may be used to drive expression of rtTA in a eukaryotic cell. A prokaryotic promoter may be used to drive expression of rtTA in a prokaryotic cell. A tissue specific promoter may be used to express a mutant rtTA in a tissue of interest within a subject.

In certain embodiments, a nucleic acid (e.g., engineered nucleic acid) sequence encoding a mutant rtTA is codon optimized for expression in a particular host cell. For example, a nucleic acid (e.g., engineered nucleic acid) encoding a mutant rtTA may be optimized for expression in a eukaryotic cell (e.g., a mammalian cell) or a prokaryotic cell.

The nucleic acid (e.g., engineered nucleic acid, including an expression vector) encoding the mutant rtTA may further comprise a Woodchuck Hepatitis Virus (WHP) Posttranscriptional Regulatory Element (WPRE), which may be useful in enhancing transgene gene expression (e.g., from a viral vector). In certain embodiments, a WPRE sequence is at least 70% (e.g., at least 75%, at least 80%, at least 85%, at least 90%, at least 95%, at least 98%, at least 99%, or at least 100%) identical to SEQ ID NO: 21. In certain embodiments, a WPRE sequence is located downstream of the nucleic acid encoding a mutant rtTA (e.g., rtTA4).

A terminator sequence may be used to designate the end of a transcript (cause transcription to stop). Non-limiting examples of mammalian terminator sequences include bovine growth hormone terminator, and viral termination sequences such as, for example, the SV40 terminator, spy, yejM, secG-leuU, thrLABC, rrnB T1, hisLGDCBHAF1, metZWV, rrnC, xapR, aspA, and arcA terminator. In certain embodiments, the terminator sequence is SV40 and comprises a sequence that is at least 70% (e.g., at least 75%, 80%, 85%, 90%, 95%, 98%, 99%, or 100%) identical to SEQ ID NO: 8.

A nucleic acid (e.g., an engineered nucleic acid, including an expression vector) encoding a mutant rtTA (e.g., rtTA4) may further encode a tetracycline repressor (TetR), which prevents gene expression from promoters comprising a TRE in the absence of a tetracycline (e.g., doxycycline). In the presence of tetracycline, TetRs cannot bind promoters comprising a TRE, and TetR cannot prevent transcription. Non-limiting examples of TetRs include TetR, TRSID, and tetRKRAB. In some embodiments, a TetR (e.g., TetR) is encoded by a nucleic acid comprising a sequence that is at least 70% (e.g., at least 75%, at least 80%, at least 85%, at least 90%, at least 95%, at least 98%, at least 99%, or at least 100%) identical to SEQ ID NO: 25. In some embodiments, a TetR (e.g., TetR) comprises a sequence that is at least 70% (e.g., at least 75%, at least 80%, at least 85%, at least 90%, at least 95%, at least 98%, at least 99%, or at least 100%) identical to SEQ ID NO: 26. In some embodiments, a TetR (e.g., tetRKRAB) is encoded by a nucleic acid comprising a sequence that is at least 70% (e.g., at least 75%, at least 80%, at least 85%, at least 90%, at least 95%, at least 98%, at least 99%, or at least 100%) identical to SEQ ID NO: 27. In some embodiments, a TetR (e.g., tetRKRAB) comprises a sequence that is at least 70% (e.g., at least 75%, at least 80%, at least 85%, at least 90%, at least 95%, at least 98%, at least 99%, or at least 100%) identical to SEQ ID NO: 28. A TetR may decrease transgene expression from a promoter by at least 10%, at least 20%, at least 30%, at least 40%, at least 50%, at least 60%, at least 70%, at least 80%, at least 90%, or 100%) in the absence of a tetracycline (e.g., doxycycline) as compared to in the presence of a tetracycline. Gene expression may be measured using any suitable method including assessment of protein levels and RNA levels.

A nucleic acid (e.g., an engineered nucleic acid, including an expression vector) may further comprise a separator sequence (e.g., an IRES or a polypeptide cleavage signal). Exemplary polypeptide cleavage signals include 2A peptides (e.g., T2A, P2A, E2A, and F2A). A 2A peptide may comprise a sequence that is at least 70% (e.g., at least 75%, 80%, 85%, 90%, 95%, 98%, 99%, or 100%) identical to SEQ ID NO: 9. For expression vectors encoding more than one transgene (e.g., a mutant rtTA and a tetR (e.g., tetRKRAB), each transgene may be operably linked to a different promoter or to the same promoter. The transgenes may be separated (e.g., an IRES or a polypeptide cleavage signal) on the expression vector. Expression of the nucleic acid results in separate amino acid sequences encoding each protein of interest.

In some embodiments, a nucleic acid (e.g., an engineered nucleic acid, including an expression vector) encoding a mutant rtTA (e.g., rtTA4) may comprise a sequence that is at least 70% (e.g., 75%, 80%, 85%, 90%, 95%, 98%, 99%, or 100%) identical to pAAV-UBC-rtTA4-WPRE3-SV40 pA (SEQ ID NO: 17). In some embodiments, a nucleic acid (e.g., an engineered nucleic acid, including an expression vector) encoding a mutant rtTA (e.g., rtTA4) consists of SEQ ID NO: 17. In some embodiments, a nucleic acid (e.g., an engineered nucleic acid, including an expression vector) encoding a mutant rtTA (e.g., rtTA4) may comprise a sequence that is at least 70% (e.g., 75%, 80%, 85%, 90%, 95%, 98%, 99%, or 100%) identical to desmin-rtTA4 vector (SEQ ID NO: 30). In some embodiments, a nucleic acid (e.g., an engineered nucleic acid, including an expression vector) encoding a mutant rtTA (e.g., rtTA4) consists of SEQ ID NO: 30.

Vectors of the invention may further comprise a marker sequence for use in the identification of cells that have or have not been transformed or transfected with the vector. Markers include, for example, genes encoding proteins that increase or decrease either resistance or sensitivity to antibiotics (e.g., ampicillin resistance genes, kanamycin resistance genes, neomycin resistance genes, tetracycline resistance genes and chloramphenicol resistance genes) or other compounds, genes encoding enzymes with activities detectable by standard assays known in the art (e.g., β-galactosidase, luciferase or alkaline phosphatase), and genes that visibly affect the phenotype of transformed or transfected cells, hosts, colonies or plaques (e.g., green fluorescent protein). In some embodiments, the vectors used herein are capable of autonomous replication and expression of the structural gene products present in the DNA segments to which they are operably linked.

In some embodiments, the nucleic acid (e.g., an engineered nucleic acid, including an expression vector) encoding the rtTA is a viral vector (e.g., lentivirus vector, adenovirus vector, alphavirus vector, vaccinia virus vector, herpes virus vector, adeno-associated virus (AAV) vector). An AAV vector, as used herein, generally comprises ITRs flanking an expression cassette (e.g., a nucleic acid comprising a promoter sequence operably linked to a sequence encoding a mutant rtTA, including rtTA4).

In certain embodiments, the number of base pairs between two ITRs in an AAV vector of the present disclosure is less than 5 kilobases (kb) (e.g., less than 4.9 kb, less than 4.8 kb, less than 4.7 kb, less than 4.6 kb, less than 4.5 kb, less than 4.4 kb, less than 4.3 kb, less than 4.2 kb, less than 4.1 kb, less than 4 kb, less than 3.5 kb, less than 3 kb, less than 2.5 kb, less than 2 kb, less than 1.5 kb, less than 1 kb, or less than 0.5 kb). In certain embodiments, an AAV vector with a distance of less than 4.7 kb between two ITRs is capable of being packaged into virus at a titer of at least 0.5×10\hat{ }10 particle forming units per ml (pfu/ml), at least 1×10\hat{ }10 pfu/ml, at least 5×10\hat{ }10 pfu/ml, at least 1×10\hat{ }11 pfu/ml, at least 5×10\hat{ }11 pfu/ml, at least 1×10\hat{ }12 pfu/ml, at least 2×10\hat{ }12 pfu/ml, at least 3×10\hat{ }12 pfu/ml, at least 4×10\hat{ }12 pfu/ml, at least 5×10\hat{ }12 pfu/ml, at least 6×10\hat{ }12 pfu/ml, at least 7×10\hat{ }12 pfu/ml, at least 8×10\hat{ }12 pfu/ml, at least 9×10\hat{ }12 pfu/ml, or at least 1×10\hat{ }13 pfu/ml.

In certain embodiments, the infection efficiency of a recombinant virus (e.g., lentivirus, adenovirus, alphavirus, vaccinia virus, retrovirus, herpes virus, or AAV) harboring a mutant rtTA (e.g., rtTA4) vector of the present disclosure in cells (e.g., animal cells, including mammalian cells) is at least 20% (e.g., at least 30%, at least 40%, at least 50%, at least 60%, at least 70%, at least 80%, or at least 90%, or 100%).

In certain embodiments, a nucleic acid (e.g., an engineered nucleic acid, including an expression vector) of the present disclosure is at least 1 kilobase (kb) (e.g., at least 1 kb, 2 kb, 3 kb, 4 kb, 5 kb, 6 kb, 7 kb, 8 kb, 9 kb, 10 kb, 50 kb, or 100 kb). In certain embodiments, a nucleic acid (e.g., an engineered nucleic acid, including an expression vector) of the present disclosure is less than 10 kb (e.g., less than 9 kb, less 8 kb, less than 7 kb, less than 6 kb, less than 5 kb, less than 4 kb, less than 3 kb, less than 2 kb, or less than 1 kb).

### Inducible Expression Vectors Encoding a Transgene

Any of the mutant rtTAs described herein may be used to promote expression of a transgene that is operably linked to an inducible promoter. In certain embodiments, a nucleic acid (e.g., an engineered nucleic acid, including an expression vector) encoding a mutant rtTA also comprises the transgene operably linked to an inducible promoter. In certain embodiments, a nucleic acid (e.g., an engineered nucleic acid, including an expression vector) encoding a mutant rtTA is separate from a nucleic acid (e.g., an engineered nucleic acid, including an expression vector) encoding a transgene operably linked to the inducible promoter. A mutant rtTA may drive expression of at least 1 inducible promoter comprising a Tet-O sequence. A mutant rtTA may drive expression of at least 2, 3, 4, 5, 6, 7, 8, 9, 10, 20, 30, 40, 50, 60, 70, 80, 90, or 100 inducible promoter comprising a Tet-O sequence.

It should be appreciated that one inducible promoter comprising a Tet-O sequence may be operably linked to at least 1, 2, 3, 4, 5, 6, 7, 8, 9, 10, 11, 12, 13, 14, 15, 16, 17, 18, 19, 20, 30, 40, 50, 60, 70, 80, 90, or 100 transgene sequences.

Suitable expression vectors encoding a transgene for use with a mutant rtTA of the present disclosure comprise an inducible promoter that comprise at least one Tet-O sequence (e.g., a TRE promoter) (e.g., an inducible promoter may comprise at least 1, 2, 3, 4, 5, 6, 7, 8, 9, 10, 11, 12, 13, 14, 15, 16, 17, 18, 19, 20 30, 40, 50, 60, 70, 80, 90, or all Tet-O sequences). A TRE promoter of the present disclosure may comprise a sequence that is at least 70% (e.g., at least 75%, at least 80%, at least 85%, at least 90%, at least 95%, at least 98%, at least 99%, or 100%) identical to SEQ ID NO: 7. The TRE promoter often comprises a minimal promoter, which cannot promote transcription in the absence of upstream enhancers that are present in the TRE promoter. A minimal promoter may be a minimal CMV promoter (e.g., a sequence that is at least 70%, at least 75%, at least 80%, at least 85%, at least 90%, at least 95%, at least 98%, at least 99%, or 100% identical to SEQ ID NO: 20). For example, a TRE promoter may be a TRE3G promoter (e.g., a sequence that is at least 70% (e.g., at least 75%, at least 80%, at least 85%, at least 90%, at least 95%, at least 98%, at least 99%, or 100%) identical to SEQ ID NO: 7). For example, a TRE promoter may be a TRE3G promoter (e.g., a sequence that is at least 70% (e.g., at least 75%, at least 80%, at least 85%, at least 90%, at least 95%, at least 98%, at least 99%, or 100%) identical to SEQ ID NO: 7). In some embodiments, the TRE promoter is a TRE2 or P tight promoter. In some embodiments, a TRE promoter is a TRE2 promoter and comprises a sequence that is at least 70%, least 75%, at least 80%, at least 85%, at least 90%, at least 95%, at least 98%, at least 99%, or at least 100% identical to SEQ ID NO: 23. In some embodiments, a TRE promoter is a P tight promoter and comprises a sequence that is at least 70%, least 75%, at least 80%, at least 85%, at least 90%, at least 95%, at least 98%, at least 99%, or at least 100% identical to SEQ ID NO: 24.

The inducible expression vectors of the present disclosure comprise at least 1 (e.g., 2, 3, 4, 5, 6, 7, 8, 9, 10, 20, 30, 40, 50, 60, 70, 80, 90, or 100) transgenes. A transgene may encode any gene (e.g., a protein-encoding gene, a gene-targeting nucleic acid, and/or a therapeutic gene). Non-limiting examples of genes include wild-type genes that are mutated in a genetic disease. In certain embodiments, the genes are transcription factors. In certain embodiments, the transgene encodes OCT4, SOX2, KLF4, or homologs or variants (e.g., functional variants) thereof, alone or in combination. In certain embodiments, an engineered nucleic acid encodes c-Myc. In certain embodiments, an engineered nucleic acid does not encode c-Myc. In certain embodiments, an engineered nucleic acid does not encode a functional c-Myc because it lacks a c-Myc sequence. Assays to determine transcription factor (e.g., OCT4, SOX2, KLF4, c-Myc or any combination thereof) activity are known in the art and include cell-based transcription assays and in vitro transcription assays. Transcription factor expression may also be determined using other methods including enzyme-linked immunosorbent assays (ELISAs), western blots, and quantification of RNA (e.g., using reverse transcription polymerase chain reaction).

Non-limiting examples of genes include Tet1, Tet2, Nanog, SIRT1, SIRT2, SIRT3, SIRT4, SIRT5, SIRT6, SIRT7, FGF21, GDF15, GDF11, NF-kb, PCSK9, mTERT, HAS2, PDE4By358c, sIGF1r-FC, sIGF2r-FC, Fat-1, mTOR, Klotho, TFEB, Grin2b, DNMT1, AMPK, NRF2, NEU1, NGF, Bcat-1, FoxP2, ZAG, Adiponectin, and TFAM.

In some embodiments, the transgene encodes a gene-targeting nucleic acid. In some embodiments, the gene-targeting nucleic acid is complementary to the promoter and/or enhancer region of the endogenous locus of a gene. In some embodiments, the gene-targeting nucleic acid is complementary to the protein-coding region of a gene. In some embodiments, the gene-targeting nucleic acid is complementary to a RNA (e.g., mRNA). In some embodiments, the RNA (e.g., mRNA) encodes a protein.

In some embodiments, the gene-targeting nucleic acid is a small interfering RNA (siRNA), small hairpin RNA or short hairpin RNA (shRNA), microRNA (miRNA), guide RNA (gRNA), or an antisense RNA (asRNA). In some embodiments, the siRNA, shRNA, miRNA, or asRNA is capable of decrease expression of the target gene by at least 10%, 20%, 30%, 40%, 50%, 60%, 70%, 80%, 90%, or 100%.

In some embodiments, the guide RNA is a CRISPR guide RNA and can guide a Cas9 nuclease to an endogenous location in the genome. The guide RNA and CRISPR system may be used to knockout expression of a gene or activate expression of a gene. When used with a donor template, the guide RNA and CRISPR system may also be used to knockin an allele or gene of interest. Methods of using CRISPR systems and considerations for guide RNA design are known in the art. Numerous web-based tools are available for designing guide RNAs. For example, a non-limiting example available from MIT (e.g., crispr.mit.edu/) and from the Broad Institute (portals.broadinstitute.org/gpp/public/analysis-tools/sgrna-design). See also, e.g., Hsu et al., Cell. 2014 Jun. 5; 157(6):1262-78; Sander et al., Nat Biotechnol. 2014 April; 32(4):347-55; Doench et al., Nat Biotechnol. 2016 February; 34(2):184-191.

In some embodiments the transgene encodes Cas9 and/or a nuclease-deficient Cas9 that is fused to a transcription activation complex (e.g., comprising VP64, P65, Rta, and/or MPH).

In general, a CRISPR-activating system comprises an enzymatically dead Cas9 nuclease (or nuclease-deficient Cas9 (dCas9)) fused to a transcription activation complex (e.g., comprising VP64, P65, Rta, and/or MPH). Non-limiting examples of sequences encoding VP64, P65, Rta, and/or MPH are provided below. A VP64, P65, Rta, or MPH may ccomprisecomprise a sequence that is at least 70% (e.g., 75%, 80%, 85%, 90%, 95%, 98%, 99%, or 100%) identical to any of the VP64, P65, Rta, and/or MPH sequences described herein. This Cas9 fusion protein may be referred to as a CRISPR activator. A guide RNA targeting the promoter and/or enhancer region of a gene of interest is used in a CRISPR-activating system to target the dCas9-transcription activation complex and drive expression of the endogenous gene.

In some embodiments, administration of an inducible nucleic acid (e.g., an engineered nucleic acid, including an expression vector) comprising a TRE promoter operably linked to a transgene encoding Cas9 and/or a guide RNA targeting an endogenous gene of interest results in decrease expression of the target gene by at least 10%, 20%, 30%, 40%, 50%, 60%, 70%, 80%, 90%, or 100%. In some embodiments, the Cas9 and the guide RNA are located on two separate expression vectors. In some embodiments, administration of an inducible nucleic acid (e.g., an engineered nucleic acid, including an expression vector) comprising a TRE promoter operably linked to a transgene encoding dCas9 fused to an activator complex and/or a guide RNA targeting a promoter and/or enhancer region of an endogenous gene of interest results increasing expression of the operably linked gene by 10%, 20%, 30%, 40%, 50%, 60%, 70%, 80%, 90%, 100%, 150%, 200%, 300%, 400%, 500%, 600%, 700%, 800%, 900%, or 1000%.

The transgenes of the present disclosure (e.g., transgene encoding a protein, a therapeutic sequence, a gene-targeting nucleic acid, a nucleic acid encoding OCT4, SOX2, KLF4, c-Myc, transcription factors, or homologs or variants thereof, including mammalian OCT4, mammalian SOX2, and mammalian KLF4) may be encoded by a single nucleic acid, or a single nucleic acid may encode two or more transgenes (e.g., each operably linked to a different promoter, or all operably linked to the same promoter). For example, in certain embodiments, a nucleic acid may encode OCT4; SOX2; c-Myc; KLF4; OCT4 and SOX2; OCT4 and KLF4; SOX2 and KLF4; or OCT4, SOX2, and KLF4, OCT4, c-Myc, and SOX2; OCT4, c-Myc, and KLF4; SOX2, c-Myc, and KLF4; or OCT4, SOX2, c-Myc, and KLF4, in any order.

In certain embodiments, a nucleic acid (e.g., an engineered nucleic acid, including an expression vector) encodes at least one (e.g., 2, 3, 4, 5, 6, 7, 8, 9, 10, 11, 12, 13, 14, 15, 16, 17, 18, 19, 20, 30, 40, 50, 60, 70, 80, 90, or 100) transgene.

In certain embodiments, a transgene encodes a protein. In certain embodiments, the protein is a human protein. In certain embodiments, the protein is a non-human protein (for example, mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds, such as chickens, ducks, geese, and/or turkeys). If two or more of the transgenes are on one vector, they may be in any order. Ther words “first,” “second,” and “third” are not meant to imply an order of the genes on the vector.

In certain embodiments, a transgene encodes a therapeutic sequence. Therapeutic sequences may also be referred to as genes that suitable for gene therapy in the art. Non-limiting examples of therapeutic proteins include antibodies, enzymes, kinases, hormones, growth factors, cytokines, plasma proteins, fusion proteins, membrane-lytic proteins and coagulation factors. In some embodiments, a therapeutic protein is an inflammatory agent. In some embodiments, a therapeutic protein is an anti-inflammatory agent. In some embodiments, a therapeutic protein is an immunomodulatory agent. In some embodiments, a therapeutic protein is an anti-cancer agent. In some embodiments, a therapeutic protein is a metabolic agent. In some embodiments, a therapeutic protein is an antiviral/virocidal agent. In some embodiments, a therapeutic protein is an antibacterial/bacteriocidal agent.

The transgenes described herein (e.g., a therapeutic sequence, a gene-targeting nucleic acid) may comprise one or more amino acid substitutions. Variants can be prepared according to methods for altering polypeptide sequences known to one of ordinary skill in the art such as those found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

In certain embodiments, a nucleic acid (e.g., an engineered nucleic acid, including an expression vector) comprising a transgene (e.g., a protein-encoding gene, a gene-targeting nucleic acid, and/or a therapeutic gene) is present on a viral vector (e.g., AAV vector). An AAV vector, as used herein, generally comprises ITRs flanking an expression cassette (e.g., a nucleic acid comprising a TRE promoter operably linked to a transgene (e.g., a therapeutic sequence, a gene-targeting nucleic acid and/or a protein-encoding sequence)).

In certain embodiments, a nucleic acid (e.g., an engineered nucleic acid, including an expression vector) comprising a transgene (e.g., a protein-encoding gene, a gene-targeting nucleic acid, and/or a therapeutic gene) is present on a non-viral vector (e.g., a plasmid for transient transfection).

In certain embodiments, the number of base pairs between two ITRs in an AAV vector of the present disclosure is less than 5 kilobases (kb) (e.g., less than 4.9 kb, less than 4.8 kb, less than 4.7 kb, less than 4.6 kb, less than 4.5 kb, less than 4.4 kb, less than 4.3 kb, less than 4.2 kb, less than 4.1 kb, less than 4 kb, less than 3.5 kb, less than 3 kb, less than 2.5 kb, less than 2 kb, less than 1.5 kb, less than 1 kb, or less than 0.5 kb). In certain embodiments, an AAV vector with a distance of less than 4.7 kb between two ITRs is capable of being packaged into virus at a titer of at least 0.5×10\hat{ }10 particle forming units per ml (pfu/ml), at least 1×10\hat{ }10 pfu/ml, at least 5×10\hat{ }10 pfu/ml, at least 1×10\hat{ }11 pfu/ml, at least 5×10\hat{ }11 pfu/ml, at least 1×10\hat{ }12 pfu/ml, at least 2×10\hat{ }12 pfu/ml, at least 3×10\hat{ }12 pfu/ml, at least 4×10\hat{ }12 pfu/ml, at least 5×10\hat{ }12 pfu/ml, at least 6×10\hat{ }12 pfu/ml, at least 7×10\hat{ }12 pfu/ml, at least 8×10\hat{ }12 pfu/ml, at least 9×10\hat{ }12 pfu/ml, or at least 1×10\hat{ }13 pfu/ml.

In certain embodiments, a nucleic acid (e.g., an engineered nucleic acid, including an expression vector) of the present disclosure is at least 1 kilobase (kb) (e.g., at least 1 kb, 2 kb, 3 kb, 4 kb, 5 kb, 6 kb, 7 kb, 8 kb, 9 kb, 10 kb, 50 kb, or 100 kb). In certain embodiments, a nucleic acid (e.g., an engineered nucleic acid, including an expression vector) of the present disclosure is less than 10 kb (e.g., less than 9 kb, less 8 kb, less than 7 kb, less than 6 kb, less than 5 kb, less than 4 kb, less than 3 kb, less than 2 kb, or less than 1 kb).

Without being bound by a particular theory, a nucleic acid (e.g., an engineered nucleic acid, including an expression vector) (e.g., an AAV vector) that encodes multiple transgenes (e.g., protein-encoding sequences, a gene-targeting nucleic acid, and/or therapeutic sequences) under one promoter results in more efficient transduction of all transgenes in vivo compared to separate nucleic acids encoding one or two of the transgenes. In certain embodiments, the infection efficiency of a recombinant virus (e.g., lentivirus, adenovirus, alphavirus, vaccinia virus, retrovirus, herpes virus, or AAV) harboring a transgene vector of the present disclosure in cells (e.g., eukaryotic cells or prokaryotic cells) is at least 20% (e.g., at least 30%, at least 40%, at least 50%, at least 60%, at least 70%, at least 80%, or at least 90%, or 100%).

### Recombinant Viruses

Aspects of the present disclosure provide recombinant viruses (e.g., lentiviruses, adenoviruses, herpes viruses, alphaviruses, vaccinia viruses, retroviruses, or AAVs). The recombinant viruses (e.g., lentivirus, adenovirus, alphavirus, vaccinia virus, retrovirus, herpes virus, or AAV) may harbor a nucleic acid (e.g., an engineered nucleic acid, including an expression vector) encoding a mutant rtTA, an inducible nucleic acid (e.g., an engineered nucleic acid, including an expression vector) comprising a transgene (e.g., a therapeutic sequence, a gene-targeting nucleic acid, and/or a protein encoding sequence), or a combination thereof.

In certain embodiments, recombinant virus is a recombinant AAV. In some embodiments, a recombinant AAV has tissue-specific targeting capabilities, such that a transgene of the AAV will be delivered specifically to one or more predetermined tissue(s). Generally, the AAV capsid is a relevant factor in determining the tissue-specific targeting capabilities of an AAV. An AAV capsid may comprise an amino acid sequence derived from AAV1, AAV2, AAV3, AAV4, AAV5, AAV6, AAV7, AAV8, AAV9, AAV10, AAV11, and variants thereof. Non-limiting examples of the tissue-specificity of AAV serotypes are provided in Table 1. An “x” indicates that the indicated AAV serotype is capable of delivering a transgene to a specific tissue.

TABLE 1
Non-limiting examples of AAV serotypes and their utility in specific tissues.

| | Relevant Tissue | | | | | | | | |
|---|---|---|---|---|---|---|---|---|---|
| | | | | | | Central | | | Immune |
| | | | | | | Nervous | | | System |
| | | | Muscle | | Central | System | | | (T-cells, |
| | | | (e.g., | | Nervous | (Blood- | | | B-cells and |
| | | | Skeletal | | System | brain | | | Dendritic |
| AAV serotype | Liver | Heart | Muscle) | Eye | (CNS) | barrier) | Pancreas | Lung | Cells) |
| AAV1 | | x | x | | x | | | | |
| AAV2 | x | | x | x | x | | | | |
| AAV3 | x | | x | x | | | | x | |
| AAV4 | | | x | x | x | | | | |
| AAV5 | | | | x | x | | x | x | |
| AAV6 (e.g., | | x | x | | | | | x | x |
| AAV6.2) | | | | | | | | | |
| AAV7 | x | | x | | | | | | |
| AAV8 | x | | x | | x | | x | | |
| AAV9 | x | x | x | x | x | x | x | x | |
| AAV10 (e.g., | x | x | x | x | x | x | x | x | |
| AAVrh10) | | | | | | | | | |
| AAVDJ | x | | x | | x | | | | |
| AAVPHP.B | | | | | x | x | | | |

Recombinant AAVs comprising a particular capsid protein may be produced using any suitable method. See, e.g., U.S. Patent Application Publication, US 2003/0138772, which is incorporated herein by reference. AAV capsid protein sequences also known in the art. See, e.g., Published PCT Application, WO 2010/138263, which is incorporated herein by reference. Generally, recombinant AAV is produced in a host cell with the following components: (1) a nucleic acid sequence encoding an AAV capsid protein or a fragment thereof, (2) a nucleic acid encoding a functional rep gene, (3) a recombinant AAV vector comprising AAV inverted terminal repeats flanking a transgene (e.g., a sequence encoding a protein, a gene targeting nucleic acid, and/or a therapeutic sequence), and (4) helper functions that allow for packaging of the recombinant AAV vector into AAV capsid proteins. In certain embodiments, the helper functions are introduced via a helper vector that is known in the art.

In some instances, a suitable host cell line (e.g., BEWK293T cells) may be used for producing a recombinant AAV disclosed herein following routine practice. One or more expression vectors encoding one or more of the components described above may be introduced into a host cell by exogenous nucleic acids, which can be cultured under suitable conditions allowing for production of AAV particles. When needed, a helper vector can be used to facilitate replication, to facilitate assembly of the AAV particles, or any combination thereof. In certain embodiments, the recombinant AAV vector is present on a separate nucleic acid from the other components (e.g., a nucleic acid sequence encoding an AAV capsid protein or a fragment thereof, a nucleic acid encoding a functional rep gene, and helper functions that allow for packaging of the recombinant AAV vector into AAV capsid proteins. In certain embodiments, a host cell may stably express one or more components needed to produce AAV virus. In that case, the remaining components may be introduced into the host cell. The supernatant of the cell culture may be collected, and the viral particles contained therein can be collected via routine methodology.

The compositions of the disclosure may comprise any of the recombinant viruses (e.g., lentivirus, adenovirus, alphavirus, vaccinia virus, retrovirus, herpes virus, or AAV) described herein alone, or in combination with one or more other recombinant viruses (e.g., a second AAV encoding having one or more different transgenes). In some embodiments, a composition comprises 1, 2, 3, 4, 5, 6, 7, 8, 9, 10, or more different viruses (e.g., lentivirus, adenovirus, alphavirus, vaccinia virus, retrovirus, herpes virus, or AAV) each having one or more different transgenes.

In some embodiments, a composition further comprises a pharmaceutically acceptable carrier. Suitable carriers may be readily selected by one of skill in the art in view of the indication for which the recombinant virus (e.g., lentivirus, adenovirus, alphavirus, vaccinia virus, retrovirus, herpes virus, or AAV) is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the recombinant virus (e.g., lentivirus, adenovirus, alphavirus, vaccinia virus, retrovirus, herpes virus, or AAV) and carrier(s), other pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The recombinant viruses (e.g., lentivirus, adenovirus, alphavirus, vaccinia virus, retrovirus, herpes virus, or AAV) are administered in sufficient amounts to transfect the cells of a desired tissue (e.g., ocular tissue, such as corneal tissue) and to provide sufficient levels of gene transfer and expression without undue adverse effects. Examples of pharmaceutically acceptable routes of administration include, but are not limited to, direct delivery to the selected organ (e.g., intrastromal delivery to the eye), oral, inhalation (including intranasal and intratracheal delivery), intraocular, intravenous, intramuscular, subcutaneous, intradermal, intratumoral, and other parental routes of administration. Routes of administration may be combined, if desired.

The dose of recombinant virus (e.g., lentivirus, adenovirus, alphavirus, vaccinia virus, retrovirus, herpes virus, or AAV) virions required to achieve a particular therapeutic effect, e.g., the units of dose in genome copies/per kilogram of body weight (GC/kg), will vary based on several factors including, but not limited to: the route of recombinant virus (e.g., lentivirus, adenovirus, alphavirus, vaccinia virus, retrovirus, herpes virus, or AAV) virion administration, the level of gene or RNA expression required to achieve a therapeutic effect, the specific disease or disorder being treated, and the stability of the gene or RNA product. One of skill in the art can readily determine a recombinant virus (e.g., lentivirus, adenovirus, alphavirus, vaccinia virus, retrovirus, herpes virus, or AAV virion) dose range to treat a patient having a particular disease or disorder based on the aforementioned factors, as well as other factors.

An effective amount of a recombinant virus (e.g., lentivirus, adenovirus, alphavirus, vaccinia virus, retrovirus, herpes virus, or AAV) is an amount sufficient to target infect an animal, target a desired tissue. In some embodiments, an effective amount of an recombinant virus (e.g., lentivirus, adenovirus, alphavirus, vaccinia virus, retrovirus, herpes virus, or AAV) is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the recombinant virus (e.g., lentivirus, adenovirus, alphavirus, vaccinia virus, retrovirus, herpes virus, or AAV) is generally in the range of from about 1 ml to about 100 ml of solution containing from about 10⁹ to 10¹⁶ genome copies. In some cases, a dosage between about 10¹¹ to 10¹³ recombinant virus (e.g., lentivirus, adenovirus, alphavirus, vaccinia virus, retrovirus, herpes virus, or AAV) genome copies is appropriate. In certain embodiments, 10¹⁰ or 10¹¹ recombinant virus (e.g., lentivirus, adenovirus, alphavirus, vaccinia virus, retrovirus, herpes virus, or AAV) genome copies is effective to target ocular tissue (e.g., retinal tissue). In some cases, stable transgenic animals are produced by multiple doses of a recombinant virus (e.g., lentivirus, adenovirus, alphavirus, vaccinia virus, retrovirus, herpes virus, or AAV).

In some embodiments, a dose of recombinant virus (e.g., lentivirus, adenovirus, alphavirus, vaccinia virus, retrovirus, herpes virus, or AAV) is administered to a subject no more than once per calendar day (e.g., a 24-hour period). In some embodiments, a dose of recombinant virus (e.g., lentivirus, adenovirus, alphavirus, vaccinia virus, retrovirus, herpes virus, or AAV) is administered to a subject no more than once per 2, 3, 4, 5, 6, or 7 calendar days. In some embodiments, a dose of recombinant virus (e.g., lentivirus, adenovirus, alphavirus, vaccinia virus, retrovirus, herpes virus, or AAV) is administered to a subject no more than once per calendar week (e.g., 7 calendar days). In some embodiments, a dose of recombinant virus (e.g., lentivirus, adenovirus, alphavirus, vaccinia virus, retrovirus, herpes virus, or AAV) is administered to a subject no more than bi-weekly (e.g., once in a two calendar week period). In some embodiments, a dose of recombinant virus (e.g., lentivirus, adenovirus, alphavirus, vaccinia virus, retrovirus, herpes virus, or AAV) is administered to a subject no more than once per calendar month (e.g., once in 30 calendar days). In some embodiments, a dose of recombinant virus (e.g., lentivirus, adenovirus, alphavirus, vaccinia virus, retrovirus, herpes virus, or AAV) is administered to a subject no more than once per six calendar months. In some embodiments, a dose of recombinant virus (e.g., lentivirus, adenovirus, alphavirus, vaccinia virus, retrovirus, herpes virus, or AAV) is administered to a subject no more than once per calendar year (e.g., 365 days or 366 days in a leap year).

In some embodiments, recombinant virus (e.g., lentivirus, adenovirus, alphavirus, vaccinia virus, retrovirus, herpes virus, or AAV) compositions are formulated to reduce aggregation of AAV particles in the composition, particularly where high recombinant virus (e.g., lentivirus, adenovirus, alphavirus, vaccinia virus, retrovirus, herpes virus, or AAV) concentrations are present (e.g., ~10¹³ GC/ml or more). Appropriate methods for reducing aggregation of may be used, including, for example, addition of surfactants, pH adjustment, salt concentration adjustment, etc. (See, e.g., Wright F R, et al., Molecular Therapy (2005) 12, 171-178, the contents of which are incorporated herein by reference.)

In some embodiments, a nucleic acid is delivered non-virally (e.g., not on a viral vector and/or not in a virus). In some embodiments, a nucleic acid (e.g., RNA or DNA) encoding a transgene operably linked to a TRE promoter and/or a mutant rtTA (e.g., rtTA4) is administered in a liposome. In some embodiments, the nucleic acid is RNA (e.g., mRNA). In some embodiments, a nucleic acid (e.g., RNA or DNA) encoding a transgene operably linked to a TRE promoter and/or a mutant rtTA (e.g., rtTA4) is administered in a nanoparticle.

Systems and Recombinant Cells Comprising a Mutant rtTA or a Nucleic Acid (e.g., an Engineered Nucleic Acid, Including an Expression Vector) Thereof

Aspects of the present disclosure also provide systems or cells comprising a mutant rtTA (e.g., rtTA protein) and/or a nucleic acid (e.g., an engineered nucleic acid, including an expression vector) encoding a mutant rtTA thereof. The cells may be eukaryotic or prokaryotic and may be from any tissue (e.g., ear, nose, mouth including gum and roots of teeth, bone, lung, breast, udder, pancreas, stomach, oesophagus, muscle including cardiac muscle, liver, blood vessel, skin including hair, heart, brain, nerve tissue, kidney, testis, prostate, penis, cloaca, fin, ovary, or intestine).

In certain embodiments, a system or cell comprises a nucleic acid (e.g., an engineered nucleic acid, including an expression vector) encoding both a mutant rtTA and a transgene operably linked to an inducible promoter that comprises a Tet-O sequence. In certain embodiments, a system or cell comprises a nucleic acid (e.g., an engineered nucleic acid, including an expression vector) encoding a mutant rtTA and a second nucleic acid (e.g., an engineered nucleic acid, including an expression vector) comprising a transgene operably linked to an inducible promoter that comprises a Tet-O sequence. In certain embodiments, a system or cell comprises multiple (e.g., at least 1, 2, 3, 4, 5, 6, 7, 8, 9, 10, 20, 30, 40, 50, 60, 70, 80, 90, or 100) expression vectors encoding one or more transgenes (e.g., at least 1, 2, 3, 4, 5, 6, 7, 8, 9, 10, 20, 30, 40, 50, 60, 70, 80, 90, or 100 transgenes).

In some embodiments, a nucleic acid encoding a mutant rtTA (e.g., rtTA4) and/or a TRE promoter (e.g., TRE3G, TRE2, and/or P tight) is integrated into the genome of a cell. In some embodiments, a nucleic acid encoding a mutant rtTA (e.g., rtTA4) and/or a TRE promoter (e.g., TRE3G, TRE2, and/or P tight) is integrated into the genome of a subject (e.g., to create a transgenic subject). Any suitable method may be used to integrate a nucleic acid encoding a mutant rtTA (e.g., rtTA4) and/or a TRE promoter. See, e.g., Cho et al., Curr Protoc Cell Biol. 2009 March; CHAPTER: Unit-19.11. In some embodiments, a mutant rtTA and/or TRE promoter is knocked in to a cell or into a subject using CRISPR. See, e.g., Aida et al., Genome Biol. 2015 Apr. 29; 16:87. As a non-limiting example, rtTA may be integrated into the rosa 26 locus in a mouse and/or TRE may be integrated into the Col1a1 locus.

In some embodiments, a recombinant cell is produced ex vivo and administered to a subject in need thereof.

### Compositions (e.g., Pharmaceutical Compositions)

Aspects of the present disclosure provide compositions comprising any of the mutant rtTAs, recombinant cells, nucleic acids (e.g., engineered nucleic acids, expression vectors, plasmid DNA, and/or RNA) encoding a rtTA, inducible vectors encoding a transgene, recombinant viruses encoding a rtTA and/or a transgene, or combinations thereof. Any of the mutant rtTAs, recombinant cells, nucleic acids (e.g., engineered nucleic acids, expression vectors, plasmid DNA, and/or RNA) encoding a rtTA, inducible vectors encoding a transgene, recombinant viruses encoding a rtTA and/or a transgene, or combinations thereof may be formulated into a pharmaceutical composition with a pharmaceutically acceptable excipient. Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, the mutant rtTAs, recombinant cells, nucleic acids (e.g., engineered nucleic acids, expression vectors, plasmid DNA, and/or RNA) encoding a rtTA, inducible vectors encoding a transgene, and/or recombinant viruses described herein are suitably formulated pharmaceutical compositions disclosed herein are delivered directly to target tissue and/or organ in a subject, e.g., direct to eye, ear, nose, mouth including gum and roots of teeth, bone, lung, breast, udder, pancreas, stomach, oesophagus, muscle including cardiac muscle, liver, blood vessel, skin including hair, heart, brain, nerve tissue, kidney, testis, prostate, penis, cloaca, fin, ovary, or intestine.

However, in certain circumstances it may be desirable to separately or in addition deliver the recombinant viruses, recombinant cells, nucleic acids (e.g., engineered nucleic acids), and/or mutant rtTA (e.g., rtTA4) via another route, e.g., intravenously, intradermally, intraarterially, intralesionally, intratumorally, intracranially, intraarticularly, intraprostaticaly, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, systemically, injection, infusion, continuous infusion, localized perfusion bathing target cells directly, via a catheter, in creams, in lipid compositions (e.g., liposomes).

In some embodiments, the administration modalities as described in U.S. Pat. Nos. 5,543,158; 5,641,515 and 5,399,363 (each specifically incorporated herein by reference in its entirety) may be used to deliver recombinant virus (e.g., lentivirus, adenovirus, alphavirus, vaccinia virus, retrovirus, herpes virus, or AAV). In some embodiments, a preferred mode of administration is by intrastromal injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a suitable sterile aqueous medium may be employed. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active recombinant virus (e.g., lentivirus, adenovirus, alphavirus, vaccinia virus, retrovirus, herpes virus, or AAV) in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The recombinant virus (e.g., lentivirus, adenovirus, alphavirus, vaccinia virus, retrovirus, herpes virus, or AAV) compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

A carrier includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions.

Delivery vehicles such as liposomes, nanocapsules, microparticles, microspheres, lipid particles, vesicles, and the like, may be used for the introduction of compositions of the present disclosure into suitable host cells. As a non-limiting example, the recombinant virus (e.g., lentivirus, adenovirus, alphavirus, vaccinia virus, retrovirus, herpes virus, or AAV) vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids (e.g., engineered nucleic acids, including expression vectors) or the recombinant virus (e.g., lentivirus, adenovirus, alphavirus, vaccinia virus, retrovirus, herpes virus, or AAV) constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587; each of which is incorporated herein by reference).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MHLVs results in the formation of small unilamellar vesicles (SUVs) with diameters in the range of 200 to 500 .ANG., containing an aqueous solution in the core.

Alternatively, nanocapsule formulations of the recombinant virus (e.g., lentivirus, adenovirus, alphavirus, vaccinia virus, retrovirus, herpes virus, or AAV) may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

In some embodiments, the instant disclosure relates to a kit comprising a nucleic acid (e.g., an engineered nucleic acid, including an expression vector) encoding mutant rtTA (e.g., rtTA4), which may be useful, for example for producing a recombinant virus (e.g., lentivirus, adenovirus, alphavirus, vaccinia virus, retrovirus, herpes virus, or AAV). The kit may comprise a container housing the nucleic acid (e.g., an engineered nucleic acid, including an expression vector) encoding mutant rtTA (e.g., rtTA4). The kit may further comprise a second container housing a nucleic acid (e.g., an engineered nucleic acid, including an expression vector) encoding a transgene (e.g., a gene associated a disease, such as retinal disease). In certain embodiments, the transgene is a sequence encoding a protein, a gene-targeting nucleic acid, and/or a therapeutic sequence. In some embodiments, the kit further comprises instructions for producing recombinant virus (e.g., lentivirus, adenovirus, alphavirus, vaccinia virus, retrovirus, herpes virus, or AAV).

In some embodiments, the instant disclosure relates to a kit comprising a container housing any of the engineered nucleic acids (e.g., expression vectors) or recombinant viruses described herein. For example, the kit may comprise a nucleic acid (e.g., an engineered nucleic acid, including an expression vector) or recombinant virus encoding an inducing agent. In some embodiments, a nucleic acid (e.g., an engineered nucleic acid, including an expression vector) a mutant rtTA4 comprises a sequence that is at least 70% identical to SEQ ID NO: 17. In some embodiments, the nucleic acid (e.g., an engineered nucleic acid, including an expression vector) encoding an inducing agent consists of a sequence that is at least 70% identical to SEQ ID NO: 17. In some embodiments, a nucleic acid (e.g., an engineered nucleic acid, including an expression vector) a mutant rtTA4 comprises a sequence that is at least 70% identical to desmin-rtTA4 (SEQ ID NO: 30). In some embodiments, the nucleic acid (e.g., an engineered nucleic acid, including an expression vector) encoding an inducing agent consists of a sequence that is at least 70% identical to desmin-rtTA4 (SEQ ID NO: 30). The kit may further comprise a nucleic acid (e.g., an engineered nucleic acid, including an expression vector) or recombinant virus encoding any transgene (e.g., therapeutic sequence, a gene-targeting nucleic acid, and/or sequence encoding a protein) operably linked to a promoter comprising a Tet-O (e.g., a TRE promoter). A non-limiting example of a vector encoding a transgene (e.g., multiple transgenes) is provided in SEQ ID NO: 16. In some embodiments, the nucleic acid (e.g., an engineered nucleic acid, including an expression vector) encoding a transgene comprises SEQ ID NO: 16.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to an animal, such as a syringe, topical application devices, or iv needle tubing and bag, particularly in the case of the kits for producing specific somatic animal models.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

### Therapeutic Applications

Any of the compositions (e.g., pharmaceutical compositions) comprising a mutant rtTA (e.g., rtTA4), a nucleic acid (e.g., an engineered nucleic acid, including an expression vector) encoding a mutant rtTA, an inducible nucleic acid (e.g., an engineered nucleic acid, including an expression vector) encoding a transgene, a recombinant virus encoding a mutant rtTA or a transgene that is operably linked to an inducible promoter encoding described herein may be used to regulate (e.g., inhibit or induce) cellular reprogramming, tissue repair, tissue regeneration, treating a disease, organ regeneration, reversing aging, or any combination thereof. The compositions may be useful in regulating cellular reprogramming, tissue repair, tissue survival, tissue regeneration, tissue growth, tissue function, organ regeneration, organ survival, organ function, or any combination thereof. Regulating may comprise inducing cellular reprogramming, reversing aging, improving tissue function, improving organ function, tissue repair, tissue survival, tissue regeneration, tissue growth, angiogenesis, scar formation, the appearance of aging, organ regeneration, organ survival, altering the taste and quality of agricultural products derived from animals, treating a disease, or any combination thereof, in vivo or in vitro may be administered to a cell, tissue, or organ that is in vivo (e.g., part of a subject), or may be administered to a cell, tissue, or organ ex vivo. As used herein, regulating may refer to any type of modulation, including inducing or promoting, inhibiting, and/or stopping. Angiogenesis refers to growth of new blood vessels.

In some instances, a viral vector (e.g., lentivirus, adenovirus, alphavirus, vaccinia virus, retrovirus, herpes virus, or AAV vector) is administered in a recombinant virus (e.g., lentivirus, adenovirus, alphavirus, vaccinia virus, retrovirus, herpes virus, or AAV).

Without being bound by a particular theory, transient expression of one or more transgenes (e.g., OCT4, SOX2, KLF4, any transcription factor, any protein-encoding sequence, any gene-targeting nucleic acid, and/or any therapeutic sequence) may result in partial reprogramming of a cell. For example, partial reprogramming may induce a fully differentiated cell to become multipotent. In some instances, prolonged expression (e.g., continued expression for at least 1 day, at least 5 days, at least 1 week, or at least 1 month) of one or more transcription factors (e.g., any transcription factor, including OCT4, SOX2, KLF4, and/or c-MYC), results in full reprogramming of a cell. For example, a cell may be fully reprogrammed into a pluripotent cell (e.g., induced pluripotent cell).

To practice this embodiment, an effective amount of mutant rtTA (e.g., rtTA4 may be administered to a cell, tissue or a subject along with a nucleic acid (e.g., an engineered nucleic acid, including an expression vector) comprising a transgene to be expressed (a protein-encoding sequence, a gene-targeting nucleic acid, and/or a therapeutic sequence) operably linked to a TRE promoter (e.g., a TRE2, a P tight, or a TRE3G promoter). In certain embodiments, mutant rtTA (e.g., rtTA4) is administered as a protein. In certain embodiments, mutant rtTA (e.g., rtTA4) is administered in a nucleic acid (e.g., an engineered nucleic acid, including an expression vector)

In some embodiments, a nucleic acid (e.g., engineered nucleic acid) encoding mutant rtTA4 and/or a transgene operably linked to a TRE promoter (e.g., TRE3G, a P tight, or a TRE2 promoter) is not a viral vector. For example, the nucleic acid may be a plasmid (e.g., plasmid DNA) or RNA (e.g., mRNA). As a non-limiting example, the engineered nucleic acids (e.g., RNA, including mRNA, or DNA) of the present disclosure may be formulated in a nanoparticle for delivery. See, e. g., Dong et al., Nano Lett. 2016 Feb. 10; 16(2):842-8. In some embodiments, the nanoparticle comprises acetylated galactose. See, e.g., Lozano-Torres et al., J Am Chem Soc. 2017 Jul. 5; 139(26):8808-8811. In some embodiments, an engineered nucleic acids (e.g., RNA, including mRNA, or DNA) is electroporated or transfected into a cell. In certain embodiments, the engineered nucleic acids are delivered as a naked nucleic acid (e.g., naked DNA or naked RNA). In some embodiments, a naked nucleic acid is plasmid DNA. In some embodiments, a nucleic acid (e.g., engineered nucleic acid) is administered in a liposome.

In some embodiments, a nucleic acid (e.g., engineered nucleic acid) encoding mutant rtTA4 and/or encoding a transgene operably linked to a TRE promoter (e.g., e.g., TRE3G, a P tight, or a TRE2 promoter) is a viral vector. Non-limiting examples of viral vectors include lentivirus vector, adenovirus vector, alphavirus vector, vaccinia virus vector, herpes virus vector, adenovirus (AAV) vector.

In certain embodiments, a nucleic acid (e.g., engineered nucleic acid encoding a mutant rtTA (e.g., rtTA4)) is administered in a virus (e.g., lentivirus, adenovirus, alphavirus, vaccinia virus, retrovirus, herpes virus, or AAV). In certain embodiments, the nucleic acid (e.g., an engineered nucleic acid, including an expression vector) comprising a transgene operably linked to a TRE promoter (e.g., TRE3G, a P tight, or a TRE2 promoter) is administered in a virus (e.g., lentivirus, adenovirus, alphavirus, vaccinia virus, retrovirus, herpes virus, or AAV)

A suitable amount of tetracycline (e.g., doxycycline) may be added to drive expression from the TRE promoter (e.g. TRE3G, P tight, or a TRE2 promoter). The suitable amount of tetracycline to be added may be determined by one of ordinary skill in the art and may depend on factors, including the type of pharmaceutical excipient (if any), the type of cell, the type of tissue, or any characteristic of a subject (e.g., weight, medical history, genetics, etc.).

In some embodiments, tetracycline is administered intravenously, intradermally, intraarterially, intralesionally, intratumorally, intracranially, intraarticularly, intraprostaticaly, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, systemically, injection, infusion, continuous infusion, localized perfusion bathing target cells directly, via a catheter, in creams, or in lipid compositions.

In some embodiments, a recombinant virus and/or expression vector encoding a mutant rtTA and/or a transgene operably linked to a TRE promoter is administered systemically. In some embodiments, the, a recombinant virus and/or expression vector encoding a mutant rtTA and/or a transgene operably linked to a TRE promoter is administered locally (e.g., directly to a tissue or organ of interest, including eye, ear, nose, mouth including gum and roots of teeth, bone, lung, breast, udder, pancreas, stomach, oesophagus, muscle including cardiac muscle, liver, blood vessel, skin including hair, heart, brain, nerve tissue, kidney, testis, prostate, penis, cloaca, fin, ovary, or intestine).

In some embodiments, the virus and/or expression vector is administered with tetracycline (e.g., doxycycline). In some embodiments, the virus and/or expression vector comprising a TRE promoter is administered separately from tetracycline (e.g., doxycycline). For example, any of the viruses and/or expression vectors comprising a TRE promoter described herein may be administered systemically and the tetracycline may be administered locally (e.g., to an organ or tissue of interest). In some embodiments, any of the viruses and/or expression vectors comprising a TRE promoter described herein may be administered locally (e.g., to directly to a tissue or organ of interest, including eye, ear, nose, mouth including gum and roots of teeth, bone, lung, breast, udder, pancreas, stomach, oesophagus, muscle including cardiac muscle, liver, blood vessel, skin including hair, heart, brain, nerve tissue, kidney, testis, prostate, penis, cloaca, fin, ovary, or intestine) and the tetracycline and/or mutant rtTA (e.g., mutant rtTA protein or nucleic acid encoding a mutant rtTA) may be administered systemically. As a non-limiting example, a virus and/or expression vector comprising a TRE promoter is administered directly (e.g., injected) into the eye of a subject and the tetracycline (e.g., doxycycline) and/or mutant rtTA (e.g., mutant rtTA protein or nucleic acid encoding a mutant rtTA) is administered systemically (e.g., orally as a pill). In some embodiments the nucleic acid (e.g., engineered nucleic acid) comprising a TRE promoter is administered to a subject in the same route as the mutant rtTA (e.g., mutant rtTA protein or nucleic acid encoding a mutant rtTA). In some embodiments, the nucleic acid (e.g., engineered nucleic acid) comprising a TRE promoter is administered to a subject in a different route as the mutant rtTA (e.g., mutant rtTA protein or nucleic acid encoding a mutant rtTA).

In certain embodiments of the present disclosure, the method further comprises withdrawing tetracycline (e.g., doxycycline) from a cell, tissue, or subject after administration of tetracycline, which may be useful in stopping expression of a transgene. Tetracycline may be withdrawn at least 1 hour, at least 2 hours, at least 3 hours, at least 4 hours, at least 5 hours, at least 10 hours, at least 24 hours, at 2 days, at least 3 days, at least 4 days, at least 5 days, at least 6 days, at least 1 week, at least 2 weeks, at least 3 weeks, at least 1 month, or at least 1 year after tetracycline administration. In certain embodiments, withdrawal of tetracycline results in a detectable decrease in transgene expression (e.g., at least 5%, at least 10%, at least 20%, at least 30%, at least 40%, at least 50%, at least 60%, at least 70%, at least 80%, at least 90%, or 100% decrease). For example, decrease in transgene expression may be detectable at least 1 hour, at least 2 hours, at least 3 hours, at least 4 hours, at least 5 hours, at least 10 hours, at least 24 hours, at 2 days, at least 3 days, at least 4 days, at least 5 days, at least 6 days, at least 1 week, at least 2 weeks, at least 3 weeks, at least 1 month, or at least 1 year after tetracycline withdrawal.

Without being bound by a particular theory, the four mutations in rtTA4 (mutations at positions corresponding to G12, F67, R171, or G72 in SEQ ID NO: 11, for example G12S, G72V or G72P, F67S, and R171K) result in lower leakiness as compared to rtTA3 (SEQ ID NO: 11) because the mutations decrease rtTA4's binding affinity for the promoter in the absence of tetracycline. In some embodiments, the amount of transgene expression with rtTA4 (e.g., SEQ ID NO: 13) is at least 10%, 20%, 30%, 40%, 50%, 60%, 70%, 80%, 90%, or 100% less than the amount of transgene expression detected with rtTA3 (e.g., SEQ ID NO: 11) in the absence of tetracycline (e.g., doxycycline). Without being bound by a particular theory, the four mutations in rtTA4 (mutations at positions corresponding to G12, F67, R171, or G72 in SEQ ID NO: 11, for example G12S, G72V or G72P, F67S, and R171K) result in a greater sensitivity to tetracycline withdrawal as compared to rtTA3 (SEQ ID NO: 11) because the mutations decrease rtTA's binding affinity for tetracycline. In some embodiments, the amount of transgene expression with rtTA4 (e.g., SEQ ID NO: 13) decreases at least 2 times, 3 times, 4 times, 5 times, 6 times, 7 times, 8 times, 9 times, 10 times, 20 times, 30 times, 40 times, 50 times, 60 times, 70 times, 80 times, 90 times, or 100 times faster, when tetracycline (e.g., doxycycline) is withdrawn for a given amount of time compared to the amount of transgene expression with rtTA3 (e.g., SEQ ID NO: 11).

Administration of a nucleic acid (e.g., an engineered nucleic acid, including an expression vector) encoding a mutant rtTA (e.g., rtTA4), a nucleic acid (e.g., an engineered nucleic acid, including an expression vector) comprising a TRE promoter (e.g., TRE3G, TRE2, or a P tight promoter) operably linked to a transgene (e.g., a therapeutic sequence, a gene-targeting nucleic acid, and/or a protein-encoding sequence), and tetracycline increases expression of the transgene by at least 10%, at least 20%, at least 30%, at least 40%, at least 50%, at least 60%, at least 70%, at least 80%, at least 90%, at least 100%, at least 200%, at least 300%, at least 400%, at least 500%, at least 500%, at least 600%, at least 700%, at least 800%, at least 900%, or at least 1,000% in a cell. Gene expression may be determined by routine methods including enzyme-linked immunosorbent assays (ELISAs), western blots, and quantification of RNA (e.g., reverse transcription polymerase chain reaction).

A pharmaceutical composition described herein may be administered to a subject in need thereof. Non-limiting examples of subjects include any animal (e.g., mammals, including humans). A subject may be suspected of having, be at risk for or have a condition. For example, the condition may be an injury or a disease and the condition may affect any tissue (e.g., ear, nose, mouth including gum and roots of teeth, bone, lung, breast, udder, pancreas, stomach, oesophagus, muscle including cardiac muscle, liver, blood vessel, skin including hair, heart, brain, nerve tissue, kidney, testis, prostate, penis, cloaca, fin, ovary, or intestine). Non-limiting examples of conditions, diseases, and disorders include acute injuries, neurodegenerative disease, chronic diseases, proliferative diseases, cardiovascular diseases, genetic diseases, inflammatory diseases, autoimmunue diseases, neurological diseases, hematological diseases, painful conditions, psychiatric disorders, metabolic disorders, cancers, aging, age-related diseases, and diseases affecting any tissue in a subject. In some embodiments, the disease is an ocular disease.

In certain embodiments, the condition is aging. All animals typically go through a period of growth and maturation followed by a period of progressive and irreversible physiological decline ending in death. The length of time from birth to death is known as the life span of an organism, and each organism has a characteristic average life span. Aging is a physical manifestation of the changes underlying the passage of time as measured by percent of average life span.

In some cases, characteristics of aging can be quite obvious. For example, characteristics of older humans include skin wrinkling, graying of the hair, baldness, and cataracts, as well as hypermelanosis, osteoporosis, cerebral cortical atrophy, lymphoid depletion, thymic atrophy, increased incidence of diabetes type II, atherosclerosis, cancer, and heart disease. Nehlin et al. (2000), Annals NY Acad Sci 980:176-79. Other aspects of mammalian aging include weight loss, lordokyphosis (hunchback spine), absence of vigor, lymphoid atrophy, decreased bone density, dermal thickening and subcutaneous adipose tissue, decreased ability to tolerate stress (including heat or cold, wounding, anesthesia, and hematopoietic precursor cell ablation), liver pathology, atrophy of intestinal villi, skin ulceration, amyloid deposits, and joint diseases. Tyner et al. (2002), Nature 415:45-53.

Those skilled in the art will recognize that the aging process is also manifested at the cellular level, as well as in mitochondria. Cellular aging is manifested in loss of doubling capacity, increased levels of apoptosis, changes in differentiated phenotype, and changes in metabolism, e.g., decreased levels of protein synthesis and turnover.

Given the programmed nature of cellular and organismal aging, it is possible to evaluate the “biological age” of a cell or organism by means of phenotypic characteristics that are correlated with aging. For example, biological age can be deduced from patterns of gene expression, resistance to stress (e.g., oxidative or genotoxic stress), rate of cellular proliferation, and the metabolic characteristics of cells (e.g., rates of protein synthesis and turnover, mitochondrial function, ubiquinone biosynthesis, cholesterol biosynthesis, ATP levels within the cell, levels of a Krebs cycle intermediate in the cell, glucose metabolism, nucleic acid metabolism, ribosomal translation rates, etc.). As used herein, “biological age” is a measure of the age of a cell or organism based upon the molecular characteristics of the cell or organism. Biological age is distinct from “temporal age,” which refers to the age of a cell or organism as measured by days, months, and years.

The rate of aging of an organism, e.g., an invertebrate (e.g., a worm or a fly) or a vertebrate (e.g., a rodent, e.g., a mouse) can be determined by a variety of methods, e.g., by one or more of: a) assessing the life span of the cell or the organism; (b) assessing the presence or abundance of a gene transcript or gene product in the cell or organism that has a biological age-dependent expression pattern; (c) evaluating resistance of the cell or organism to stress, e.g., genotoxic stress (e.g., etoposide, UV irradiation, exposure to a mutagen, and so forth) or oxidative stress; (d) evaluating one or more metabolic parameters of the cell or organism; (e) evaluating the proliferative capacity of the cell or a set of cells present in the organism; and (f) evaluating physical appearance or behavior of the cell or organism. In one example, evaluating the rate of aging includes directly measuring the average life span of a group of animals (e.g., a group of genetically matched animals) and comparing the resulting average to the average life span of a control group of animals (e.g., a group of animals that did not receive the test compound but are genetically matched to the group of animals that did receive the test compound). Alternatively, the rate of aging of an organism can be determined by measuring an age-related parameter. Examples of age-related parameters include: appearance, e.g., visible signs of age; the expression of one or more genes or proteins (e.g., genes or proteins that have an age-related expression pattern); resistance to oxidative stress; metabolic parameters (e.g., protein synthesis or degradation, ubiquinone biosynthesis, cholesterol biosynthesis, ATP levels, glucose metabolism, nucleic acid metabolism, ribosomal translation rates, etc.); and cellular proliferation (e.g., of retinal cells, bone cells, white blood cells, etc.).

The methods may be used to prevent or alleviate neurodegeneration and peripheral neuropathies associated. Neurodegenerative diseases include Parkinson's disease, Alzheimer's disease, multiple sclerosis, amniotropic lateral sclerosis (ALS), Huntington's disease, and muscular dystrophy. Neurodegeneration may be quantified using any method known in the art. For example, the executive function of an individual may be determined (Moreira et al., Front Aging Neurosci. 2017 Nov. 9; 9:369).

Additional age-related conditions which may be treated include heart failure, stroke, diabetes, osteoporosis, arthritis, hearing loss (partial or total), eye-related conditions (e.g., poor eye sight or retinal disease), glaucoma, and cancer. In certain embodiments, the disease is a retinal disease (e.g., macular degeneration). The condition may be a retinal disease, cancer, aging, an age-related disease, injury, or a neurodegenerative disease. In certain embodiments, the cell or tissue is from eye, ear, nose, mouth including gum and roots of teeth, bone, lung, breast, udder, pancreas, stomach, oesophagus, muscle including cardiac muscle, liver, blood vessel, skin including hair, heart, brain, nerve tissue, kidney, testis, prostate, penis, cloaca, fin, ovary, or intestine. In certain embodiments, the tissue is damaged (e.g., due to an injury, an accident, or an iatrogenic injury) and/or is aged tissue. In certain embodiments, the tissue may be considered healthy but suboptimal for performance or survival in current or future conditions (e.g., in agriculture or adverse conditions including toxic therapies, sun exposure, or travel outside the earth's atmosphere).

For example, the condition may be an injury or a disease and the condition may affect any tissue (e.g., eye, ear, bone, lung, breast, pancreas, muscle, heart, liver, skin, brain, nerve tissue, or intestine). Non-limiting examples of conditions, diseases, and disorders include acute injuries, neurodegenerative diseases, chronic diseases, cancers, aging, age-related diseases, and diseases affecting any tissue in a subject.

In some embodiments, any of the mutant rtTA4, nucleic acids (e.g., engineered nucleic acids) encoding rtTA4, nucleic acids comprising a TRE promoter operably linked to a transgene sequence, recombinant viruses, and/or recombinant cells may be used to treat a disease that affects a non-human subject (e.g., a disease affecting livestock, domesticated pets, and/or other non-human animals). For example, the disease may be a cattle disease, a primate (e.g., cynomolgus monkeys, rhesus monkeys) disease, a disease affecting a commercially relevant animal, such as cattle, pigs, horses, sheep, goats, cats, and/or dogs) and/or a disease affecting birds (e.g., commercially relevant birds, such as chickens, ducks, geese, and/or turkeys). For example, any of the, any of the mutant rtTA4, nucleic acids (e.g., engineered nucleic acids) encoding rtTA4, nucleic acids comprising a TRE promoter operably linked to a transgene sequence, recombinant viruses, and/or recombinant cells described herein may be used to promote wound healing, treat an injury (e.g., broken bones, bleeding out gun shot injury, and/or reduce scarring during surgery). In some embodiments, surgery includes cesarean.

Methods for identifying subjects suspected of having a condition may include physical examination, subject's family medical history, subject's medical history, biopsy, genetic testing, or a number of imaging technologies such as ultrasonography, computed tomography, magnetic resonance imaging, magnetic resonance spectroscopy, or positron emission tomography.

Effective amounts of the nucleic acids (e.g., engineered nucleic acids, including expression vectors), viruses (e.g., lentivirus, adenovirus, alphavirus, vaccinia virus, retrovirus, herpes virus, or AAV) or compositions thereof vary, as recognized by those skilled in the art, depending on route of administration, excipient usage, and co-usage with other active agents. The quantity to be administered depends on the subject to be treated, including, for example, the age of the subject, the gravity of the condition, the weight of the subject, the genetics of the subject, the cells, tissue, or organ to be targeted, or any combination thereof.

Using a mutant rtTA disclosed herein, expression of one or more transgenes (e.g., a therapeutic sequence, a gene-targeting nucleic acid, and/or a protein-encoding sequence) may result in reprogramming of a cell, tissue repair, tissue regeneration, organ regeneration, reversal of aging, infectious disease, prevention of a disease, treatment of a disease or any combination thereof. Cellular reprogramming may be determined by determining the extent of differentiation of a cell (e.g., by determining the expression of one or more lineage markers or pluripotency markers, including OCT4, KLF4, SOX2, NANOG, ESRRB, NR4A2, and C/EBPα). The differentiation potential of a cell may also be determined using routine differentiation assays. Tissue repair may be determined by tissue replacement and tissue regeneration assays. For example, tissue replacement assays include wound healing assays. Tissue regeneration may be determined by quantifying a particular cell type following transgene expression (e.g., expression of one or more transcription factors, including OCT4, KLF4, and SOX2) compared to before transgene expression. In some instances, the methods described herein promote organ regeneration.

These and other aspects of the present invention will be further appreciated upon consideration of the following Examples, which are intended to illustrate certain particular embodiments of the invention but are not intended to limit its scope, as defined by the claims.

### General Techniques

The practice of the present disclosure will employ, unless otherwise indicated, conventional techniques of molecular biology (including recombinant techniques), microbiology, cell biology, biochemistry and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

These and other aspects of the present invention will be further appreciated upon consideration of the following Examples, which are intended to illustrate certain particular embodiments of the invention but are not intended to limit its scope, as defined by the claims.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

### Example 1: Development of a Mutant Reverse Tetracycline Transactivator (rtTA) with Low Leakiness and Improved Reaction Time with Doxycycline Withdrawal in a Tet-on System In Vitro

A mutant reverse tetracycline transactivator (rtTA) was engineered using routine cloning techniques. An exemplary mutant rtTA (rtTA4) comprises four mutations (G12S, F67S, G72V, and R171K) as compared to rtTA3 (SEQ ID NO: 11). A rtTA comprising four mutations at positions G12, F67, G72, and R171 of rtTA3 (SEQ ID NO: 11) is referred to as rtTA4 herein. rtTA4 (SEQ ID NO: 13) comprises three VP16 transactivation domains, while rtTA3 (SEQ ID NO: 11) comprises two VP 16 transactivation domains.

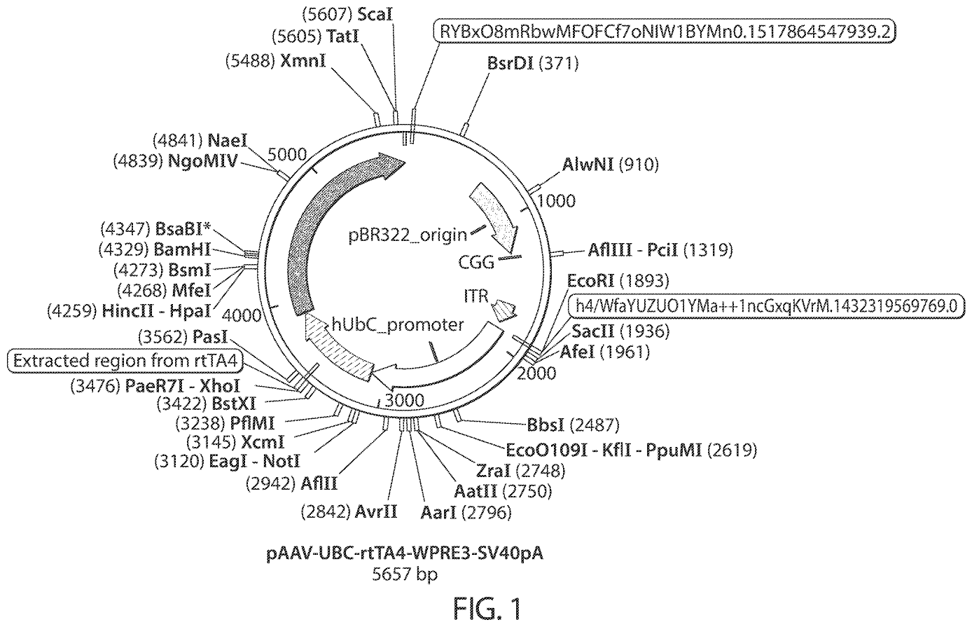

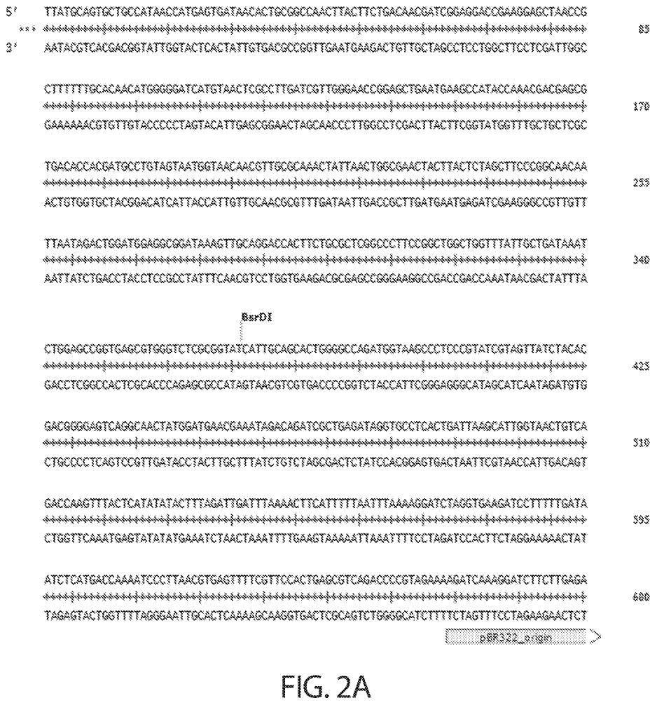

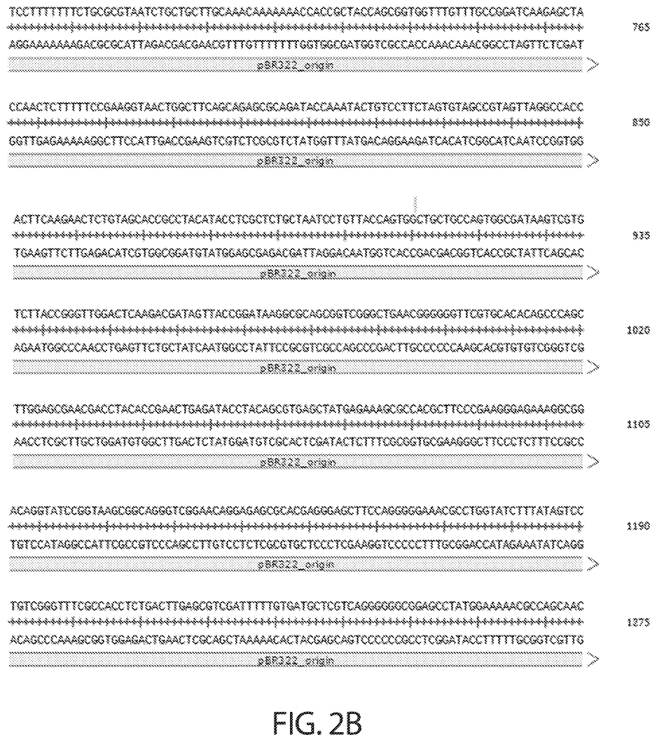

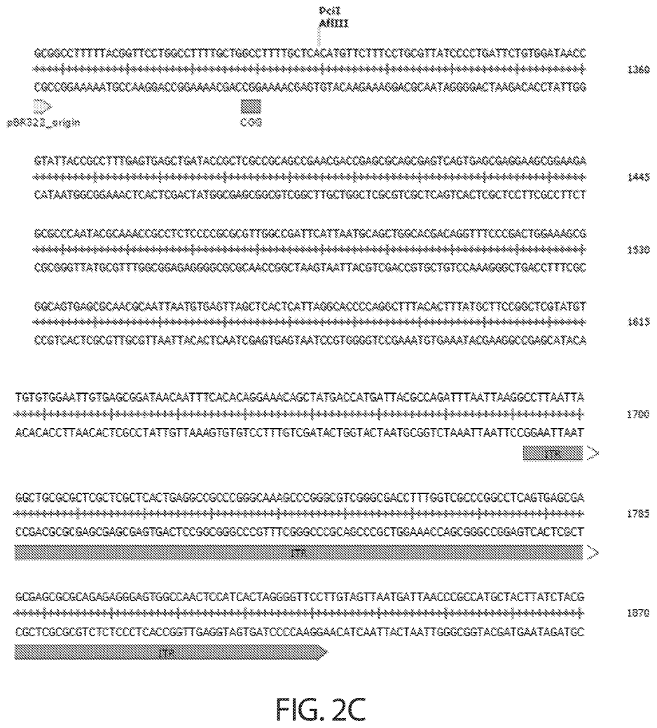

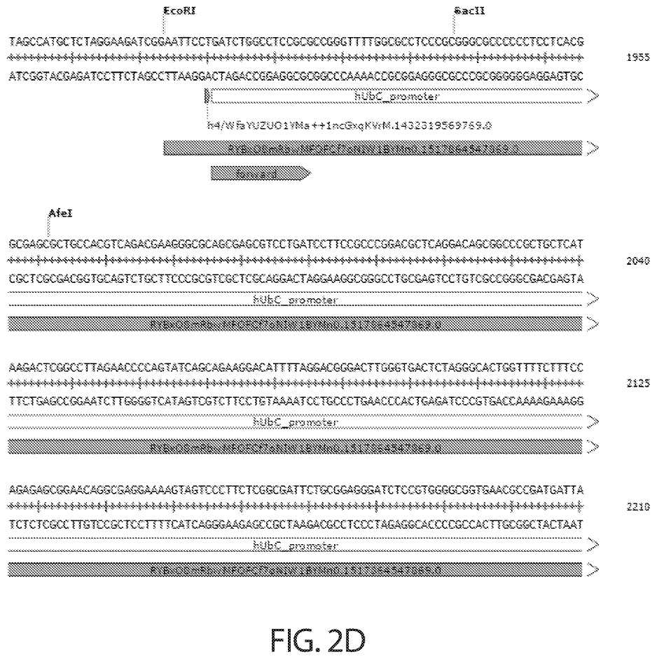

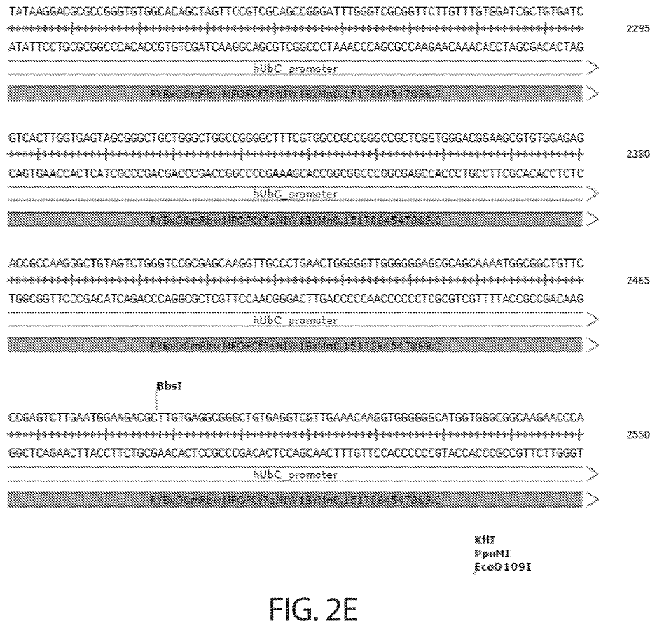

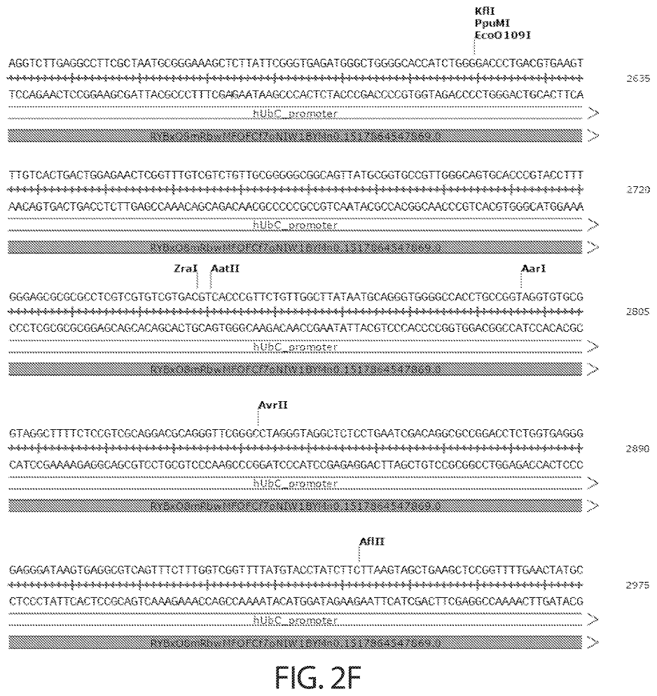

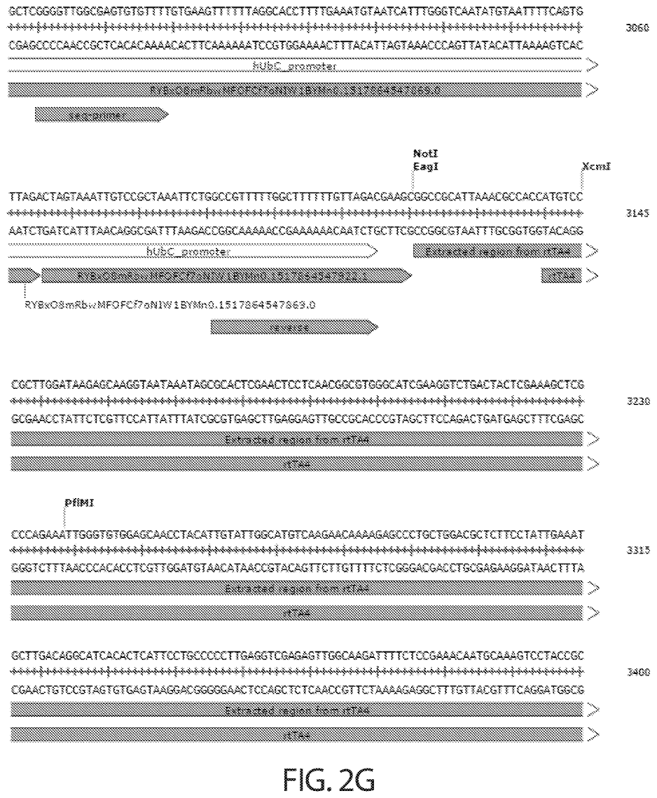

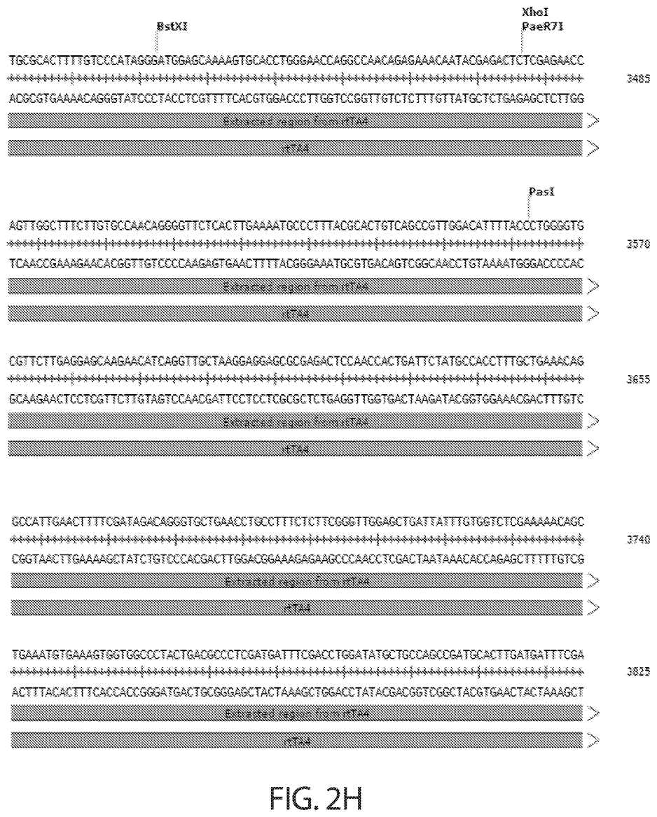

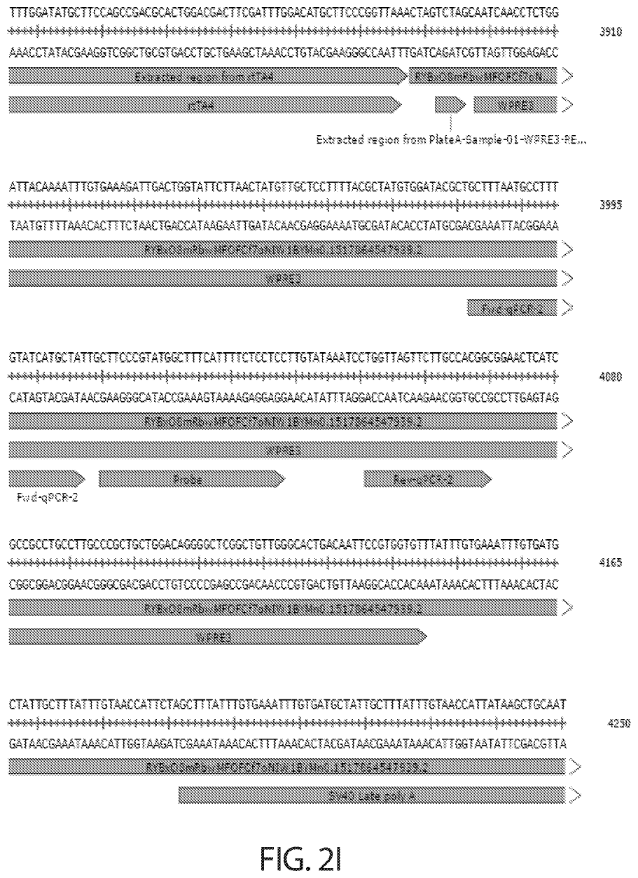

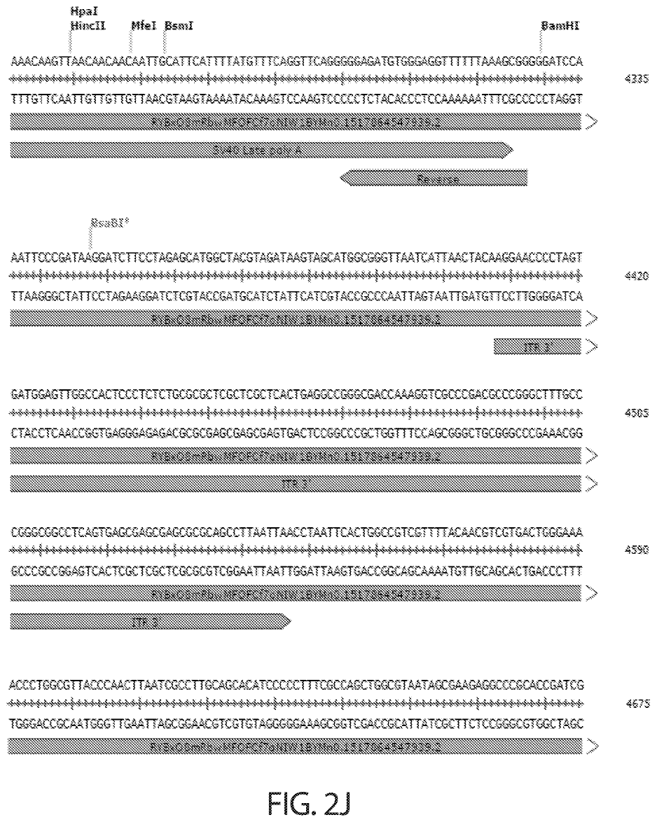

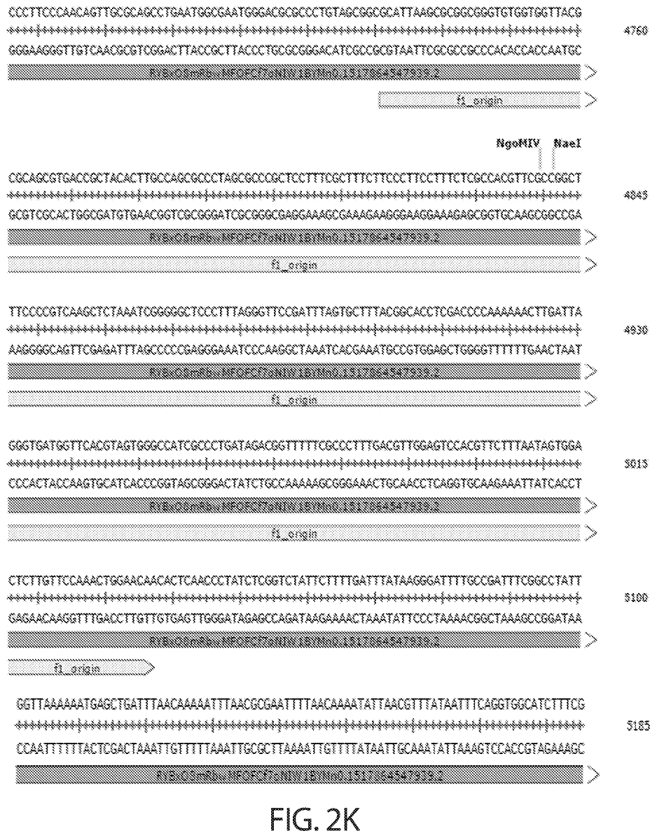

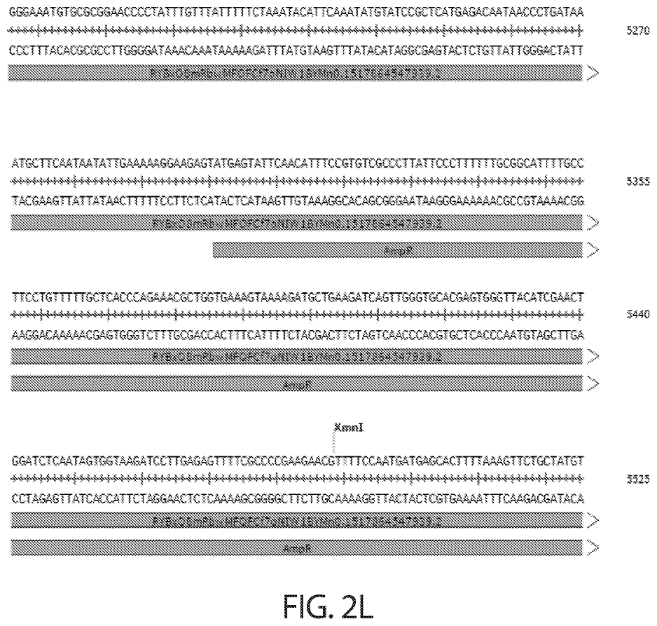

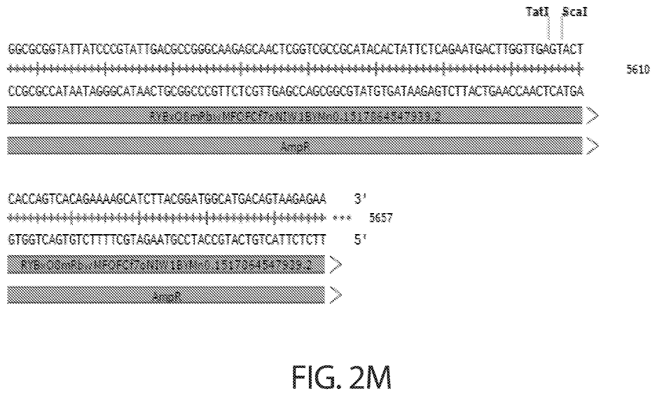

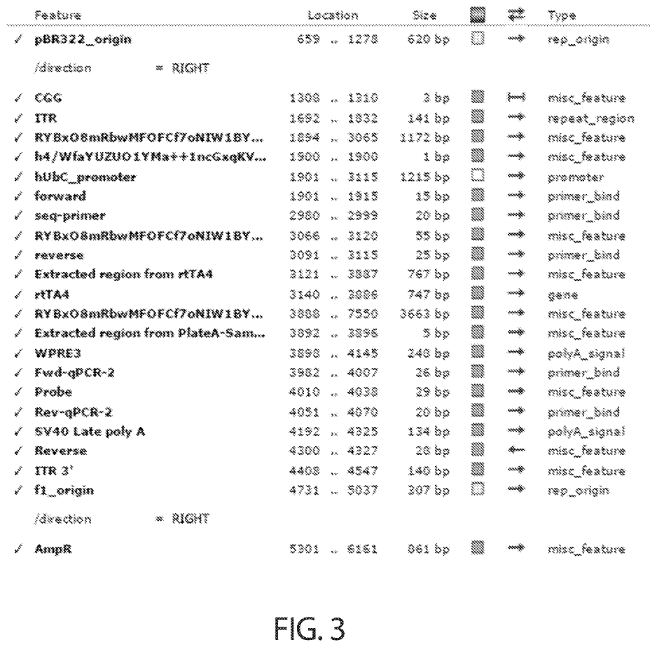

A nucleic sequence (SEQ ID NO: 12) encoding rtTA4 (SEQ ID NO: 13) was cloned into an AAV vector using routine methods. The pAAV-UBC-rtTA4-WPRE3-SV40 pA (SEQ ID NO: 17) vector is depicted in FIG. 1, FIGS. 2A-2M, and FIG. 3. FIG. 1 is a vector map showing features in an AAV vector encoding rtTA4. UBC is a constitutive promoter that is operably linked to the nucleic acid encoding rtTA4. SV40 pA is an SV40-derived terminator sequence. FIGS. 2A-2M include a series of schematics mapping the features shown in FIG. 1 onto the nucleic acid sequence of the vector encoding rtTA4. FIG. 3 shows the location and size of each feature depicted in FIGS. 2A-2M.

The pAAV-UBC-rtTA4-WPRE3-SV40 pA vector (SEQ ID NO: 17) comprises two inverted terminal repeats (ITRs) flanking a sequence encoding a UBC promoter operably linked to a nucleic acid sequence encoding rtTA4 (SEQ ID NO: 12). The AAV vector further comprises a WPRE3 sequence (SEQ ID NO: 21) and a SV40 terminator sequence (SEQ ID NO: 8). The location of restriction enzyme digestion sites are shown in Table 2 below.

TABLE 2
Restriction enzyme digestion sites in pAAV-
UBC-rtTA4-WPRE3-SV40pA (SEQ ID NO: 17).

| Enzyme | Sites | Location |
|---|---|---|
| AarI | 1 | 2796 |
| AatII | 1 | 2750 |
| AfeI | 1 | 1961 |
| AflII | 1 | 2942 |
| AflIII | 1 | 1319 |
| AlwNI | 1 | 910 |
| AvrII | 1 | 2842 |
| BamHI | 1 | 4329 |
| BbsI | 1 | 2487 |
| BsaBI | 1* | 4347* |
| BsmI | 1 | 4273 |
| BsrDI | 1 | 371 |
| BstXI | 1 | 3422 |
| EagI | 1 | 3120 |
| EcoO109I | 1 | 2619 |
| EcoRI | 1 | 1893 |
| HincII | 1 | 4259 |
| HpaI | 1 | 4259 |
| KflI | 1 | 2619 |
| MfeI | 1 | 4268 |
| NaeI | 1 | 4841 |
| NgoMIV | 1 | 4839 |
| NotI | 1 | 3120 |
| PaeR7I | 1 | 3476 |
| PasI | 1 | 3562 |
| PciI | 1 | 1319 |
| PflMI | 1 | 3238 |
| PpuMI | 1 | 2619 |
| SacII | 1 | 1936 |
| ScaI | 1 | 5607 |
| TatI | 1 | 5605 |
| XcmI | 1 | 3145 |
| XhoI | 1 | 3476 |
| XmnI | 1 | 5488 |
| ZraI | 1 | 2748 |

As shown in FIGS. 4A-4B, rtTA3 (SEQ ID NO: 11) is leaky and induced expression of a transgene that was operably linked to a tetracycline inducible promoter even in the absence of doxycycline. A nucleic acid sequence encoding rtTA3 is provided as SEQ ID NO: 10. FIG. 4A is a schematic depicting two nucleic acids that were used in this experiment. One nucleic acid encoded a UBC promoter operably linked to (1) a nucleic acid (SEQ ID NO: 12) encoding rtTA4 (SEQ ID NO: 13), (2) a nucleic acid encoding a 2A peptide, and (3) a nucleic acid encoding mKate (a far-red fluorescent protein). mKate expression was used as a readout of rtTA3 expression (FIG. 4B). The second nucleic acid encoded GFP under the control of a TRE3G (SEQ ID NO: 7) promoter. GFP should only be expressed in the presence of doxycycline (DOX) as shown in FIG. 4A.

Next, the vectors shown in FIG. 4A were tested in vivo. AAV harboring the UBC-rtTA3 vector depicted in the top portion of FIG. 4A and AAV harboring the TRE3G-GFP promoter depicted in the bottom portion of FIG. 4B were administered to mice. No AAV administration was used as a control. Mice were treated without doxycycline (DOX) or with DOX. Liver samples were then analyzed using a western blot using relevant antibodies to determine mKate expression (readout of rtTA3 expression), GFP expression, and actin expression. As shown in FIG. 4B, GFP expression was detected even in the absence of doxycycline. Therefore, rtTA3 is leaky in vivo.

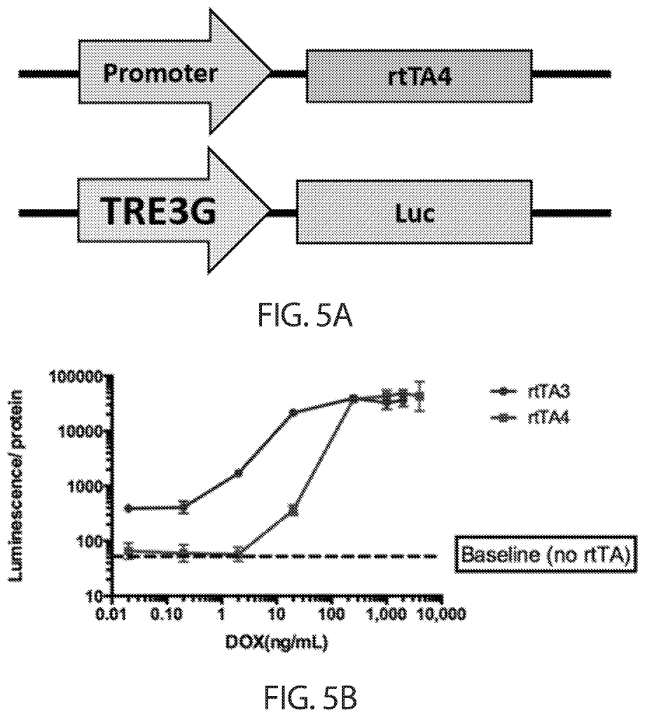

To determine whether rtTA4 is less leaky and more sensitive to doxycycline compared to rtTA3, a Tet-On luciferase reporter system was used. FIG. 5A is a schematic representation of two of the nucleic acids used in this experiment. The first nucleic acid comprises a promoter driving expression of a rtTA (FIG. 5A, top). rtTA4 is depicted in FIG. 5A, but rtTA4 was substituted with rtTA3 in the Tet-On luciferase reporter system that was used to test rtTA3. The second nucleic acid (TRE3G-Luc) comprises a TRE3G promoter operably linked to a nucleic acid sequence encoding luciferase (luc) (FIG. 5A, bottom). The Tet-On reporter system encoding rtTA4 or rtTA3 was introduced into 293T cells with the vectors alone and the cells were treated with increasing concentrations of doxycycline. Luciferase expression was determined by measuring luminescence/protein. As shown in FIG. 5B, the level of luminescence/protein was low when the level of doxycycline was low (0.01 to 1 ng/ml). The level of luminescence/protein with rtTA4 at these low levels of doxycycline was the same as baseline levels in the absence of any rtTA (baseline indicated with dotted line in FIG. 5B). The level of luminescence/protein increased with increasing levels of doxycycline in the Tet-On system with rtTA4 (FIG. 5B). In contrast, the level of luminescence/protein was significantly higher than baseline with rtTA3 even at low levels of doxycycline (e.g., 0.01 to 1 ng/ml DOX). These results suggest that rtTA4 is less leaky than rtTA3 (does not induce detectable transgene expression in the absence of doxycycline), but is capable of inducing transgene expression in the presence of increasing concentrations of doxycycline.

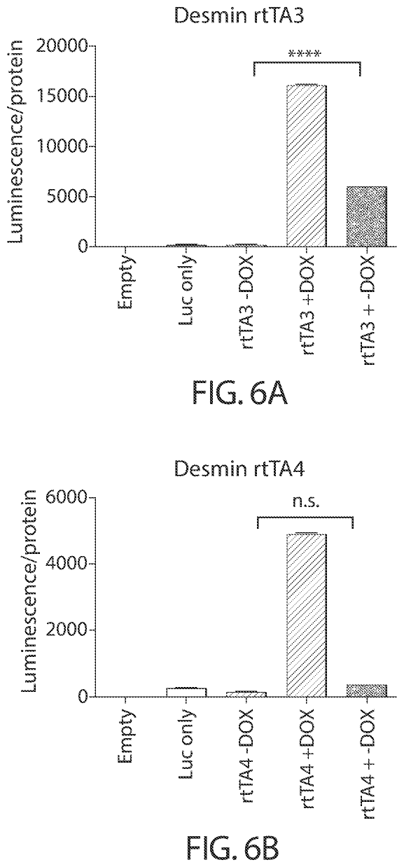

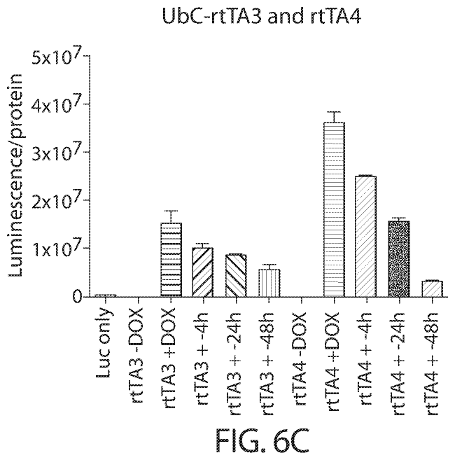

To compare the sensitivity of rtTA4 and rtTA3 to doxycycline withdrawal, Tet-On luciferase reporter systems similar to the one depicted in FIG. 5A were used. For the results shown in FIG. 6A, a TRE3G-luc vector and a vector encoding rtTA3 under the control of a desmin promoter were introduced into 293T cells. Cells received an empty vector or the TRE3G-luc vector only as controls. Cells harboring both the TRE3G-luc vector and the vector encoding rtTA3 under the desmin-promoter were treated (1) without doxycycline (−DOX), (2) with doxycycline (+DOX), or (3) with doxycycline and then doxycycline was withdrawn (+−DOX). As shown in FIG. 6A, the level of luminescence/protein detected with treatment (3) was significantly higher than the level of luminescence/protein with treatment (1). These results suggest that rtTA3 is leaky. In contrast, as shown in FIG. 6B, when the same experiment was conducted with rtTA4, doxycycline withdrawal significantly decreased the amount of luminescence/protein detected to a level that was comparable to the level detected in cells that never received doxycycline. Therefore, rtTA4 is more responsive to doxycycline withdrawal compared to rtTA3.

To compare the length of time needed for doxycycline withdrawal to turn off expression of a transgene with rtTA4 compared to rtTA3, Tet-On luciferase reporter systems comprising a TRE3G-luc vector and a vector encoding one of the rtTA proteins was introduced into 293T cells. TRE3G-luc vector was introduced into cells alone as a control. The level of luminescence/protein in the absence of any doxycycline treatment (−DOX) was also determined for the rtTA3 and for the rtTA4 system (FIG. 6C). Cells with either the rtTA3 Tet-On system or the rtTA4 Tet-On system were subsequently treated with doxycycline (+DOX), or doxycycline followed by doxycycline withdrawal (+−) for the number of hours as indicated (FIG. 6C). rtTA4 was capable of turning off transgene expression faster than rtTA3 (FIG. 6C). As shown in FIGS. 6A-6C, rtTA4 turns off 4-12 times faster than rtTA3 with both Desmin promoter and Ubc promoter.

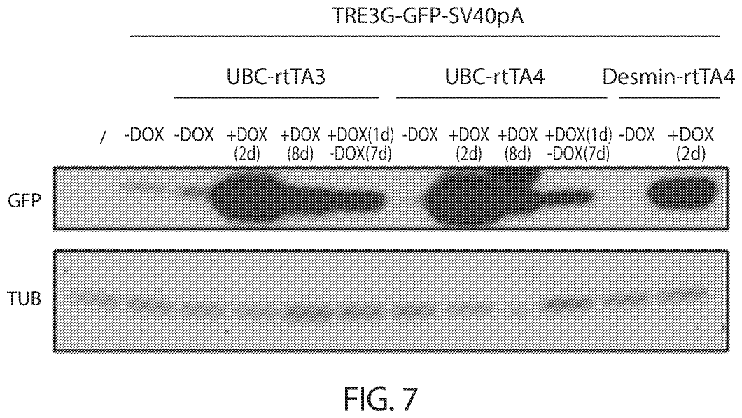

rtTA4 was further tested in Tet-On systems in mammalian 293T cells. A TRE3G-GFP-SV40 pA AAV vector and second AAV vector with (1) a sequence encoding rtTA3 operably linked to a UBC promoter, (2) a sequence encoding rtTA4 operably linked to a UBC promoter, or (3) a sequence encoding rtTA4 operably linked to a desmin promoter were introduced into 293T cells (FIG. 7). Cells were then treated in the absence of doxycycline (−DOX), in the presence of doxycycline (+DOX), or in the presence of DOX (+DOX) followed by doxycycline withdrawal (−DOX). As shown in FIG. 7, whereas rtTA3 induced GFP expression even in the absence of doxycycline, rtTA4 did not. Furthermore, removal of DOX for seven days after one day of doxycycline treatment resulted in lower GFP expression in the UBC-rtTA4 system compared to the UBC-rtTA3 system. Therefore, Tet-On systems comprising AAV vectors encoding rtTA4 successfully induced transgene expression in mammalian cells, had lower leakiness compared to the same systems with rtTA3 and showed quicker inhibition of transgene expression following doxycycline removal.

### Example 2: An AAV Vector Encoding Mutant Reverse Tetracycline Transactivator (rtTA) Showed Low Leakiness in the Liver of Mice

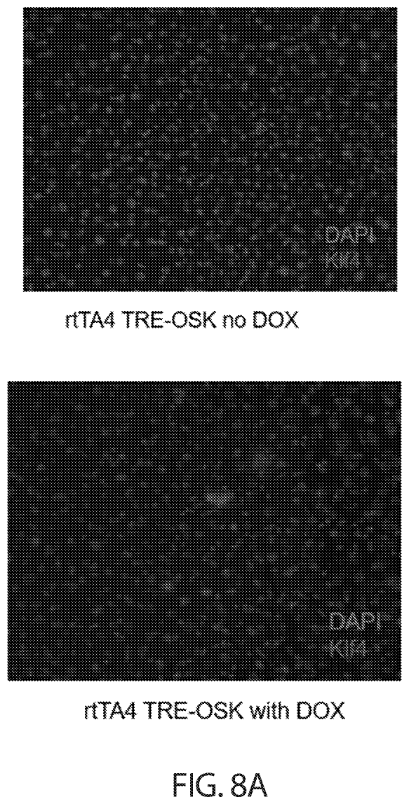

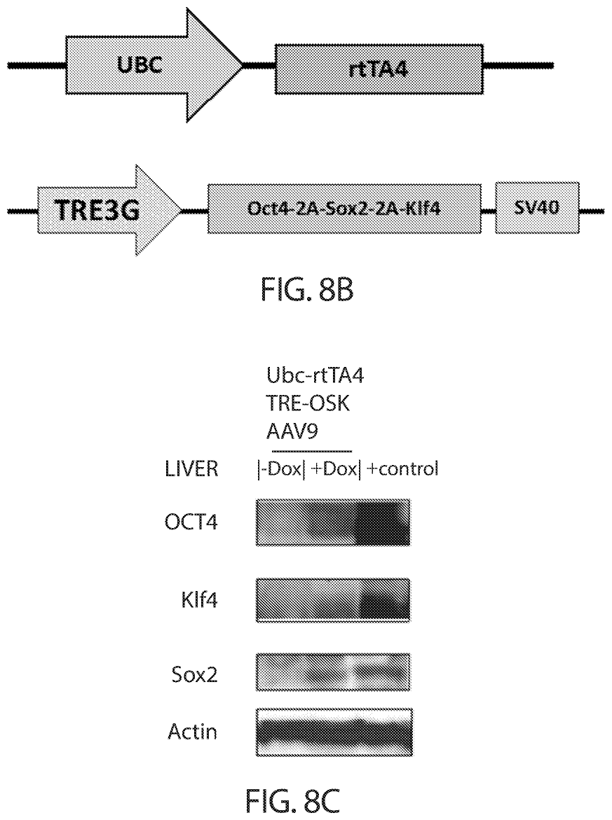

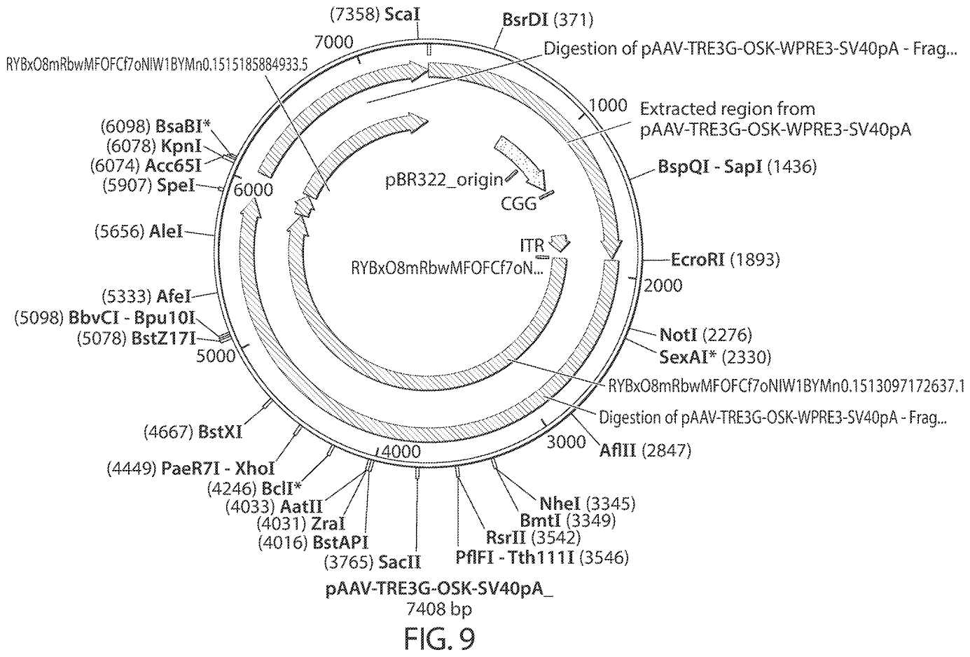

A Tet-On system comprising rtTA4 (SEQ ID NO: 13) was also tested in vivo using recombinant AAV9 viruses. Two AAV vectors comprising components shown in FIG. 8B were used. AAV virus encoding rtTA4 operably linked to a UBC promoter (pAAV-UBC-rtTA4-WPRE3-SV40 pA vector is provided as SEQ ID NO: 17) and AAV virus encoding an AAV TRE3G-OSK-SV40 pA vector (SEQ ID NO: 16) with a vector map depicted in FIG. 9 were administered to mice. Mice were treated without doxycycline or with doxycycline and liver samples were collected. As shown in the immunofluorescence images of FIG. 8A, in the absence of doxycycline, KLF4 expression was not detectable in the liver. When mice were treated with doxycycline through their drinking water, KLF4 expression was detected in the liver (FIG. 8A). These results were also evident by western blot using antibodies against OCT4, KLF4, and SOX2 to determine expression of these protein (FIG. 8C). Actin was used as a loading control (FIG. 8C). OCT4, KLF4, and SOX2 were only detected in the liver when mice were treated with doxycycline (FIG. 8C).

### Example 3: Development of a Tet-on System Combining a Tetracycline Repressor and rtTA4

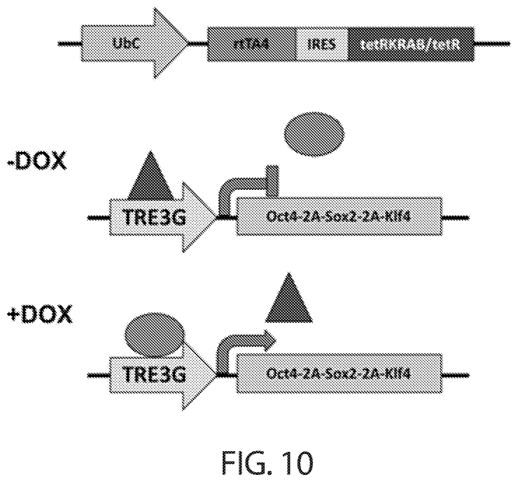

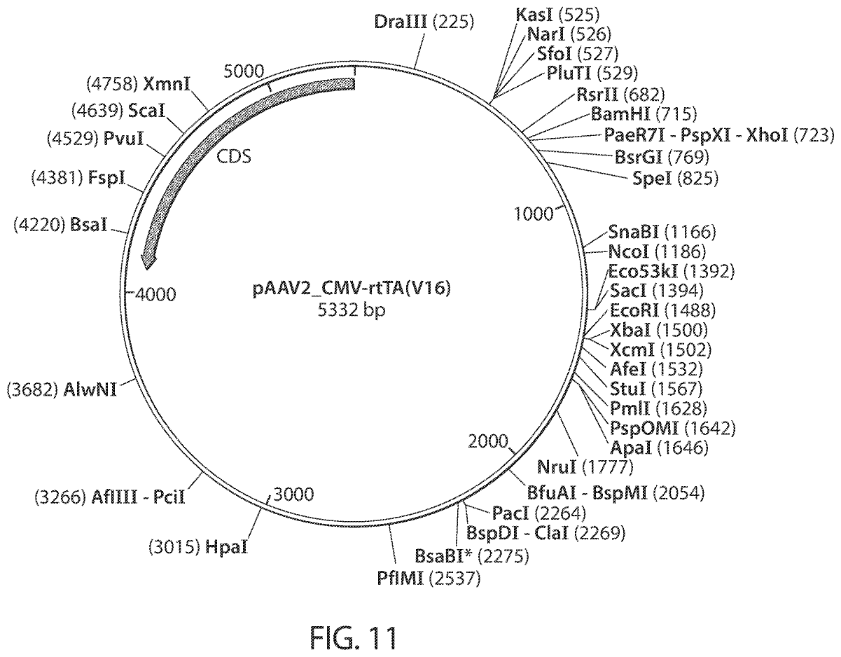

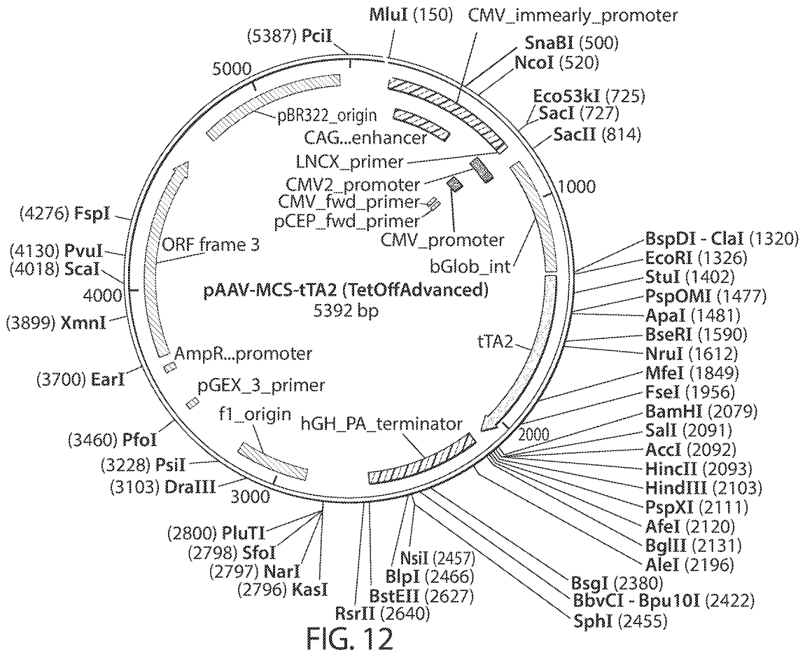

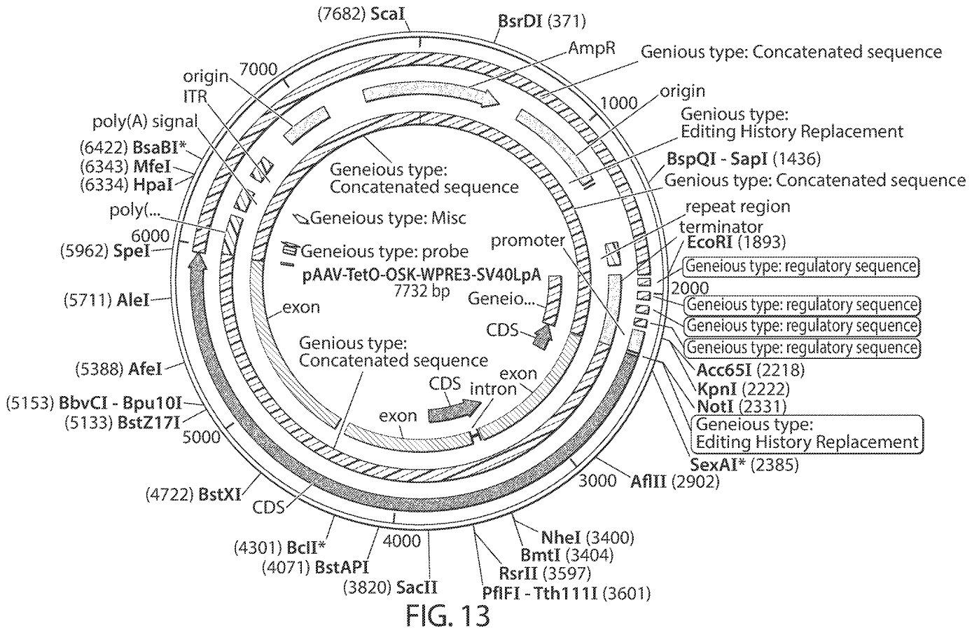

To further reduce the background binding of rtTA to TRE operator in the absence of DOX, we have developed a double insurable system of rtTA and tetR(tetRKRAB) (FIG. 10). tetR or tetRKRAB can binds to TRE operator in the absence of DOX, prevent the binding of rtTA and repress the activity of TRE. When adding DOX into system, tetR or tetRKRAB leave the TRE element and expose it to binding of rtTA to activate the expression of gene downstream of TRE. Putting this rtTA-IRES-tetRKRAB or rtTA-IRES-tetR under a continuous expressive promoter like UBC, CAG, or tissue specific promoters, allows for tight control of protein expression was achieved in a whole animal or a specific tissue.

### Example 4. Use of rtTA4 to Control Gene Expression In Vivo

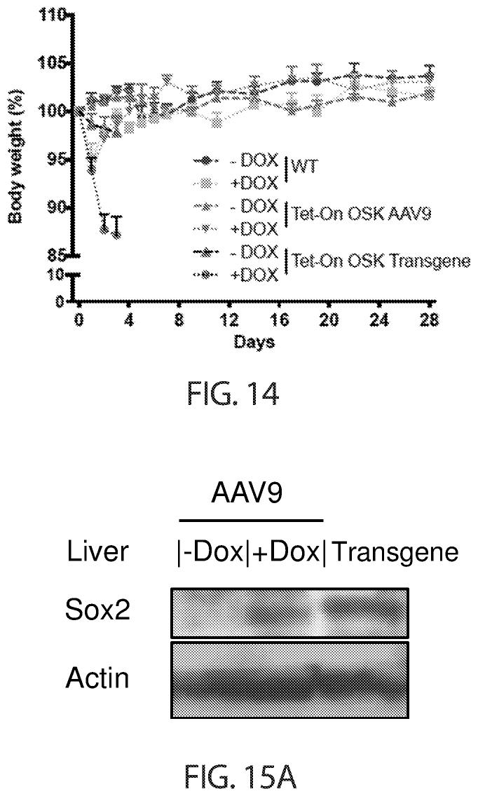

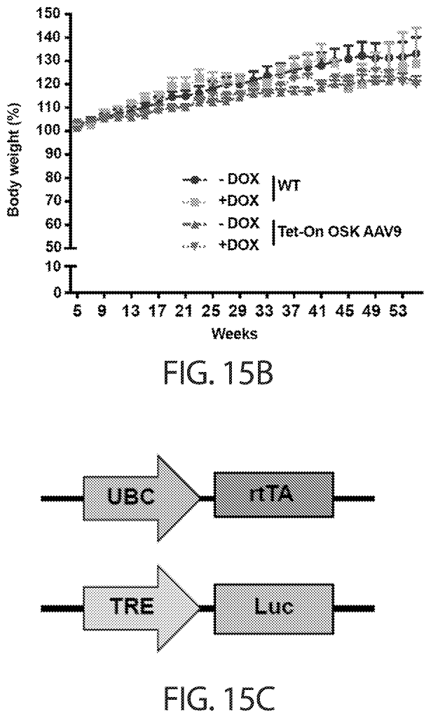

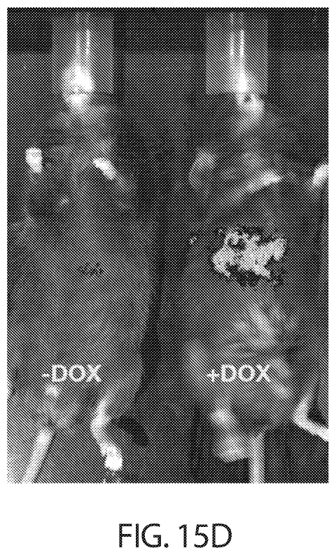

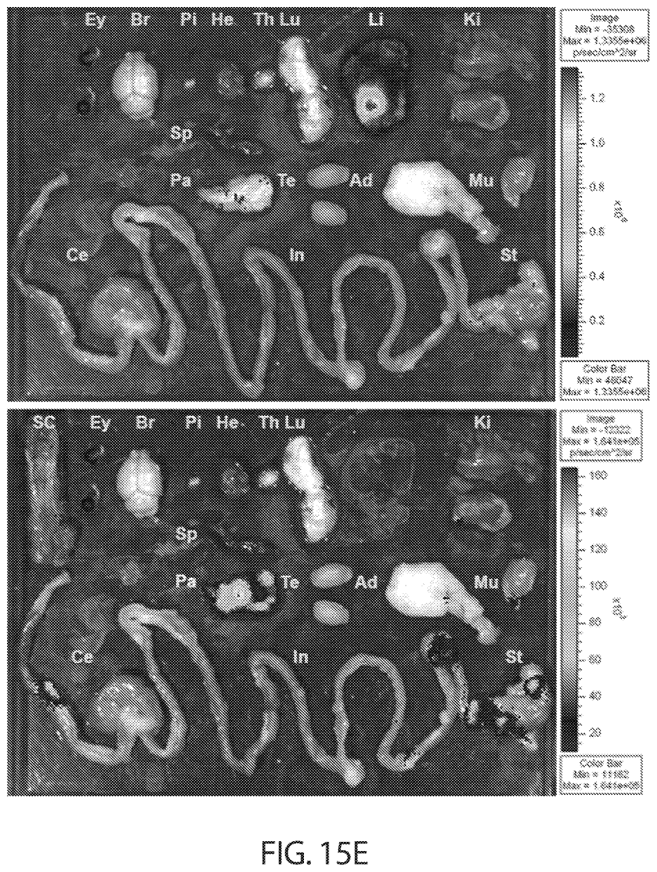

To determine whether the rtTA4 system can function in vivo, two AAVs (UBC-rtTA4 and TRE-Luc or TRE-OSK) were delivered through retro-orbital injections to mice. The 5-month-old C57BL/6J mice infected with rtTA4 and TRE-OSK AAV9s. In the absence of DOX treatment, no expression from the TRE promoter was detected (FIG. 15A). However, robust expression in tissues like liver and pancreas was observed with DOX treatment, such that the induced levels were comparable to those of transgenic mice (FIG. 15A). Surprisingly, continuous induction of OSK for over a year had no discernable negative effect on the mice for over a year (FIG. 14 and FIG. 15B). Without being bound by a particular theory, there was ostensibly no discernable negative effect on the mice because high-level expression in the intestine was avoided (FIGS. 15C-15E), thus avoiding the dysplasia and weight loss seen in other studies, including Abad et al., Nature 502, 340-345, doi:10.1038/nature12586 (2013). Therefore, the rtTA4 system allows for spatial and temporal control of gene expression in vivo.

### Methods

### Mouse Lines

C57BL6/J wild type mice are purchased from Jackson Laboratory(000664) for optic nerve crush and glaucoma model experiment. For ageing experiment, females from NIA Aged Rodent Colonies (nia.nih.gov/research/dab/aged-rodent-colonies-handbook) are used. Col1a1-tetOP-OKS-mCherry/Rosa26-M2rtTA alleles are described in Bar-Nur et al., Nat Methods, 2014. 11(11): p. 1170-6. All animal work was approved by Harvard Medical School, Boston Children's Hospital, Mass Eye and Ear Institutional animal care and use committees.

### Production of AAVs

Vectors of AAV-TRE-OSK were made by cloning mouse Oct4, Sox2 and Klf4 cDNA into an AAV plasmid consisting of the a Tet Response Element (TRE3G promoter) and SV40 element. The other vectors were directly chemically synthesized. All pAAVs, as listed in Table 6, were then packaged into AAVs of serotype 2/2 or 2/9 (titers: >5×10¹² genome copies per milliliter). Adeno associated viruses were produced by Boston Children's Hospital Viral Core.

### Systemical Delivery of AAV9 to Internal Organs

Expression in internal organs was achieved through retro-orbital injection of AAV9 (3×10¹¹ TRE-OSK plus 7×10¹¹ UBC-rtTA4). 1 mg/mL doxycycline was treated 3 weeks post injection continuously to induce OSK expression.

### Example 5. Non-Limiting Examples of Sequences

Nucleotide sequence encoding OCT4
(no stop codon): (SEQ ID NO: 1):
ATGGCTGGACACCTGGCTTCAGACTTCGCCTTCTCACCCC
CACCAGGTGGGGGTGATGGGTCAGCAGGGCTGGAGCCGGG
CTGGGTGGATCCTCGAACCTGGCTAAGCTTCCAAGGGCCT
CCAGGTGGGCCTGGAATCGGACCAGGCTCAGAGGTATTGG
GGATCTCCCCATGTCCGCCCGCATACGAGTTCTGCGGAGG
GATGGCATACTGTGGACCTCAGGTTGGACTGGGCCTAGTC
CCCCAAGTTGGCGTGGAGACTTTGCAGCCTGAGGGCCAGG
CAGGAGCACGAGTGGAAAGCAACTCAGAGGGAACCTCCTC
TGAGCCCTGTGCCGACCGCCCCAATGCCGTGAAGTTGGAG
AAGGTGGAACCAACTCCCGAGGAGTCCCAGGACATGAAAG
CCCTGCAGAAGGAGCTAGAACAGTTTGCCAAGCTGCTGAA
GCAGAAGAGGATCACCTTGGGGTACACCCAGGCCGACGTG
GGGCTCACCCTGGGCGTTCTCTTTGGAAAGGTGTTCAGCC
AGACCACCATCTGTCGCTTCGAGGCCTTGCAGCTCAGCCT
TAAGAACATGTGTAAGCTGCGGCCCCTGCTGGAGAAGTGG
GTGGAGGAAGCCGACAACAATGAGAACCTTCAGGAGATAT
GCAAATCGGAGACCCTGGTGCAGGCCCGGAAGAGAAAGCG
AACTAGCATTGAGAACCGTGTGAGGTGGAGTCTGGAGACC
ATGTTTCTGAAGTGCCCGAAGCCCTCCCTACAGCAGATCA
CTCACATCGCCAATCAGCTTGGGCTAGAGAAGGATGTGGT
TCGAGTATGGTTCTGTAACCGGCGCCAGAAGGGCAAAAGA
TCAAGTATTGAGTATTCCCAACGAGAAGAGTATGAGGCTA
CAGGGACACCTTTCCCAGGGGGGGCTGTATCCTTTCCTCT
GCCCCCAGGTCCCCACTTTGGCACCCCAGGCTATGGAAGC
CCCCACTTCACCACACTCTACTCAGTCCCTTTTCCTGAGG
GCGAGGCCTTTCCCTCTGTTCCCGTCACTGCTCTGGGCTC
TCCCATGCATTCAAAC
Amino acid sequence encoding OCT 4:
(SEQ ID NO: 2):
MAGHLASDFAFSPPPGGGDGSAGLEPGWVDPRTWLSFQGP
PGGPGIGPGSEVLGISPCPPAYEFCGGMAYCGPQVGLGLV
PQVGVETLQPEGQAGARVESNSEGTSSEPCADRPNAVKLE
KVEPTPEESQDMKALQKELEQFAKLLKQKRITLGYTQADV
GLTLGVLFGKVFSQTTICRFEALQLSLKNMCKLRPLLEKW
VEEADNNENLQEICKSETLVQARKRKRTSIENRVRWSLET
MFLKCPKPSLQQITHIANQLGLEKDVVRVWFCNRRQKGKR
SSIEYSQREEYEATGTPFPGGAVSFPLPPGPHFGTPGYGS
PHFTTLYSVPFPEGEAFPSVPVTALGSPMHSN
Nucleotide sequence encoding SOX2
(no stop codon): (SEQ ID NO: 3):
ATGTATAACATGATGGAGACGGAGCTGAAGCCGCCGGGCC
CGCAGCAAGCTTCGGGGGGCGGCGGCGGAGGAGGCAACGC
CACGGCGGCGGCGACCGGCGGCAACCAGAAGAACAGCCCG
GACCGCGTCAAGAGGCCCATGAACGCCTTCATGGTATGGT
CCCGGGGGCAGCGGCGTAAGATGGCCCAGGAGAACCCCAA
GATGCACAACTCGGAGATCAGCAAGCGCCTGGGCGCGGAG
TGGAAACTTTTGTCCGAGACCGAGAAGCGGCCGTTCATCG
ACGAGGCCAAGCGGCTGCGCGCTCTGCACATGAAGGAGCA
CCCGGATTATAAATACCGGCCGCGGCGGAAAACCAAGACG
CTCATGAAGAAGGATAAGTACACGCTTCCCGGAGGCTTGC
TGGCCCCCGGCGGGAACAGCATGGCGAGCGGGGTTGGGGT
GGGCGCCGGCCTGGGTGCGGGCGTGAACCAGCGCATGGAC
AGCTACGCGCACATGAACGGCTGGAGCAACGGCAGCTACA
GCATGATGCAGGAGCAGCTGGGCTACCCGCAGCACCCGGG
CCTCAACGCTCACGGCGCGGCACAGATGCAACCGATGCAC
CGCTACGACGTCAGCGCCCTGCAGTACAACTCCATGACCA
GCTCGCAGACCTACATGAACGGCTCGCCCACCTACAGCAT
GTCCTACTCGCAGCAGGGCACCCCCGGTATGGCGCTGGGC
TCCATGGGCTCTGTGGTCAAGTCCGAGGCCAGCTCCAGCC
CCCCCGTGGTTACCTCTTCCTCCCACTCCAGGGCGCCCTG
CCAGGCCGGGGACCTCCGGGACATGATCAGCATGTACCTC
CCCGGCGCCGAGGTGCCGGAGCCCGCTGCGCCCAGTAGAC
TGCACATGGCCCAGCACTACCAGAGCGGCCCGGTGCCCGG
CACGGCCATTAACGGCACACTGCCCCTGTCGCACATG
Amino acid sequence encoding SOX2
(translated): (SEQ ID NO: 4)
MYNMMETELKPPGPQQASGGGGGGGNATAAATGGNQKNSP
DRVKRPMNAFMVWSRGQRRKMAQENPKMHNSEISKRLGAE
WKLLSETEKRPFIDEAKRLRALHMKEHPDYKYRPRRKTKT
LMKKDKYTLPGGLLAPGGNSMASGVGVGAGLGAGVNQRMD
SYAHMNGWSNGSYSMMQEQLGYPQHPGLNAHGAAQMQPMH
RYDVSALQYNSMTSSQTYMNGSPTYSMSYSQQGTPGMALG
SMGSVVKSEASSSPPVVTSSSHSRAPCQAGDLRDMISMYL
PGAEVPEPAAPSRLHMAQHYQSGPVPGTAINGTLPLSHM
Nucleotide sequence encoding KLF4
(no stop codon): (SEQ ID NO: 5):
ATGAGGCAGCCACCTGGCGAGTCTGACATGGCTGTCAGCG
ACGCTCTGCTCCCGTCCTTCTCCACGTTCGCGTCCGGCCC
GGCGGGAAGGGAGAAGACACTGCGTCCAGCAGGTGCCCCG
ACTAACCGTTGGCGTGAGGAACTCTCTCACATGAAGCGAC
TTCCCCCACTTCCCGGCCGCCCCTACGACCTGGCGGCGAC
GGTGGCCACAGACCTGGAGAGTGGCGGAGCTGGTGCAGCT
TGCAGCAGTAACAACCCGGCCCTCCTAGCCCGGAGGGAGA
CCGAGGAGTTCAACGACCTCCTGGACCTAGACTTTATCCT
TTCCAACTCGCTAACCCACCAGGAATCGGTGGCCGCCACC
GTGACCACCTCGGCGTCAGCTTCATCCTCGTCTTCCCCAG
CGAGCAGCGGCCCTGCCAGCGCGCCCTCCACCTGCAGCTT
CAGCTATCCGATCCGGGCCGGGGGTGACCCGGGCGTGGCT
GCCAGCAACACAGGTGGAGGGCTCCTCTACAGCCGAGAAT
CTGCGCCACCTCCCACGGCCCCCTTCAACCTGGCGGACAT
CAATGACGTGAGCCCCTCGGGCGGCTTCGTGGCTGAGCTC
CTGCGGCCGGAGTTGGACCCAGTATACATTCCGCCACAGC
AGCCTCAGCCGCCAGGTGGCGGGCTGATGGGCAAGTTTGT
GCTGAAGGCGTCTCTGACCACCCCTGGCAGCGAGTACAGC
AGCCCTTCGGTCATCAGTGTTAGCAAAGGAAGCCCAGACG
GCAGCCACCCCGTGGTAGTGGCGCCCTACAGCGGTGGCCC
GCCGCGCATGTGCCCCAAGATTAAGCAAGAGGCGGTCCCG
TCCTGCACGGTCAGCCGGTCCCTAGAGGCCCATTTGAGCG
CTGGACCCCAGCTCAGCAACGGCCACCGGCCCAACACACA
CGACTTCCCCCTGGGGCGGCAGCTCCCCACCAGGACTACC
CCTACACTGAGTCCCGAGGAACTGCTGAACAGCAGGGACT
GTCACCCTGGCCTGCCTCTTCCCCCAGGATTCCATCCCCA
TCCGGGGCCCAACTACCCTCCTTTCCTGCCAGACCAGATG
CAGTCACAAGTCCCCTCTCTCCATTATCAAGAGCTCATGC
CACCGGGTTCCTGCCTGCCAGAGGAGCCCAAGCCAAAGAG
GGGAAGAAGGTCGTGGCCCCGGAAAAGAACAGCCACCCAC
ACTTGTGACTATGCAGGCTGTGGCAAAACCTATACCAAGA
GTTCTCATCTCAAGGCACACCTGCGAACTCACACAGGCGA
GAAACCTTACCACTGTGACTGGGACGGCTGTGGGTGGAAA
TTCGCCCGCTCCGATGAACTGACCAGGCACTACCGCAAAC
ACACAGGGCACCGGCCCTTTCAGTGCCAGAAGTGCGACAG
GGCCTTTTCCAGGTCGGACCACCTTGCCTTACACATGAAG
AGGCAC
Amino acid sequence encoding KLF4
(translated): (SEQ ID NO: 6):
MRQPPGESDMAVSDALLPSFSTFASGPAGREKTLRPAGAP
TNRWREELSHMKRLPPLPGRPYDLAATVATDLESGGAGAA
CSSNNPALLARRETEEFNDLLDLDFILSNSLTHQESVAAT
VTTSASASSSSSPASSGPASAPSTCSFSYPIRAGGDPGVA
ASNTGGGLLYSRESAPPPTAPFNLADINDVSPSGGFVAEL
LRPELDPVYIPPQQPQPPGGGLMGKFVLKASLTTPGSEYS
SPSVISVSKGSPDGSHPVVVAPYSGGPPRMCPKIKQEAVP
SCTVSRSLEAHLSAGPQLSNGHRPNTHDFPLGRQLPTRTT
PTLSPEELLNSRDCHPGLPLPPGFHPHPGPNYPPFLPDQM
QSQVPSLHYQELMPPGSCLPEEPKPKRGRRSWPRKRTATH
TCDYAGCGKTYTKSSHLKAHLRTHTGEKPYHCDWDGCGWK
FARSDELTRHYRKHTGHRPFQCQKCDRAFSRSDHLALHMK
RH
TRE3G promoter sequence (non-limiting
example of a TRE promoter):
(SEQ ID NO: 7):
TTTACTCCCTATCAGTGATAGAGAACGTATGAAGAGTTTA
CTCCCTATCAGTGATAGAGAACGTATGCAGACTTTACTCC
CTATCAGTGATAGAGAACGTATAAGGAGTTTACTCCCTAT
CAGTGATAGAGAACGTATGACCAGTTTACTCCCTATCAGT
GATAGAGAACGTATCTACAGTTTACTCCCTATCAGTGATA
GAGAACGTATATCCAGTTTACTCCCTATCAGTGATAGAGA
ACGTATAAGCTTTAGGCGTGTACGGTGGGCGCCTATAAAA
GCAGAGCTCGTTTAGTGAACCGTCAGATCGCCTGGAGCAA
TTCCACAACACTTTTGTCTTATACCAACTTTCCGTACCAC
TTCCTACCCTCGTAAA
5V40-derived terminator sequence:
(SEQ ID NO: 8):
TGCGCGCAGCGGCCGACCATGGCCCAACTTGTTTATTGCA
GCTTATAATGGTTACAAATAAAGCAATAGCATCACAAATT
TCACAAATAAAGCATTTTTTTCACTGCATTCTAGTTGTGG
TTTGTCCAAACTCATCAATGTATCTTATCATGTCTGGATC
TCGGTACCG
T2A sequence (SEQ ID NO: 9):
GSGEGRGSLLTCGDVEENPGP
Nucleotide sequence encoding rtTA3
(with 2 VP16 domain at 3′ end):
(SEQ ID NO: 10):
ATGTCTAGGCTGGACAAGAGCAAAGTCATAAACGGAGCTC
TGGAATTACTCAATGGTGTCGGTATCGAAGGCCTGACGAC
AAGGAAACTCGCTCAAAAGCTGGGAGTTGAGCAGCCTACC
CTGTACTGGCACGTGAAGAACAAGCGGGCCCTGCTCGATG
CCCTGCCAATCGAGATGCTGGACAGGCATCATACCCACTT
CTGCCCCCTGGAAGGCGAGTCATGGCAAGACTTTCTGCGG
AACAACGCCAAGTCATACCGCTGTGCTCTCCTCTCACATC
GCGACGGGGCTAAAGTGCATCTCGGCACCCGCCCAACAGA
GAAACAGTACGAAACCCTGGAAAATCAGCTCGCGTTCCTG
TGTCAGCAAGGCTTCTCCCTGGAGAACGCACTGTACGCTC
TGTCCGCCGTGGGCCACTTTACACTGGGCTGCGTATTGGA
GGAACAGGAGCATCAAGTAGCAAAAGAGGAAAGAGAGACA
CCTACCACCGATTCTATGCCCCCACTTCTGAGACAAGCAA
TTGAGCTGTTCGACCGGCAGGGAGCCGAACCTGCCTTCCT
TTTCGGCCTGGAACTAATCATATGTGGCCTGGAGAAACAG
CTAAAGTGCGAAAGCGGCGGGCCGACCGACGCCCTTGACG
ATTTTGACTTAGACATGCTCCCAGCCGATGCCCTTGACGA
TTTTGACCTTGACATGCTCCCCGGGTAA
Amino acid sequence encoding rtTA3:
(SEQ ID NO: 11):
MSRLDKSKVINGALELLNGVGIEGLTTRKLAQKLGVEQPT
LYWHVKNKRALLDALPIEMLDRHHTHFCPLEGESWQDFLR
NNAKSYRCALLSHRDGAKVHLGTRPTEKQYETLENQLAFL
CQQGFSLENALYALSAVGHFTLGCVLEEQEHQVAKEERET
PTTDSMPPLLRQAIELFDRQGAEPAFLFGLELIICGLEKQ
LKCESGGPTDALDDFDLDMLPADALDDFDLDMLPG
Nucleotide sequence encoding rtTA4
(with 3 VP16 domain at 3′ end):
(SEQ ID NO: 12):
ATGTCCCGCTTGGATAAGAGCAAGGTAATAAATAGCGCAC
TCGAACTCCTCAACGGCGTGGGCATCGAAGGTCTGACTAC
TCGAAAGCTCGCCCAGAAATTGGGTGTGGAGCAACCTACA
TTGTATTGGCATGTCAAGAACAAAAGAGCCCTGCTGGACG
CTCTTCCTATTGAAATGCTTGACAGGCATCACACTCATTC
CTGCCCCCTTGAGGTCGAGAGTTGGCAAGATTTTCTCCGA
AACAATGCAAAGTCCTACCGCTGCGCACTTTTGTCCCATA
GGGATGGAGCAAAAGTGCACCTGGGAACCAGGCCAACAGA
GAAACAATACGAGACTCTCGAGAACCAGTTGGCTTTCTTG
TGCCAACAGGGGTTCTCACTTGAAAATGCCCTTTACGCAC
TGTCAGCCGTTGGACATTTTACCCTGGGGTGCGTTCTTGA
GGAGCAAGAACATCAGGTTGCTAAGGAGGAGCGCGAGACT
CCAACCACTGATTCTATGCCACCTTTGCTGAAACAGGCCA
TTGAACTTTTCGATAGACAGGGTGCTGAACCTGCCTTTCT
CTTCGGGTTGGAGCTGATTATTTGTGGTCTCGAAAAACAG
CTGAAATGTGAAAGTGGTGGCCCTACTGACGCCCTCGATG
ATTTCGACCTGGATATGCTGCCAGCCGATGCACTTGATGA
TTTCGATTTGGATATGCTTCCAGCCGACGCACTGGACGAC
TTCGATTTGGACATGCTTCCCGGTTAA
Amino acid sequence encoding rtTA4:
(SEQ ID NO: 13):
MSRLDKSKVINSALELLNGVGIEGLTTRKLAQKLGVEQPT
LYWHVKNKRALLDALPIEMLDRHHTHSCPLEVESWQDFLR
NNAKSYRCALLSHRDGAKVHLGTRPTEKQYETLENQLAFL
CQQGFSLENALYALSAVGHFTLGCVLEEQEHQVAKEERET
PTTDSMPPLLKQAIELFDRQGAEPAFLFGLELIICGLEKQ
LKCESGGPTDALDDFDLDMLPADALDDFDLDMLPADALDD
FDLDMLPG
Nucleotide sequence encoding
M2-rtTA (SEQ ID NO: 14):
ATGCCTTTGTATCATGCTATTGCTTCCCGTATGGCTTTCA
TTTTCTCCTCCTTGTATAAATCCTGGTTGCTGTCTCTTTA
TGAGGAGTTGTGGCCCGTTGTCAGGCAACGTGGCGTGGTG
TGCACTGTGTTTGCTGACGCAACCCCCACTGGTTGGGGCA
TTGCCACCACCTGTCAGCTCCTTTCCGGGACTTTCGCTTT
CCCCCTCCCTATTGCCACGGCGGAACTCATCGCCGCCTGC
CTTGCCCGCTGCTGGACAGGGGCTCGGCTGTTGGGCACTG
ACAATTCCGTGGTGTTGTCGGGGAAATCATCGTCCTTTCC
TTGGCTGCTCGCCTGTGTTGCCACCTGGATTCTGCGCGGG
ACGTCCTTCTGCTACGTCCCTTCGGCCCTCAATCCAGCGG
ACCTTCCTTCCCGCGGCCTGCTGCCGGCTCTGCGGCCTCT
TCCGCGTCTTCGCCTTCGCCCTCAGACGAGTCGGATCTCC
CTTTGGGCCGCCTCCCCGCATCGATACCGTCGACCTCGAG
ACCTAGAAAAACATGGAGCAATCACAAGTAGCAATACAGC
AGCTACCAATGCTGATTGTGCCTGGCTAGAAGCACAAGAG
GAGGAGGAGGTGGGTTTTCCAGTCACACCTCAGGTACCTT
TAAGACCAATGACTTACAAGGCAGCTGTAGATCTTAGCCA
CTTTTTAAAAGAAAAGGGGGGACTGGAAGGGCTAATTCAC
TCCCAACGAAGACAAGATATCCTTGATCTGTGGATCTACC
ACACACAAGGCTACTTCCCTGATTGGCAGAACTACACACC
AGGGCCAGGGATCAGATATCCACTGACCTTTGGATGGTGC
TACAAGCTAGTACCAGTTGAGCAAGAGAAGGTAGAAGAAG
CCAATGAAGGAGAGAACACCCGCTTGTTACACCCTGTGAG
CCTGCATGGGATGGATGACCCGGAGAGAGAAGTATTAGAG
TGGAGGTTTGACAGCCGCCTAGCATTTCATCACATGGCCC
GAGAGCTGCATCCGGACTGTACTGGGTCTCTCTGGTTAGA
CCAGATCTGA
Amino acid sequence encoding
M2-rtTA (SEQ ID NO: 15):
MPLYHAIASRMAFIFSSLYKSWLLSLYEELWPVVRQRGVV
CTVFADATPTGWGIATTCQLLSGTFAFPLPIATAELIAAC
LARCWTGARLLGTDNSVVLSGKSSSFPWLLACVATWILRG
TSFCYVPSALNPADLPSRGLLPALRPLPRLRLRPQTSRIS
LWAASPHRYRRPRDLEKHGAITSSNTAATNADCAWLEAQE
EEEVGFPVTPQVPLRPMTYKAAVDLSHFLKEKGGLEGLIH
SQRRQDILDLWIYHTQGYFPDWQNYTPGPGIRYPLTFGWC
YKLVPVEQEKVEEANEGENTRLLHPVSLHGMDDPEREVLE
WRFDSRLAFHHMARELHPDCTGSLWLDQI
Nucleic acid sequence of
pAAV-TRE3G-OSK-SV40pA
or TRE3G-OSK-SV40pA vector
(SEQ ID NO: 16):
TTATGCAGTGCTGCCATAACCATGAGTGATAACACTGCGG
CCAACTTACTTCTGACAACGATCGGAGGACCGAAGGAGCT
AACCGCTTTTTTGCACAACATGGGGGATCATGTAACTCGC
CTTGATCGTTGGGAACCGGAGCTGAATGAAGCCATACCAA
ACGACGAGCGTGACACCACGATGCCTGTAGTAATGGTAAC
AACGTTGCGCAAACTATTAACTGGCGAACTACTTACTCTA
GCTTCCCGGCAACAATTAATAGACTGGATGGAGGCGGATA
AAGTTGCAGGACCACTTCTGCGCTCGGCCCTTCCGGCTGG
CTGGTTTATTGCTGATAAATCTGGAGCCGGTGAGCGTGGG
TCTCGCGGTATCATTGCAGCACTGGGGCCAGATGGTAAGC
CCTCCCGTATCGTAGTTATCTACACGACGGGGAGTCAGGC
AACTATGGATGAACGAAATAGACAGATCGCTGAGATAGGT
GCCTCACTGATTAAGCATTGGTAACTGTCAGACCAAGTTT
ACTCATATATACTTTAGATTGATTTAAAACTTCATTTTTA
ATTTAAAAGGATCTAGGTGAAGATCCTTTTTGATAATCTC
ATGACCAAAATCCCTTAACGTGAGTTTTCGTTCCACTGAG
CGTCAGACCCCGTAGAAAAGATCAAAGGATCTTCTTGAGA
TCCTTTTTTTCTGCGCGTAATCTGCTGCTTGCAAACAAAA
AAACCACCGCTACCAGCGGTGGTTTGTTTGCCGGATCAAG
AGCTACCAACTCTTTTTCCGAAGGTAACTGGCTTCAGCAG
AGCGCAGATACCAAATACTGTCCTTCTAGTGTAGCCGTAG
TTAGGCCACCACTTCAAGAACTCTGTAGCACCGCCTACAT
ACCTCGCTCTGCTAATCCTGTTACCAGTGGCTGCTGCCAG
TGGCGATAAGTCGTGTCTTACCGGGTTGGACTCAAGACGA
TAGTTACCGGATAAGGCGCAGCGGTCGGGCTGAACGGGGG
GTTCGTGCACACAGCCCAGCTTGGAGCGAACGACCTACAC
CGAACTGAGATACCTACAGCGTGAGCTATGAGAAAGCGCC
ACGCTTCCCGAAGGGAGAAAGGCGGACAGGTATCCGGTAA
GCGGCAGGGTCGGAACAGGAGAGCGCACGAGGGAGCTTCC
AGGGGGAAACGCCTGGTATCTTTATAGTCCTGTCGGGTTT
CGCCACCTCTGACTTGAGCGTCGATTTTTGTGATGCTCGT
CAGGGGGGCGGAGCCTATGGAAAAACGCCAGCAACGCGGC
CTTTTTACGGTTCCTGGCCTTTTGCTGGCCTTTTGCTCAC
ATGTTCTTTCCTGCGTTATCCCCTGATTCTGTGGATAACC
GTATTACCGCCTTTGAGTGAGCTGATACCGCTCGCCGCAG
CCGAACGACCGAGCGCAGCGAGTCAGTGAGCGAGGAAGCG
GAAGAGCGCCCAATACGCAAACCGCCTCTCCCCGCGCGTT
GGCCGATTCATTAATGCAGCTGGCACGACAGGTTTCCCGA
CTGGAAAGCGGGCAGTGAGCGCAACGCAATTAATGTGAGT
TAGCTCACTCATTAGGCACCCCAGGCTTTACACTTTATGC
TTCCGGCTCGTATGTTGTGTGGAATTGTGAGCGGATAACA
ATTTCACACAGGAAACAGCTATGACCATGATTACGCCAGA
TTTAATTAAGGCCTTAATTAGGCTGCGCGCTCGCTCGCTC
ACTGAGGCCGCCCGGGCAAAGCCCGGGCGTCGGGCGACCT
TTGGTCGCCCGGCCTCAGTGAGCGAGCGAGCGCGCAGAGA
GGGAGTGGCCAACTCCATCACTAGGGGTTCCTTGTAGTTA
ATGATTAACCCGCCATGCTACTTATCTACGTAGCCATGCT
CTAGGAAGATCGGAATTCTTTACTCCCTATCAGTGATAGA
GAACGTATGAAGAGTTTACTCCCTATCAGTGATAGAGAAC
GTATGCAGACTTTACTCCCTATCAGTGATAGAGAACGTAT
AAGGAGTTTACTCCCTATCAGTGATAGAGAACGTATGACC
AGTTTACTCCCTATCAGTGATAGAGAACGTATCTACAGTT
TACTCCCTATCAGTGATAGAGAACGTATATCCAGTTTACT
CCCTATCAGTGATAGAGAACGTATAAGCTTTAGGCGTGTA
CGGTGGGCGCCTATAAAAGCAGAGCTCGTTTAGTGAACCG
TCAGATCGCCTGGAGCAATTCCACAACACTTTTGTCTTAT
ACCAACTTTCCGTACCACTTCCTACCCTCGTAAAGCGGCC
GCGCCACCATGGCTGGACACCTGGCTTCAGACTTCGCCTT
CTCACCCCCACCAGGTGGGGGTGATGGGTCAGCAGGGCTG
GAGCCGGGCTGGGTGGATCCTCGAACCTGGCTAAGCTTCC
AAGGGCCTCCAGGTGGGCCTGGAATCGGACCAGGCTCAGA
GGTATTGGGGATCTCCCCATGTCCGCCCGCATACGAGTTC
TGCGGAGGGATGGCATACTGTGGACCTCAGGTTGGACTGG
GCCTAGTCCCCCAAGTTGGCGTGGAGACTTTGCAGCCTGA
GGGCCAGGCAGGAGCACGAGTGGAAAGCAACTCAGAGGGA
ACCTCCTCTGAGCCCTGTGCCGACCGCCCCAATGCCGTGA
AGTTGGAGAAGGTGGAACCAACTCCCGAGGAGTCCCAGGA
CATGAAAGCCCTGCAGAAGGAGCTAGAACAGTTTGCCAAG
CTGCTGAAGCAGAAGAGGATCACCTTGGGGTACACCCAGG
CCGACGTGGGGCTCACCCTGGGCGTTCTCTTTGGAAAGGT
GTTCAGCCAGACCACCATCTGTCGCTTCGAGGCCTTGCAG
CTCAGCCTTAAGAACATGTGTAAGCTGCGGCCCCTGCTGG
AGAAGTGGGTGGAGGAAGCCGACAACAATGAGAACCTTCA
GGAGATATGCAAATCGGAGACCCTGGTGCAGGCCCGGAAG
AGAAAGCGAACTAGCATTGAGAACCGTGTGAGGTGGAGTC
TGGAGACCATGTTTCTGAAGTGCCCGAAGCCCTCCCTACA
GCAGATCACTCACATCGCCAATCAGCTTGGGCTAGAGAAG
GATGTGGTTCGAGTATGGTTCTGTAACCGGCGCCAGAAGG
GCAAAAGATCAAGTATTGAGTATTCCCAACGAGAAGAGTA
TGAGGCTACAGGGACACCTTTCCCAGGGGGGGCTGTATCC
TTTCCTCTGCCCCCAGGTCCCCACTTTGGCACCCCAGGCT
ATGGAAGCCCCCACTTCACCACACTCTACTCAGTCCCTTT
TCCTGAGGGCGAGGCCTTTCCCTCTGTTCCCGTCACTGCT
CTGGGCTCTCCCATGCATTCAAACGCTAGCGGCAGCGGCG
CCACGAACTTCTCTCTGTTAAAGCAAGCAGGAGATGTTGA
AGAAAACCCCGGGCCTGCATGCATGTATAACATGATGGAG
ACGGAGCTGAAGCCGCCGGGCCCGCAGCAAGCTTCGGGGG
GCGGCGGCGGAGGAGGCAACGCCACGGCGGCGGCGACCGG
CGGCAACCAGAAGAACAGCCCGGACCGCGTCAAGAGGCCC
ATGAACGCCTTCATGGTATGGTCCCGGGGGCAGCGGCGTA
AGATGGCCCAGGAGAACCCCAAGATGCACAACTCGGAGAT
CAGCAAGCGCCTGGGCGCGGAGTGGAAACTTTTGTCCGAG
ACCGAGAAGCGGCCGTTCATCGACGAGGCCAAGCGGCTGC
GCGCTCTGCACATGAAGGAGCACCCGGATTATAAATACCG
GCCGCGGCGGAAAACCAAGACGCTCATGAAGAAGGATAAG
TACACGCTTCCCGGAGGCTTGCTGGCCCCCGGCGGGAACA
GCATGGCGAGCGGGGTTGGGGTGGGCGCCGGCCTGGGTGC
GGGCGTGAACCAGCGCATGGACAGCTACGCGCACATGAAC
GGCTGGAGCAACGGCAGCTACAGCATGATGCAGGAGCAGC
TGGGCTACCCGCAGCACCCGGGCCTCAACGCTCACGGCGC
GGCACAGATGCAACCGATGCACCGCTACGACGTCAGCGCC
CTGCAGTACAACTCCATGACCAGCTCGCAGACCTACATGA
ACGGCTCGCCCACCTACAGCATGTCCTACTCGCAGCAGGG
CACCCCCGGTATGGCGCTGGGCTCCATGGGCTCTGTGGTC
AAGTCCGAGGCCAGCTCCAGCCCCCCCGTGGTTACCTCTT
CCTCCCACTCCAGGGCGCCCTGCCAGGCCGGGGACCTCCG
GGACATGATCAGCATGTACCTCCCCGGCGCCGAGGTGCCG
GAGCCCGCTGCGCCCAGTAGACTGCACATGGCCCAGCACT
ACCAGAGCGGCCCGGTGCCCGGCACGGCCATTAACGGCAC
ACTGCCCCTGTCGCACATGGCATGCGGCTCCGGCGAGGGC
AGGGGAAGTCTTCTAACATGCGGGGACGTGGAGGAAAATC
CCGGCCCACTCGAGATGAGGCAGCCACCTGGCGAGTCTGA
CATGGCTGTCAGCGACGCTCTGCTCCCGTCCTTCTCCACG
TTCGCGTCCGGCCCGGCGGGAAGGGAGAAGACACTGCGTC
CAGCAGGTGCCCCGACTAACCGTTGGCGTGAGGAACTCTC
TCACATGAAGCGACTTCCCCCACTTCCCGGCCGCCCCTAC
GACCTGGCGGCGACGGTGGCCACAGACCTGGAGAGTGGCG
GAGCTGGTGCAGCTTGCAGCAGTAACAACCCGGCCCTCCT
AGCCCGGAGGGAGACCGAGGAGTTCAACGACCTCCTGGAC
CTAGACTTTATCCTTTCCAACTCGCTAACCCACCAGGAAT
CGGTGGCCGCCACCGTGACCACCTCGGCGTCAGCTTCATC
CTCGTCTTCCCCAGCGAGCAGCGGCCCTGCCAGCGCGCCC
TCCACCTGCAGCTTCAGCTATCCGATCCGGGCCGGGGGTG
ACCCGGGCGTGGCTGCCAGCAACACAGGTGGAGGGCTCCT
CTACAGCCGAGAATCTGCGCCACCTCCCACGGCCCCCTTC
AACCTGGCGGACATCAATGACGTGAGCCCCTCGGGCGGCT
TCGTGGCTGAGCTCCTGCGGCCGGAGTTGGACCCAGTATA
CATTCCGCCACAGCAGCCTCAGCCGCCAGGTGGCGGGCTG
ATGGGCAAGTTTGTGCTGAAGGCGTCTCTGACCACCCCTG
GCAGCGAGTACAGCAGCCCTTCGGTCATCAGTGTTAGCAA
AGGAAGCCCAGACGGCAGCCACCCCGTGGTAGTGGCGCCC
TACAGCGGTGGCCCGCCGCGCATGTGCCCCAAGATTAAGC
AAGAGGCGGTCCCGTCCTGCACGGTCAGCCGGTCCCTAGA
GGCCCATTTGAGCGCTGGACCCCAGCTCAGCAACGGCCAC
CGGCCCAACACACACGACTTCCCCCTGGGGCGGCAGCTCC
CCACCAGGACTACCCCTACACTGAGTCCCGAGGAACTGCT
GAACAGCAGGGACTGTCACCCTGGCCTGCCTCTTCCCCCA
GGATTCCATCCCCATCCGGGGCCCAACTACCCTCCTTTCC
TGCCAGACCAGATGCAGTCACAAGTCCCCTCTCTCCATTA
TCAAGAGCTCATGCCACCGGGTTCCTGCCTGCCAGAGGAG
CCCAAGCCAAAGAGGGGAAGAAGGTCGTGGCCCCGGAAAA
GAACAGCCACCCACACTTGTGACTATGCAGGCTGTGGCAA
AACCTATACCAAGAGTTCTCATCTCAAGGCACACCTGCGA
ACTCACACAGGCGAGAAACCTTACCACTGTGACTGGGACG
GCTGTGGGTGGAAATTCGCCCGCTCCGATGAACTGACCAG
GCACTACCGCAAACACACAGGGCACCGGCCCTTTCAGTGC
CAGAAGTGCGACAGGGCCTTTTCCAGGTCGGACCACCTTG
CCTTACACATGAAGAGGCACTAAATGACTAGTGCGCGCAG
CGGCCGACCATGGCCCAACTTGTTTATTGCAGCTTATAAT
GGTTACAAATAAAGCAATAGCATCACAAATTTCACAAATA
AAGCATTTTTTTCACTGCATTCTAGTTGTGGTTTGTCCAA
ACTCATCAATGTATCTTATCATGTCTGGATCTCGGTACCG
GATCCAAATTCCCGATAAGGATCTTCCTAGAGCATGGCTA
CGTAGATAAGTAGCATGGCGGGTTAATCATTAACTACAAG
GAACCCCTAGTGATGGAGTTGGCCACTCCCTCTCTGCGCG
CTCGCTCGCTCACTGAGGCCGGGCGACCAAAGGTCGCCCG
ACGCCCGGGCTTTGCCCGGGCGGCCTCAGTGAGCGAGCGA
GCGCGCAGCCTTAATTAACCTAATTCACTGGCCGTCGTTT
TACAACGTCGTGACTGGGAAAACCCTGGCGTTACCCAACT
TAATCGCCTTGCAGCACATCCCCCTTTCGCCAGCTGGCGT
AATAGCGAAGAGGCCCGCACCGATCGCCCTTCCCAACAGT
TGCGCAGCCTGAATGGCGAATGGGACGCGCCCTGTAGCGG
CGCATTAAGCGCGGCGGGTGTGGTGGTTACGCGCAGCGTG
ACCGCTACACTTGCCAGCGCCCTAGCGCCCGCTCCTTTCG
CTTTCTTCCCTTCCTTTCTCGCCACGTTCGCCGGCTTTCC
CCGTCAAGCTCTAAATCGGGGGCTCCCTTTAGGGTTCCGA
TTTAGTGCTTTACGGCACCTCGACCCCAAAAAACTTGATT
AGGGTGATGGTTCACGTAGTGGGCCATCGCCCTGATAGAC
GGTTTTTCGCCCTTTGACGTTGGAGTCCACGTTCTTTAAT
AGTGGACTCTTGTTCCAAACTGGAACAACACTCAACCCTA
TCTCGGTCTATTCTTTTGATTTATAAGGGATTTTGCCGAT
TTCGGCCTATTGGTTAAAAAATGAGCTGATTTAACAAAAA
TTTAACGCGAATTTTAACAAAATATTAACGTTTATAATTT
CAGGTGGCATCTTTCGGGGAAATGTGCGCGGAACCCCTAT
TTGTTTATTTTTCTAAATACATTCAAATATGTATCCGCTC
ATGAGACAATAACCCTGATAAATGCTTCAATAATATTGAA
AAAGGAAGAGTATGAGTATTCAACATTTCCGTGTCGCCCT
TATTCCCTTTTTTGCGGCATTTTGCCTTCCTGTTTTTGCT
CACCCAGAAACGCTGGTGAAAGTAAAAGATGCTGAAGATC
AGTTGGGTGCACGAGTGGGTTACATCGAACTGGATCTCAA
TAGTGGTAAGATCCTTGAGAGTTTTCGCCCCGAAGAACGT
TTTCCAATGATGAGCACTTTTAAAGTTCTGCTATGTGGCG
CGGTATTATCCCGTATTGACGCCGGGCAAGAGCAACTCGG
TCGCCGCATACACTATTCTCAGAATGACTTGGTTGAGTAC
TCACCAGTCACAGAAAAGCATCTTACGGATGGCATGACAG
TAAGAGAA
Nucleic acid sequence of pAAV-UBC-rtTA4-
WPRE3-SV40pA vector (SEQ ID NO: 17):
TTATGCAGTGCTGCCATAACCATGAGTGATAACACTGCGG
CCAACTTACTTCTGACAACGATCGGAGGACCGAAGGAGCT
AACCGCTTTTTTGCACAACATGGGGGATCATGTAACTCGC
CTTGATCGTTGGGAACCGGAGCTGAATGAAGCCATACCAA
ACGACGAGCGTGACACCACGATGCCTGTAGTAATGGTAAC
AACGTTGCGCAAACTATTAACTGGCGAACTACTTACTCTA
GCTTCCCGGCAACAATTAATAGACTGGATGGAGGCGGATA
AAGTTGCAGGACCACTTCTGCGCTCGGCCCTTCCGGCTGG
CTGGTTTATTGCTGATAAATCTGGAGCCGGTGAGCGTGGG
TCTCGCGGTATCATTGCAGCACTGGGGCCAGATGGTAAGC
CCTCCCGTATCGTAGTTATCTACACGACGGGGAGTCAGGC
AACTATGGATGAACGAAATAGACAGATCGCTGAGATAGGT
GCCTCACTGATTAAGCATTGGTAACTGTCAGACCAAGTTT
ACTCATATATACTTTAGATTGATTTAAAACTTCATTTTTA
ATTTAAAAGGATCTAGGTGAAGATCCTTTTTGATAATCTC
ATGACCAAAATCCCTTAACGTGAGTTTTCGTTCCACTGAG
CGTCAGACCCCGTAGAAAAGATCAAAGGATCTTCTTGAGA
TCCTTTTTTTCTGCGCGTAATCTGCTGCTTGCAAACAAAA
AAACCACCGCTACCAGCGGTGGTTTGTTTGCCGGATCAAG
AGCTACCAACTCTTTTTCCGAAGGTAACTGGCTTCAGCAG
AGCGCAGATACCAAATACTGTCCTTCTAGTGTAGCCGTAG
TTAGGCCACCACTTCAAGAACTCTGTAGCACCGCCTACAT
ACCTCGCTCTGCTAATCCTGTTACCAGTGGCTGCTGCCAG
TGGCGATAAGTCGTGTCTTACCGGGTTGGACTCAAGACGA
TAGTTACCGGATAAGGCGCAGCGGTCGGGCTGAACGGGGG
GTTCGTGCACACAGCCCAGCTTGGAGCGAACGACCTACAC
CGAACTGAGATACCTACAGCGTGAGCTATGAGAAAGCGCC
ACGCTTCCCGAAGGGAGAAAGGCGGACAGGTATCCGGTAA
GCGGCAGGGTCGGAACAGGAGAGCGCACGAGGGAGCTTCC
AGGGGGAAACGCCTGGTATCTTTATAGTCCTGTCGGGTTT
CGCCACCTCTGACTTGAGCGTCGATTTTTGTGATGCTCGT
CAGGGGGGCGGAGCCTATGGAAAAACGCCAGCAACGCGGC
CTTTTTACGGTTCCTGGCCTTTTGCTGGCCTTTTGCTCAC
ATGTTCTTTCCTGCGTTATCCCCTGATTCTGTGGATAACC
GTATTACCGCCTTTGAGTGAGCTGATACCGCTCGCCGCAG
CCGAACGACCGAGCGCAGCGAGTCAGTGAGCGAGGAAGCG
GAAGAGCGCCCAATACGCAAACCGCCTCTCCCCGCGCGTT
GGCCGATTCATTAATGCAGCTGGCACGACAGGTTTCCCGA
CTGGAAAGCGGGCAGTGAGCGCAACGCAATTAATGTGAGT
TAGCTCACTCATTAGGCACCCCAGGCTTTACACTTTATGC
TTCCGGCTCGTATGTTGTGTGGAATTGTGAGCGGATAACA
ATTTCACACAGGAAACAGCTATGACCATGATTACGCCAGA
TTTAATTAAGGCCTTAATTAGGCTGCGCGCTCGCTCGCTC
ACTGAGGCCGCCCGGGCAAAGCCCGGGCGTCGGGCGACCT
TTGGTCGCCCGGCCTCAGTGAGCGAGCGAGCGCGCAGAGA
GGGAGTGGCCAACTCCATCACTAGGGGTTCCTTGTAGTTA
ATGATTAACCCGCCATGCTACTTATCTACGTAGCCATGCT
CTAGGAAGATCGGAATTCCTGATCTGGCCTCCGCGCCGGG
TTTTGGCGCCTCCCGCGGGCGCCCCCCTCCTCACGGCGAG
CGCTGCCACGTCAGACGAAGGGCGCAGCGAGCGTCCTGAT
CCTTCCGCCCGGACGCTCAGGACAGCGGCCCGCTGCTCAT
AAGACTCGGCCTTAGAACCCCAGTATCAGCAGAAGGACAT
TTTAGGACGGGACTTGGGTGACTCTAGGGCACTGGTTTTC
TTTCCAGAGAGCGGAACAGGCGAGGAAAAGTAGTCCCTTC
TCGGCGATTCTGCGGAGGGATCTCCGTGGGGCGGTGAACG
CCGATGATTATATAAGGACGCGCCGGGTGTGGCACAGCTA
GTTCCGTCGCAGCCGGGATTTGGGTCGCGGTTCTTGTTTG
TGGATCGCTGTGATCGTCACTTGGTGAGTAGCGGGCTGCT
GGGCTGGCCGGGGCTTTCGTGGCCGCCGGGCCGCTCGGTG
GGACGGAAGCGTGTGGAGAGACCGCCAAGGGCTGTAGTCT
GGGTCCGCGAGCAAGGTTGCCCTGAACTGGGGGTTGGGGG
GAGCGCAGCAAAATGGCGGCTGTTCCCGAGTCTTGAATGG
AAGACGCTTGTGAGGCGGGCTGTGAGGTCGTTGAAACAAG
GTGGGGGGCATGGTGGGCGGCAAGAACCCAAGGTCTTGAG
GCCTTCGCTAATGCGGGAAAGCTCTTATTCGGGTGAGATG
GGCTGGGGCACCATCTGGGGACCCTGACGTGAAGTTTGTC
ACTGACTGGAGAACTCGGTTTGTCGTCTGTTGCGGGGGCG
GCAGTTATGCGGTGCCGTTGGGCAGTGCACCCGTACCTTT
GGGAGCGCGCGCCTCGTCGTGTCGTGACGTCACCCGTTCT
GTTGGCTTATAATGCAGGGTGGGGCCACCTGCCGGTAGGT
GTGCGGTAGGCTTTTCTCCGTCGCAGGACGCAGGGTTCGG
GCCTAGGGTAGGCTCTCCTGAATCGACAGGCGCCGGACCT
CTGGTGAGGGGAGGGATAAGTGAGGCGTCAGTTTCTTTGG
TCGGTTTTATGTACCTATCTTCTTAAGTAGCTGAAGCTCC
GGTTTTGAACTATGCGCTCGGGGTTGGCGAGTGTGTTTTG
TGAAGTTTTTTAGGCACCTTTTGAAATGTAATCATTTGGG
TCAATATGTAATTTTCAGTGTTAGACTAGTAAATTGTCCG
CTAAATTCTGGCCGTTTTTGGCTTTTTTGTTAGACGAAGC
GGCCGCATTAAACGCCACCATGTCCCGCTTGGATAAGAGC
AAGGTAATAAATAGCGCACTCGAACTCCTCAACGGCGTGG
GCATCGAAGGTCTGACTACTCGAAAGCTCGCCCAGAAATT
GGGTGTGGAGCAACCTACATTGTATTGGCATGTCAAGAAC
AAAAGAGCCCTGCTGGACGCTCTTCCTATTGAAATGCTTG
ACAGGCATCACACTCATTCCTGCCCCCTTGAGGTCGAGAG
TTGGCAAGATTTTCTCCGAAACAATGCAAAGTCCTACCGC
TGCGCACTTTTGTCCCATAGGGATGGAGCAAAAGTGCACC
TGGGAACCAGGCCAACAGAGAAACAATACGAGACTCTCGA
GAACCAGTTGGCTTTCTTGTGCCAACAGGGGTTCTCACTT
GAAAATGCCCTTTACGCACTGTCAGCCGTTGGACATTTTA
CCCTGGGGTGCGTTCTTGAGGAGCAAGAACATCAGGTTGC
TAAGGAGGAGCGCGAGACTCCAACCACTGATTCTATGCCA
CCTTTGCTGAAACAGGCCATTGAACTTTTCGATAGACAGG
GTGCTGAACCTGCCTTTCTCTTCGGGTTGGAGCTGATTAT
TTGTGGTCTCGAAAAACAGCTGAAATGTGAAAGTGGTGGC
CCTACTGACGCCCTCGATGATTTCGACCTGGATATGCTGC
CAGCCGATGCACTTGATGATTTCGATTTGGATATGCTTCC
AGCCGACGCACTGGACGACTTCGATTTGGACATGCTTCCC
GGTTAAACTAGTCTAGCAATCAACCTCTGGATTACAAAAT
TTGTGAAAGATTGACTGGTATTCTTAACTATGTTGCTCCT
TTTACGCTATGTGGATACGCTGCTTTAATGCCTTTGTATC
ATGCTATTGCTTCCCGTATGGCTTTCATTTTCTCCTCCTT
GTATAAATCCTGGTTAGTTCTTGCCACGGCGGAACTCATC
GCCGCCTGCCTTGCCCGCTGCTGGACAGGGGCTCGGCTGT
TGGGCACTGACAATTCCGTGGTGTTTATTTGTGAAATTTG
TGATGCTATTGCTTTATTTGTAACCATTCTAGCTTTATTT
GTGAAATTTGTGATGCTATTGCTTTATTTGTAACCATTAT
AAGCTGCAATAAACAAGTTAACAACAACAATTGCATTCAT
TTTATGTTTCAGGTTCAGGGGGAGATGTGGGAGGTTTTTT
AAAGCGGGGGATCCAAATTCCCGATAAGGATCTTCCTAGA
GCATGGCTACGTAGATAAGTAGCATGGCGGGTTAATCATT
AACTACAAGGAACCCCTAGTGATGGAGTTGGCCACTCCCT
CTCTGCGCGCTCGCTCGCTCACTGAGGCCGGGCGACCAAA
GGTCGCCCGACGCCCGGGCTTTGCCCGGGCGGCCTCAGTG
AGCGAGCGAGCGCGCAGCCTTAATTAACCTAATTCACTGG
CCGTCGTTTTACAACGTCGTGACTGGGAAAACCCTGGCGT
TACCCAACTTAATCGCCTTGCAGCACATCCCCCTTTCGCC
AGCTGGCGTAATAGCGAAGAGGCCCGCACCGATCGCCCTT
CCCAACAGTTGCGCAGCCTGAATGGCGAATGGGACGCGCC
CTGTAGCGGCGCATTAAGCGCGGCGGGTGTGGTGGTTACG
CGCAGCGTGACCGCTACACTTGCCAGCGCCCTAGCGCCCG
CTCCTTTCGCTTTCTTCCCTTCCTTTCTCGCCACGTTCGC
CGGCTTTCCCCGTCAAGCTCTAAATCGGGGGCTCCCTTTA
GGGTTCCGATTTAGTGCTTTACGGCACCTCGACCCCAAAA
AACTTGATTAGGGTGATGGTTCACGTAGTGGGCCATCGCC
CTGATAGACGGTTTTTCGCCCTTTGACGTTGGAGTCCACG
TTCTTTAATAGTGGACTCTTGTTCCAAACTGGAACAACAC
TCAACCCTATCTCGGTCTATTCTTTTGATTTATAAGGGAT
TTTGCCGATTTCGGCCTATTGGTTAAAAAATGAGCTGATT
TAACAAAAATTTAACGCGAATTTTAACAAAATATTAACGT
TTATAATTTCAGGTGGCATCTTTCGGGGAAATGTGCGCGG
AACCCCTATTTGTTTATTTTTCTAAATACATTCAAATATG
TATCCGCTCATGAGACAATAACCCTGATAAATGCTTCAAT
AATATTGAAAAAGGAAGAGTATGAGTATTCAACATTTCCG
TGTCGCCCTTATTCCCTTTTTTGCGGCATTTTGCCTTCCT
GTTTTTGCTCACCCAGAAACGCTGGTGAAAGTAAAAGATG
CTGAAGATCAGTTGGGTGCACGAGTGGGTTACATCGAACT
GGATCTCAATAGTGGTAAGATCCTTGAGAGTTTTCGCCCC
GAAGAACGTTTTCCAATGATGAGCACTTTTAAAGTTCTGC
TATGTGGCGCGGTATTATCCCGTATTGACGCCGGGCAAGA
GCAACTCGGTCGCCGCATACACTATTCTCAGAATGACTTG
GTTGAGTACTCACCAGTCACAGAAAAGCATCTTACGGATG
GCATGACAGTAAGAGAA
UBC promoter sequence (SEQ ID NO: 18):
GATCTGGCCTCCGCGCCGGGTTTTGGCGCCTCCCGCGGGC
GCCCCCCTCCTCACGGCGAGCGCTGCCACGTCAGACGAAG
GGCGCAGCGAGCGTCCTGATCCTTCCGCCCGGACGCTCAG
GACAGCGGCCCGCTGCTCATAAGACTCGGCCTTAGAACCC
CAGTATCAGCAGAAGGACATTTTAGGACGGGACTTGGGTG
ACTCTAGGGCACTGGTTTTCTTTCCAGAGAGCGGAACAGG
CGAGGAAAAGTAGTCCCTTCTCGGCGATTCTGCGGAGGGA
TCTCCGTGGGGCGGTGAACGCCGATGATTATATAAGGACG
CGCCGGGTGTGGCACAGCTAGTTCCGTCGCAGCCGGGATT
TGGGTCGCGGTTCTTGTTTGTGGATCGCTGTGATCGTCAC
TTGGTGAGTAGCGGGCTGCTGGGCTGGCCGGGGCTTTCGT
GGCCGCCGGGCCGCTCGGTGGGACGGAAGCGTGTGGAGAG
ACCGCCAAGGGCTGTAGTCTGGGTCCGCGAGCAAGGTTGC
CCTGAACTGGGGGTTGGGGGGAGCGCAGCAAAATGGCGGC
TGTTCCCGAGTCTTGAATGGAAGACGCTTGTGAGGCGGGC
TGTGAGGTCGTTGAAACAAGGTGGGGGGCATGGTGGGCGG
CAAGAACCCAAGGTCTTGAGGCCTTCGCTAATGCGGGAAA
GCTCTTATTCGGGTGAGATGGGCTGGGGCACCATCTGGGG
ACCCTGACGTGAAGTTTGTCACTGACTGGAGAACTCGGTT
TGTCGTCTGTTGCGGGGGCGGCAGTTATGCGGTGCCGTTG
GGCAGTGCACCCGTACCTTTGGGAGCGCGCGCCTCGTCGT
GTCGTGACGTCACCCGTTCTGTTGGCTTATAATGCAGGGT
GGGGCCACCTGCCGGTAGGTGTGCGGTAGGCTTTTCTCCG
TCGCAGGACGCAGGGTTCGGGCCTAGGGTAGGCTCTCCTG
AATCGACAGGCGCCGGACCTCTGGTGAGGGGAGGGATAAG
TGAGGCGTCAGTTTCTTTGGTCGGTTTTATGTACCTATCT
TCTTAAGTAGCTGAAGCTCCGGTTTTGAACTATGCGCTCG
GGGTTGGCGAGTGTGTTTTGTGAAGTTTTTTAGGCACCTT
TTGAAATGTAATCATTTGGGTCAATATGTAATTTTCAGTG
TTAGACTAGTAAATTGTCCGCTAAATTCTGGCCGTTTTTG
GCTTTTTTGTTAGAC
Tet-O sequence (SEQ ID NO: 19):
TCCCTATCAGTGATAGAGA
Nucleic acid sequence encoding minimal
CMV promoter (SEQ ID NO: 20):
GCTTTAGGCGTGTACGGTGGGCGCCTATAAAAGCAGAGCT
CGTTTAGTGAACCGTCAGATCGCCTGGA
Nucleic acid sequence encoding WPRE
(SEQ ID NO: 21):
AATCAACCTCTGGATTACAAAATTTGTGAAAGATTGACTG
GTATTCTTAACTATGTTGCTCCTTTTACGCTATGTGGATA
CGCTGCTTTAATGCCTTTGTATCATGCTATTGCTTCCCGT
ATGGCTTTCATTTTCTCCTCCTTGTATAAATCCTGGTTAG
TTCTTGCCACGGCGGAACTCATCGCCGCCTGCCTTGCCCG
CTGCTGGACAGGGGCTCGGCTGTTGGGCACTGACAATTCC
GTGGTGTT
Nucleic acid sequence encoding
inverted terminal
repeat sequence (SEQ ID NO: 22):
CCTTAATTAGGCTGCGCGCTCGCTCGCTCACTGAGGCCGC
CCGGGCAAAGCCCGGGCGTCGGGCGACCTTTGGTCGCCCG
GCCTCAGTGAGCGAGCGAGCGCGCAGAGAGGGAGTGGCCA
ACTCCATCACTAGGGGTTCCT
Nucleic acid sequence of a TRE2 promoter
(a non-limiting example of a TRE promoter)
(SEQ ID NO: 23):
AATTCGTACACGCCTACCTCGACCCATCAAGTGCCACCTG
ACGTCTCCCTATCAGTGATAGAGAAGTCGACACGTCTCGA
GCTCCCTATCAGTGATAGAGAAGGTACGTCTAGAACGTCT
CCCTATCAGTGATAGAGAAGTCGACACGTCTCGAGCTCCC
TATCAGTGATAGAGAAGGTACGTCTAGAACGTCTCCCTAT
CAGTGATAGAGAAGTCGACACGTCTCGAGCTCCCTATCAG
TGATAGAGAAGGTACGTCTAGAACGTCTCCCTATCAGTGA
TAGAGAAGTCGACACGTCTCGAGCTCCCTATCAGTGATAG
AGAAGGTACCCCCTATATAAGCAGAGCTCGTTTAGTGAAC
CGTCAGATCGCCTGGAGACGCCATCCACGCTGTTTTGAC
CTCCATAGAAGACACCGGGACCGATCCAGCCTGGATCGC
Nucleic acid sequence of P tight promoter
(a non-limiting example of a TRE promoter)
(SEQ ID NO: 24):
GAGTTTACTCCCTATCAGTGATAGAGAACGTATGTCGAGT
TTACTCCCTATCAGTGATAGAGAACGATGTCGAGTTTACT
CCCTATCAGTGATAGAGAACGTATGTCGAGTTTACTCCCT
ATCAGTGATAGAGAACGTATGTCGAGTTTACTCCCTATCA
GTGATAGAGAACGTATGTCGAGTTTATCCCTATCAGTGAT
AGAGAACGTATGTCGAGTTTACTCCCTATCAGTGATAGAG
AACGTATGTCGAGGTAGGCGTGTACGGTGGGAGGCCTATA
TAAGCAGAGCTCGTTTAGTGAACCGTCAGATCGCC
Nucleic acid sequence encoding TetR
(SEQ ID NO: 25):
ATGGCTAGATTAGATAAAAGTAAAGTGATTAACAGCGCAT
TAGAGCTGCTTAATGAGGTCGGAATCGAAGGTTTAACAAC
CCGTAAACTCGCCCAGAAGCTAGGTGTAGAGCAGCCTACA
TTGTATTGGCATGTAAAAAATAAGCGGGCTTTGCTCGACG
CCTTAGCCATTGAGATGTTAGATAGGCACCATACTCACTT
TTGCCCTTTAGAAGGGGAAAGCTGGCAAGATTTTTTACGT
AATAACGCTAAAAGTTTTAGATGTGCTTTACTAAGTCATC
GCGATGGAGCAAAAGTACATTTAGGTACACGGCCTACAGA
AAAACAGTATGAAACTCTCGAAAATCAATTAGCCTTTTTA
TGCCAACAAGGTTTTTCACTAGAGAATGCATTATATGCA
CTCAGCGCTGTGGGGCATTTTACTTTAGGTTGCGTATTGG
AAGATCAAGAGCATCAAGTCGCTAAAGAAGAAAGGGAAAC
ACCTACTACTGATAGTATGCCGCCATTATTACGACAAGCT
ATCGAATTATTTGATCACCAAGGTGCAGAGCCAGCCTTCT
TATTCGGCCTTGAATTGATCATATGCGGATTAGAAAAACA
ACTTAAATGTGAAAGTGGG
Amino acid sequence encoding TetR
(SEQ ID NO: 26):
MARLDKSKVINSALELLNEVGIEGLTTRKLAQKLGVEQPT
LYWHVKNKRALLDALAIEMLDRHHTHFCPLEGESWQDFLR
NNAKSFRCALLSHRDGAKVHLGTRPTEKQYETLENQLAFL
CQQGFSLENALYALSAVGHFTLGCVLEDQEHQVAKEERET
PTTDSMPPLLRQAIELFDHQGAEPAFLFGLELIICGLEKQ
LKCESG
Nucleic acid sequence encoding
TetR-Krab (SEQ ID NO: 27)
ATGGCTAGATTAGATAAAAGTAAAGTGATTAACAGCGCAT
TAGAGCTGCTTAATGAGGTCGGAATCGAAGGTTTAACAAC
CCGTAAACTCGCCCAGAAGCTAGGTGTAGAGCAGCCTACA
TTGTATTGGCATGTAAAAAATAAGCGGGCTTTGCTCGACG
CCTTAGCCATTGAGATGTTAGATAGGCACCATACTCACTT
TTGCCCTTTAGAAGGGGAAAGCTGGCAAGATTTTTTACGT
AATAACGCTAAAAGTTTTAGATGTGCTTTACTAAGTCATC
GCGATGGAGCAAAAGTACATTTAGGTACACGGCCTACAGA
AAAACAGTATGAAACTCTCGAAAATCAATTAGCCTTTTTA
TGCCAACAAGGTTTTTCACTAGAGAATGCATTATATGCAC
TCAGCGCTGTGGGGCATTTTACTTTAGGTTGCGTATTGGA
AGATCAAGAGCATCAAGTCGCTAAAGAAGAAAGGGAAACA
CCTACTACTGATAGTATGCCGCCATTATTACGACAAGCTA
TCGAATTATTTGATCACCAAGGTGCAGAGCCAGCCTTCTT
ATTCGGCCTTGAATTGATCATATGCGGATTAGAAAAACAA
CTTAAATGTGAAAGTGGGTCGCCAAAAAAGAAGAGAAAGG
TCGACGGCGGTGGTGCTTTGTCTCCTCAGCACTCTGCTGT
CACTCAAGGAAGTATCATCAAGAACAAGGAGGGCATGGAT
GCTAAGTCACTAACTGCCTGGTCCCGGACACTGGTGACCT
TCAAGGATGTATTTGTGGACTTCACCAGGGAGGAGTGGAA
GCTGCTGGACACTGCTCAGCAGATCGTGTACAGAAATGTG
ATGCTGGAGAACTATAAGAACCTGGTTTCCTTGGGTTATC
AGCTTACTAAGCCAGATGTGATCCTCCGGTTGGAGAAGGG
AGAAGAGCCCTGGCTGGTGGAGAGAGAAATTCACCAAGAG
ACCCATCCTGATTCAGAGACTGCATTTGAAATCAAATCAT
CAGTTTAA
Amino acid sequence encoding
TetR-KRAB (SEQ ID NO: 28):
MARLDKSKVINSALELLNEVGIEGLTTRKLAQKLGVEQPT
LYWHVKNKRALLDALAIEMLDRHHTHFCPLEGESWQDFLR
NNAKSFRCALLSHRDGAKVHLGTRPTEKQYETLENQLAFL
CQQGFSLENALYALSAVGHFTLGCVLEDQEHQVAKEERET
PTTDSMPPLLRQAIELFDHQGAEPAFLFGLELIICGLEKQ
LKCESGSPKKKRKVDGGGALSPQHSAVTQGSIIKNKEGMD
AKSLTAWSRTLVTFKDVFVDFTREEWKLLDTAQQIVYRNV
MLENYKNLVSLGYQLTKPDVILRLEKGEEPWLVEREIHQE
THPDSETAFEIKSSV
Desmin promoter (SEQ ID NO: 29):
ACCTTGCTTCCTAGCTGGGCCTTTCCTTCTCCTCTATAAA
TACCAGCTCTGGTATTTCGCCTTGGCAGCTGTTGCTGCTA
GGGAGACGGCTGGCTTGACATGCATCTCCTGACAAAACAC
AAACCCGTGGTGTGAGTGGGTGTGGGCGGTGTGAGTAGGG
GGATGAATCAGAGAGGGGGCGAGGGAGACAGGGGCGCAGG
AGTCAGGCAAAGGCGATGCGGGGGTGCGACTACACGCAGT
TGGAAACAGTCGTCAGAAGATTCTGGAAACTATCTTGCTG
GCTATAAACTTGAGGGAAGCAGAAGGCCAACATTCCTCCC
AAGGGAAACTGAGGCTCAGAGTTAAAACCCAGGTATCAGT
GATATGCATGTGCCCCGGCCAGGGTCACTCTCTGACTAAC
CGGTACCTACCCTACAGGCCTACCTAGAGACTCTTTTGAA
AGGATGGTAGAGACCTGTCCGGGCTTTGCCCACAGTCGTT
GGAAACCTCAGCATTTTCTAGGCAACTTGTGCGAATAAAA
CACTTCGGGGGTCCTTCTTGTTCATTCCAATAACCTAAAA
CCTCTCCTCGGAGAAAATAGGGGGCCTCAAACAAACGAAA
TTCTCTAGCCCGCTTTCCCCAGGATAAGGCAGGCATCCAA
ATGGAAAAAAAGGGGCCGGCCGGGGGTCTCCTGTCAGCTC
CTTGCCCTGTGAAACCCAGCAGGCCTGCCTGTCTTCTGTC
CTCTTGGGGCTGTCCAGGGGCGCAGGCCTCTTGCGGGGGA
GCTGGCCTCCCCGCCCCCTCGCCTGTGGCCGCCCTTTTCC
TGGCAGGACAGAGGGATCCTGCAGCTGTCAGGGGAGGGGC
GCCGGGGGGTGATGTCAGGAGGGCTACAAATAGTGCAGAC
AGCTAAGGGGCTCCGTCACCCATCTTCACATCCACTCCAG
CCGGCTGCCCGCCCGCTGCCTCCTCTGTGCGTCCGCCCAG
CCAGCCTCGTCCACGCC
Desmin-rtTA4 vector (SEQ ID NO: 30):
TTATGCAGTGCTGCCATAACCATGAGTGATAACACTGCGG
CCAACTTACTTCTGACAACGATCGGAGGACCGAAGGAGCT
AACCGCTTTTTTGCACAACATGGGGGATCATGTAACTCGC
CTTGATCGTTGGGAACCGGAGCTGAATGAAGCCATACCAA
ACGACGAGCGTGACACCACGATGCCTGTAGTAATGGTAAC
AACGTTGCGCAAACTATTAACTGGCGAACTACTTACTCTA
GCTTCCCGGCAACAATTAATAGACTGGATGGAGGCGGATA
AAGTTGCAGGACCACTTCTGCGCTCGGCCCTTCCGGCTGG
CTGGTTTATTGCTGATAAATCTGGAGCCGGTGAGCGTGGG
TCTCGCGGTATCATTGCAGCACTGGGGCCAGATGGTAAGC
CCTCCCGTATCGTAGTTATCTACACGACGGGGAGTCAGGC
AACTATGGATGAACGAAATAGACAGATCGCTGAGATAGGT
GCCTCACTGATTAAGCATTGGTAACTGTCAGACCAAGTTT
ACTCATATATACTTTAGATTGATTTAAAACTTCATTTTTA
ATTTAAAAGGATCTAGGTGAAGATCCTTTTTGATAATCTC
ATGACCAAAATCCCTTAACGTGAGTTTTCGTTCCACTGAG
CGTCAGACCCCGTAGAAAAGATCAAAGGATCTTCTTGAGA
TCCTTTTTTTCTGCGCGTAATCTGCTGCTTGCAAACAAAA
AAACCACCGCTACCAGCGGTGGTTTGTTTGCCGGATCAAG
AGCTACCAACTCTTTTTCCGAAGGTAACTGGCTTCAGCAG
AGCGCAGATACCAAATACTGTCCTTCTAGTGTAGCCGTAG
TTAGGCCACCACTTCAAGAACTCTGTAGCACCGCCTACAT
ACCTCGCTCTGCTAATCCTGTTACCAGTGGCTGCTGCCAG
TGGCGATAAGTCGTGTCTTACCGGGTTGGACTCAAGACGA
TAGTTACCGGATAAGGCGCAGCGGTCGGGCTGAACGGGGG
GTTCGTGCACACAGCCCAGCTTGGAGCGAACGACCTACAC
CGAACTGAGATACCTACAGCGTGAGCTATGAGAAAGCGCC
ACGCTTCCCGAAGGGAGAAAGGCGGACAGGTATCCGGTAA
GCGGCAGGGTCGGAACAGGAGAGCGCACGAGGGAGCTTCC
AGGGGGAAACGCCTGGTATCTTTATAGTCCTGTCGGGTTT
CGCCACCTCTGACTTGAGCGTCGATTTTTGTGATGCTCGT
CAGGGGGGCGGAGCCTATGGAAAAACGCCAGCAACGCGGC
CTTTTTACGGTTCCTGGCCTTTTGCTGGCCTTTTGCTCAC
ATGTTCTTTCCTGCGTTATCCCCTGATTCTGTGGATAACC
GTATTACCGCCTTTGAGTGAGCTGATACCGCTCGCCGCAG
CCGAACGACCGAGCGCAGCGAGTCAGTGAGCGAGGAAGCG
GAAGAGCGCCCAATACGCAAACCGCCTCTCCCCGCGCGTT
GGCCGATTCATTAATGCAGCTGGCACGACAGGTTTCCCGA
CTGGAAAGCGGGCAGTGAGCGCAACGCAATTAATGTGAGT
TAGCTCACTCATTAGGCACCCCAGGCTTTACACTTTATGC
TTCCGGCTCGTATGTTGTGTGGAATTGTGAGCGGATAACA
ATTTCACACAGGAAACAGCTATGACCATGATTACGCCAGA
TTTAATTAAGGCCTTAATTAGGCTGCGCGCTCGCTCGCTC
ACTGAGGCCGCCCGGGCAAAGCCCGGGCGTCGGGCGACCT
TTGGTCGCCCGGCCTCAGTGAGCGAGCGAGCGCGCAGAGA
GGGAGTGGCCAACTCCATCACTAGGGGTTCCTTGTAGTTA
ATGATTAACCCGCCATGCTACTTATCTACGTAGCCATGCT
CTAGGAAGATCGGAATTCCTAGATCTACCTTGCTTCCTAG
CTGGGCCTTTCCTTCTCCTCTATAAATACCAGCTCTGGTA
TTTCGCCTTGGCAGCTGTTGCTGCTAGGGAGACGGCTGGC
TTGACATGCATCTCCTGACAAAACACAAACCCGTGGTGTG
AGTGGGTGTGGGCGGTGTGAGTAGGGGGATGAATCAGAGA
GGGGGCGAGGGAGACAGGGGCGCAGGAGTCAGGCAAAGGC
GATGCGGGGGTGCGACTACACGCAGTTGGAAACAGTCGTC
AGAAGATTCTGGAAACTATCTTGCTGGCTATAAACTTGAG
GGAAGCAGAAGGCCAACATTCCTCCCAAGGGAAACTGAGG
CTCAGAGTTAAAACCCAGGTATCAGTGATATGCATGTGCC
CCGGCCAGGGTCACTCTCTGACTAACCGGTACCTACCCTA
CAGGCCTACCTAGAGACTCTTTTGAAAGGATGGTAGAGAC
CTGTCCGGGCTTTGCCCACAGTCGTTGGAAACCTCAGCAT
TTTCTAGGCAACTTGTGCGAATAAAACACTTCGGGGGTCC
TTCTTGTTCATTCCAATAACCTAAAACCTCTCCTCGGAGA
AAATAGGGGGCCTCAAACAAACGAAATTCTCTAGCCCGCT
TTCCCCAGGATAAGGCAGGCATCCAAATGGAAAAAAAGGG
GCCGGCCGGGGGTCTCCTGTCAGCTCCTTGCCCTGTGAAA
CCCAGCAGGCCTGCCTGTCTTCTGTCCTCTTGGGGCTGTC
CAGGGGCGCAGGCCTCTTGCGGGGGAGCTGGCCTCCCCGC
CCCCTCGCCTGTGGCCGCCCTTTTCCTGGCAGGACAGAGG
GATCCTGCAGCTGTCAGGGGAGGGGCGCCGGGGGGTGATG
TCAGGAGGGCTACAAATAGTGCAGACAGCTAAGGGGCTCC
GTCACCCATCTTCACATCCACTCCAGCCGGCTGCCCGCCC
GCTGCCTCCTCTGTGCGTCCGCCCAGCCAGCCTCGTCCAC
GCCAAGCTTGCGGCCGCATTAAACGCCACCATGTCCCGCT
TGGATAAGAGCAAGGTAATAAATAGCGCACTCGAACTCCT
CAACGGCGTGGGCATCGAAGGTCTGACTACTCGAAAGCTC
GCCCAGAAATTGGGTGTGGAGCAACCTACATTGTATTGGC
ATGTCAAGAACAAAAGAGCCCTGCTGGACGCTCTTCCTAT
TGAAATGCTTGACAGGCATCACACTCATTCCTGCCCCCTT
GAGGTCGAGAGTTGGCAAGATTTTCTCCGAAACAATGCAA
AGTCCTACCGCTGCGCACTTTTGTCCCATAGGGATGGAGC
AAAAGTGCACCTGGGAACCAGGCCAACAGAGAAACAATAC
GAGACTCTCGAGAACCAGTTGGCTTTCTTGTGCCAACAGG
GGTTCTCACTTGAAAATGCCCTTTACGCACTGTCAGCCGT
TGGACATTTTACCCTGGGGTGCGTTCTTGAGGAGCAAGAA
CATCAGGTTGCTAAGGAGGAGCGCGAGACTCCAACCACTG
ATTCTATGCCACCTTTGCTGAAACAGGCCATTGAACTTTT
CGATAGACAGGGTGCTGAACCTGCCTTTCTCTTCGGGTTG
GAGCTGATTATTTGTGGTCTCGAAAAACAGCTGAAATGTG
AAAGTGGTGGCCCTACTGACGCCCTCGATGATTTCGACCT
GGATATGCTGCCAGCCGATGCACTTGATGATTTCGATTTG
GATATGCTTCCAGCCGACGCACTGGACGACTTCGATTTGG
ACATGCTTCCCGGTTAAACTAGTCTAGCAATCAACCTCTG
GATTACAAAATTTGTGAAAGATTGACTGGTATTCTTAACT
ATGTTGCTCCTTTTACGCTATGTGGATACGCTGCTTTAAT
GCCTTTGTATCATGCTATTGCTTCCCGTATGGCTTTCATT
TTCTCCTCCTTGTATAAATCCTGGTTAGTTCTTGCCACGG
CGGAACTCATCGCCGCCTGCCTTGCCCGCTGCTGGACAGG
GGCTCGGCTGTTGGGCACTGACAATTCCGTGGTGTTTATT
TGTGAAATTTGTGATGCTATTGCTTTATTTGTAACCATTC
TAGCTTTATTTGTGAAATTTGTGATGCTATTGCTTTATTT
GTAACCATTATAAGCTGCAATAAACAAGTTAACAACAACA
ATTGCATTCATTTTATGTTTCAGGTTCAGGGGGAGATGTG
GGAGGTTTTTTAAAGCGGGGGATCCAAATTCCCGATAAGG
ATCTTCCTAGAGCATGGCTACGTAGATAAGTAGCATGGCG
GGTTAATCATTAACTACAAGGAACCCCTAGTGATGGAGTT
GGCCACTCCCTCTCTGCGCGCTCGCTCGCTCACTGAGGCC
GGGCGACCAAAGGTCGCCCGACGCCCGGGCTTTGCCCGGG
CGGCCTCAGTGAGCGAGCGAGCGCGCAGCCTTAATTAACC
TAATTCACTGGCCGTCGTTTTACAACGTCGTGACTGGGAA
AACCCTGGCGTTACCCAACTTAATCGCCTTGCAGCACATC
CCCCTTTCGCCAGCTGGCGTAATAGCGAAGAGGCCCGCAC
CGATCGCCCTTCCCAACAGTTGCGCAGCCTGAATGGCGAA
TGGGACGCGCCCTGTAGCGGCGCATTAAGCGCGGCGGGTG
TGGTGGTTACGCGCAGCGTGACCGCTACACTTGCCAGCGC
CCTAGCGCCCGCTCCTTTCGCTTTCTTCCCTTCCTTTCTC
GCCACGTTCGCCGGCTTTCCCCGTCAAGCTCTAAATCGGG
GGCTCCCTTTAGGGTTCCGATTTAGTGCTTTACGGCACCT
CGACCCCAAAAAACTTGATTAGGGTGATGGTTCACGTAGT
GGGCCATCGCCCTGATAGACGGTTTTTCGCCCTTTGACGT
TGGAGTCCACGTTCTTTAATAGTGGACTCTTGTTCCAAAC
TGGAACAACACTCAACCCTATCTCGGTCTATTCTTTTGAT
TTATAAGGGATTTTGCCGATTTCGGCCTATTGGTTAAAAA
ATGAGCTGATTTAACAAAAATTTAACGCGAATTTTAACAA
AATATTAACGTTTATAATTTCAGGTGGCATCTTTCGGGGA
AATGTGCGCGGAACCCCTATTTGTTTATTTTTCTAAATAC
ATTCAAATATGTATCCGCTCATGAGACAATAACCCTGATA
AATGCTTCAATAATATTGAAAAAGGAAGAGTATGAGTATT
CAACATTTCCGTGTCGCCCTTATTCCCTTTTTTGCGGCAT
TTTGCCTTCCTGTTTTTGCTCACCCAGAAACGCTGGTGAA
AGTAAAAGATGCTGAAGATCAGTTGGGTGCACGAGTGGGT
TACATCGAACTGGATCTCAATAGTGGTAAGATCCTTGAGA
GTTTTCGCCCCGAAGAACGTTTTCCAATGATGAGCACTTT
TAAAGTTCTGCTATGTGGCGCGGTATTATCCCGTATTGAC
GCCGGGCAAGAGCAACTCGGTCGCCGCATACACTATTCTC
AGAATGACTTGGTTGAGTACTCACCAGTCACAGAAAAGCA
TCTTACGGATGGCATGACAGTAAGAGAA
pAAV2 CMV rtTA(V16) (SEQ ID NO: 31):
AAATTGTAAACGTTAATATTTTGTTAAAATTCGCGTTAAA
TTTTTGTTAAATCAGCTCATTTTTTAACCAATAGGCCGAA
ATCGGCAAAATCCCTTATAAATCAAAAGAATAGCCCGAGA
TAGGGTTGAGTGTTGTTCCAGTTTGGAACAAGAGTCCACT
ATTAAAGAACGTGGACTCCAACGTCAAAGGGCGAAAAACC
GTCTATCAGGGCGATGGCCCACTACGTGAACCATCACCCA
AATCAAGTTTTTTGGGGTCGAGGTGCCGTAAAGCACTAAA
TCGGAACCCTAAAGGGAGCCCCCGATTTAGAGCTTGACGG
GGAAAGCCGGCGAACGTGGCGAGAAAGGAAGGGAAGAAAG
CGAAAGGAGCGGGCGCTAGGGCGCTGGCAAGTGTAGCGG
TCACGCTGCGCGTAACCACCACACCCGCCGCGCTTAATGC
GCCGCTACAGGGCGCGTACTATGGTTGCTTTGACGTATGC
GGTGTGAAATACCGCACAGATGCGTAAGGAGAAAATACCG
CATCAGGCGCCCCTGCAGGCAGCTGCGCGCTCGCTCGCTC
ACTGAGGCCGCCCGGGCAAAGCCCGGGCGTCGGGCGACCT
TTGGTCGCCCGGCCTCAGTGAGCGAGCGAGCGCGCAGAGA
GGGAGTGGCCAACTCCATCACTAGGGGTTCCTGCGGCCGC
TCGGTCCGCACGATCTCAATTCGGCCATTACGGCCGGATC
CGGCTCGAGgagcttggcccattgcatacgttgtatccat
atcataatatgtacatttatattggctcatgtccaacatt
accgccatgttgacattgattattgactagttattaatag
taatcaattacggggtcattagttcatagcccatatatgg
agttccgcgttacataacttacggtaaatggcccgcctgg
ctgaccgcccaacgacccccgcccattgacgtcaataatg
acgtatgttcccatagtaacgccaatagggactttccatt
gacgtcaatgggtggagtatttacgctaaactgcccactt
ggcagtacatcaagtgtatcatatgccaagtacgccccct
attgacgtcaatgacggtaaatggcccgcctggcattatg
cccagtacatgaccttatgggactttcctacttggcagta
catctacgtattagtcatcgctattaccatggtgatgcgg
ttttggcagtacatcaatgggcgtggatagcggtttgact
cacggggatttccaagtctccaccccattgacgtcaatgg
gagtttgttttggcaccaaaatcaacgggactttccaaaa
tgtcgtaacaactccgccccattgacgcaaatgggcggta
ggcgtgtacggtgggaggtctatataagcagagctcgttt
agtgaaccgtcagatcgcctggagacgccatccacgctgt
tttgacctccatagaagacaccgggaccgatccagcctcc
gcggccccgaattcaccATGTCTAGACTGGACAAGAGCAA
AATCATAAACAGCGCTCTGGAATTACTCAATGGAGTCGGT
ATCGAAGGCCTGACGACAAGGAAACTCGCTCAAAAGCTGG
GAGTTGAGCAGCCTACCCTGTACTGGCACGTGAAGAACAA
GCGGGCCCTGCTCGATGCCCTGCCAATCGAGATGCTGGAC
AGGCATCATACCCACAGCTGCCCCCTGGAAGGCGAGTCAT
GGCAAGACTTTCTGCGGAACAACGCCAAGTCATACCGCTG
TGCTCTCCTCTCACATCGCGACGGGGCTAAAGTGCATCTC
GGCACCCGCCCAACAGAGAAACAGTACGAAACCCTGGAAA
ATCAGCTCGCGTTCCTGTGTCAGCAAGGCTTCTCCCTGGA
GAACGCACTGTACGCTCTGTCCGCCGTGGGCCACTTTACA
CTGGGCTGCGTATTGGAGGAACAGGAGCATCAAGTAGCAA
AAGAGGAAAGAGAGACACCTACCACCGATTCTATGCCCCC
ACTTCTGAAGCAAGCAATTGAGCTGTTCGACCGGCAGGGA
GCCGAACCTGCCTTCCTTTTCGGCCTGGAACTAATCATAT
GTGGCCTGGAGAAACAGCTAAAGTGCGAAAGCGGCGGGCC
GACCGACGCCCTTGACGATTTTGACTTAGACATGCTCCCA
GCCGATGCCCTTGACGACTTTGACCTTGATATGCTGCCTG
CTGACGCTCTTGACGATTTTGACCTTGACATGCTCCCCGG
GTAActaagtaaggatcATCTTAATTAAATCGATAAGGAT
CTGGCCGCCTCGGCCtaatcaacctctggattacaaaatt
tgtgaaagattgactggtattcttaactatgttgctcctt
ttacgctatgtggatacgctgctttaatgcctttgtatca
tgctattgcttcccgtatggctttcattttctcctccttg
tataaatcctggttgctgtctctttatgaggagttgtggc
ccgttgtcaggcaacgtggcgtggtgtgcactgtgtttgc
tgacgcaacccccactggttggggcattgccaccacctgt
cagctcctttccgggactttcgctttccccctccctattg
ccacggcggaactcatcgccgcctgccttgcccgctgctg
gacaggggctcggctgttgggcactgacaattccgtggtg
ttgtcggggaaatcatcgtcctttccttggctgctcgcct
gtgttgccacctggattctgcgcgggacgtccttctgcta
cgtcccttcggccctcaatccagcggaccttccttcccgc
ggcctgctgccggctctgcggcctcttccgcgtcttcgcc
ttcgccctcagacgagtcggatctccctttgggccgcctc
cccgcCAGACATGATAAGATACATTGATGAGTTTGGACAA
ACCACAACTAGAATGCAGTGAAAAAAATGCTTTATTTGTG
AAATTTGTGATGCTATTGCTTTATTTGTAACCATTATAAG
CTGCAATAAACAAGTTAACAACAACAATTGCATTCATTTT
ATGTTTCAGGTTCAGGGGGAGATGTGGGAGGTTTTTTAAA
GCAAGTAAAACCTCTACAAATGTGGTAACTAGCGCGTGCG
GCCGCAGGAACCCCTAGTGATGGAGTTGGCCACTCCCTCT
CTGCGCGCTCGCTCGCTCACTGAGGCCGGGCGACCAAAGG
TCGCCCGACGCCCGGGCTTTGCCCGGGCGGCCTCAGTGAG
CGAGCGAGCGCGCAGCTGCCTGCAGGACATGTGAGCAAAA
GGCCAGCAAAAGGCCAGGAACCGTAAAAAGGCCGCGTTGC
TGGCGTTTTTCCATAGGCTCCGCCCCCCTGACGAGCATCA
CAAAAATCGACGCTCAAGTCAGAGGTGGCGAAACCCGACA
GGACTATAAAGATACCAGGCGTTTCCCCCTGGAAGCTCCC
TCGTGCGCTCTCCTGTTCCGACCCTGCCGCTTACCGGATA
CCTGTCCGCCTTTCTCCCTTCGGGAAGCGTGGCGCTTTCT
CATAGCTCACGCTGTAGGTATCTCAGTTCGGTGTAGGTCG
TTCGCTCCAAGCTGGGCTGTGTGCACGAACCCCCCGTTCA
GCCCGACCGCTGCGCCTTATCCGGTAACTATCGTCTTGAG
TCCAACCCGGTAAGACACGACTTATCGCCACTGGCAGCAG
CCACTGGTAACAGGATTAGCAGAGCGAGGTATGTAGGCGG
TGCTACAGAGTTCTTGAAGTGGTGGCCTAACTACGGCTAC
ACTAGAAGGACAGTATTTGGTATCTGCGCTCTGCTGAAGC
CAGTTACCTTCGGAAAAAGAGTTGGTAGCTCTTGATCCGG
CAAACAAACCACCGCTGGTAGCGGTGGTTTTTTTGTTTGC
AAGCAGCAGATTACGCGCAGAAAAAAAGGATCTCAAGAAG
ATCCTTTGATCTTTTCTACGGGGTCTGACGCTCAGTGGAA
CGAAAACTCACGTTAAGGGATTTTGGTCATGAGATTATCA
AAAAGGATCTTCACCTAGATCCTTTTAAATTAAAAATGAA
GTTTTAAATCAATCTAAAGTATATATGAGTAAACTTGGTC
TGACAGTTACCAATGCTTAATCAGTGAGGCACCTATCTCA
GCGATCTGTCTATTTCGTTCATCCATAGTTGCCTGACTCC
CCGTCGTGTAGATAACTACGATACGGGAGGGCTTACCATC
TGGCCCCAGTGCTGCAATGATACCGCGAGACCCACGCTCA
CCGGCTCCAGATTTATCAGCAATAAACCAGCCAGCCGGAA
GGGCCGAGCGCAGAAGTGGTCCTGCAACTTTATCCGCCTC
CATCCAGTCTATTAATTGTTGCCGGGAAGCTAGAGTAAGT
AGTTCGCCAGTTAATAGTTTGCGCAACGTTGTTGCCATTG
CTACAGGCATCGTGGTGTCACGCTCGTCGTTTGGTATGGC
TTCATTCAGCTCCGGTTCCCAACGATCAAGGCGAGTTACA
TGATCCCCCATGTTGTGCAAAAAAGCGGTTAGCTCCTTCG
GTCCTCCGATCGTTGTCAGAAGTAAGTTGGCCGCAGTGTT
ATCACTCATGGTTATGGCAGCACTGCATAATTCTCTTACT
GTCATGCCATCCGTAAGATGCTTTTCTGTGACTGGTGAGT
ACTCAACCAAGTCATTCTGAGAATAGTGTATGCGGCGACC
GAGTTGCTCTTGCCCGGCGTCAATACGGGATAATACCGCG
CCACATAGCAGAACTTTAAAAGTGCTCATCATTGGAAAAC
GTTCTTCGGGGCGAAAACTCTCAAGGATCTTACCGCTGTT
GAGATCCAGTTCGATGTAACCCACTCGTGCACCCAACTGA
TCTTCAGCATCTTTTACTTTCACCAGCGTTTCTGGGTGAG
CAAAAACAGGAAGGCAAAATGCCGCAAAAAAGGGAATAAG
GGCGACACGGAAATGTTGAATACTCATACTCTTCCTTTTT
CAATATTATTGAAGCATTTATCAGGGTTATTGTCTCATGA
GCGGATACATATTTGAATGTATTTAGAAAAATAAACAAAT
AGGGGTTCCGCGCACATTTCCCCGAAAAGTGCCACCTGAC
GTCTAAGAAACCATTATTATCATGACATTAACCTATAAAA
ATAGGCGTATCACGAGGCCCTTTCGTCTCGCGCGTTTCGG
TGATGACGGTGAAAACCTCTGACACATGCAGCTCCCGGAG
ACGGTCACAGCTTGTCTGTAAGCGGATGCCGGGAGCAGAC
AAGCCCGTCAGGGCGCGTCAGCGGGTGTTGGCGGGTGTCG
GGGCTGGCTTAACTATGCGGCATCAGAGCAGATTGTACTG
AGAGTGCACCATA
CAG-tTA (SEQ ID NO: 32):
CCTGCAGGCAGCTGCGCGCTCGCTCGCTCACTGAGGCCGC
CCGGGCAAAGCCCGGGCGTCGGGCGACCTTTGGTCGCCCG
GCCTCAGTGAGCGAGCGAGCGCGCAGAGAGGGAGTGGCCA
ACTCCATCACTAGGGGTTCCTGCGGCCGCACGCGTGGAGC
TAGTTATTAATAGTAATCAATTACGGGGTCATTAGTTCAT
AGCCCATATATGGAGTTCCGCGTTACATAACTTACGGTAA
ATGGCCCGCCTGGCTGACCGCCCAACGACCCCCGCCCATT
GACGTCAATAATGACGTATGTTCCCATAGTAACGTCAATA
GGGACTTTCCATTGACGTCAATGGGTGGAGTATTTACGGT
AAACTGCCCACTTGGCAGTACATCAAGTGTATCATATGCC
AAGTACGCCCCCTATTGACGTCAATGACGGTAAATGGCCC
GCCTGGCATTATGCCCAGTACATGACCTTATGGGACTTTC
CTACTTGGCAGTACATCTACGTATTAGTCATCGCTATTAC
CATGGTGATGCGGTTTTGGCAGTACATCAATGGGCGTGGA
TAGCGGTTTGACTCACGGGGATTTCCAAGTCTCCACCCCA
TTGACGTCAATGGGAGTTTGTTTTGCACCAAAATCAACGG
GACTTTCCAAAATGTCGTAACAACTCCGCCCCATTGACGC
AAATGGGCGGTAGGCGTGTACGGTGGGAGGTCTATATAAG
CAGAGCTCGTTTAGTGAACCGTCAGATCGCCTGGAGACGC
CATCCACGCTGTTTTGACCTCCATAGAAGACACCGGGACC
GATCCAGCCTCCGCGGATTCGAATCCCGGCCGGGAACGGT
GCATTGGAACGCGGATTCCCCGTGCCAAGAGTGACGTAAG
TACCGCCTATAGAGTCTATAGGCCCACAAAAAATGCTTTC
TTCTTTTAATATACTTTTTTGTTTATCTTATTTCTAATAC
TTTCCCTAATCTCTTTCTTTCAGGGCAATAATGATACAAT
GTATCATGCCTCTTTGCACCATTCTAAAGAATAACAGTGA
TAATTTCTGGGTTAAGGCAATAGCAATATTTCTGCATATA
AATATTTCTGCATATAAATTGTAACTGATGTAAGAGGTTT
CATATTGCTAATAGCAGCTACAATCCAGCTACCATTCTGC
TTTTATTTTATGGTTGGGATAAGGCTGGATTATTCTGAGT
CCAAGCTAGGCCCTTTTGCTAATCATGTTCATACCTCTTA
TCTTCCTCCCACAGCTCCTGGGCAACGTGCTGGTCTGTGT
GCTGGCCCATCACTTTGGCAAAGAATTGGGATTCGAACAT
CGATTGAATTCATGTCTAGACTGGACAAGAGCAAAGTCAT
AAACTCTGCTCTGGAATTACTCAATGAAGTCGGTATCGAA
GGCCTGACGACAAGGAAACTCGCTCAAAAGCTGGGAGTTG
AGCAGCCTACCCTGTACTGGCACGTGAAGAACAAGCGGGC
CCTGCTCGATGCCCTGGCAATCGAGATGCTGGACAGGCAT
CATACCCACTTCTGCCCCCTGGAAGGCGAGTCATGGCAAG
ACTTTCTGCGGAACAACGCCAAGTCATTCCGCTGTGCTCT
CCTCTCACATCGCGACGGGGCTAAAGTGCATCTCGGCACC
CGCCCAACAGAGAAACAGTACGAAACCCTGGAAAATCAGC
TCGCGTTCCTGTGTCAGCAAGGCTTCTCCCTGGAGAACGC
ACTGTACGCTCTGTCCGCCGTGGGCCACTTTACACTGGGC
TGCGTATTGGAGGATCAGGAGCATCAAGTAGCAAAAGAGG
AAAGAGAGACACCTACCACCGATTCTATGCCCCCACTTCT
GAGACAAGCAATTGAGCTGTTCGACCATCAGGGAGCCGAA
CCTGCCTTCCTTTTCGGCCTGGAACTAATCATATGTGGCC
TGGAGAAACAGCTAAAGTGCGAAAGCGGCGGGCCGGCCGA
CGCCCTTGACGATTTTGACTTAGACATGCTCCCAGCCGAT
GCCCTTGACGACTTTGACCTTGATATGCTGCCTGCTGACG
CTCTTGACGATTTTGACCTTGACATGCTCCCCGGATGAGG
ATCCTCTAGAGTCGACCTGCAGAAGCTTGCCTCGAGCAGC
GCTGCTCGAGAGATCTACGGGTGGCATCCCTGTGACCCCT
CCCCAGTGCCTCTCCTGGCCCTGGAAGTTGCCACTCCAGT
GCCCACCAGCCTTGTCCTAATAAAATTAAGTTGCATCATT
TTGTCTGACTAGGTGTCCTTCTATAATATTATGGGGTGGA
GGGGGGTGGTATGGAGCAAGGGGCAAGTTGGGAAGACAAC
CTGTAGGGCCTGCGGGGTCTATTGGGAACCAAGCTGGAGT
GCAGTGGCACAATCTTGGCTCACTGCAATCTCCGCCTCCT
GGGTTCAAGCGATTCTCCTGCCTCAGCCTCCCGAGTTGTT
GGGATTCCAGGCATGCATGACCAGGCTCAGCTAATTTTTG
TTTTTTTGGTAGAGACGGGGTTTCACCATATTGGCCAGGC
TGGTCTCCAACTCCTAATCTCAGGTGATCTACCCACCTTG
GCCTCCCAAATTGCTGGGATTACAGGCGTGAACCACTGCT
CCCTTCCCTGTCCTTCTGATTTTGTAGGTAACCACGTGCG
GACCGAGCGGCCGCAGGAACCCCTAGTGATGGAGTTGGCC
ACTCCCTCTCTGCGCGCTCGCTCGCTCACTGAGGCCGGGC
GACCAAAGGTCGCCCGACGCCCGGGCTTTGCCCGGGCGGC
CTCAGTGAGCGAGCGAGCGCGCAGCTGCCTGCAGGGGCGC
CTGATGCGGTATTTTCTCCTTACGCATCTGTGCGGTATTT
CACACCGCATACGTCAAAGCAACCATAGTACGCGCCCTGT
AGCGGCGCATTAAGCGCGGCGGGTGTGGTGGTTACGCGCA
GCGTGACCGCTACACTTGCCAGCGCCCTAGCGCCCGCTCC
TTTCGCTTTCTTCCCTTCCTTTCTCGCCACGTTCGCCGGC
TTTCCCCGTCAAGCTCTAAATCGGGGGCTCCCTTTAGGGT
TCCGATTTAGTGCTTTACGGCACCTCGACCCCAAAAAACT
TGATTTGGGTGATGGTTCACGTAGTGGGCCATCGCCCTGA
TAGACGGTTTTTCGCCCTTTGACGTTGGAGTCCACGTTCT
TTAATAGTGGACTCTTGTTCCAAACTGGAACAACACTCAA
CCCTATCTCGGGCTATTCTTTTGATTTATAAGGGATTTTG
CCGATTTCGGCCTATTGGTTAAAAAATGAGCTGATTTAAC
AAAAATTTAACGCGAATTTTAACAAAATATTAACGTTTAC
AATTTTATGGTGCACTCTCAGTACAATCTGCTCTGATGCC
GCATAGTTAAGCCAGCCCCGACACCCGCCAACACCCGCTG
ACGCGCCCTGACGGGCTTGTCTGCTCCCGGCATCCGCTTA
CAGACAAGCTGTGACCGTCTCCGGGAGCTGCATGTGTCAG
AGGTTTTCACCGTCATCACCGAAACGCGCGAGACGAAAGG
GCCTCGTGATACGCCTATTTTTATAGGTTAATGTCATGAT
AATAATGGTTTCTTAGACGTCAGGTGGCACTTTTCGGGGA
AATGTGCGCGGAACCCCTATTTGTTTATTTTTCTAAATAC
ATTCAAATATGTATCCGCTCATGAGACAATAACCCTGATA
AATGCTTCAATAATATTGAAAAAGGAAGAGTATGAGTATT
CAACATTTCCGTGTCGCCCTTATTCCCTTTTTTGCGGCAT
TTTGCCTTCCTGTTTTTGCTCACCCAGAAACGCTGGTGAA
AGTAAAAGATGCTGAAGATCAGTTGGGTGCACGAGTGGGT
TACATCGAACTGGATCTCAACAGCGGTAAGATCCTTGAGA
GTTTTCGCCCCGAAGAACGTTTTCCAATGATGAGCACTTT
TAAAGTTCTGCTATGTGGCGCGGTATTATCCCGTATTGAC
GCCGGGCAAGAGCAACTCGGTCGCCGCATACACTATTCTC
AGAATGACTTGGTTGAGTACTCACCAGTCACAGAAAAGCA
TCTTACGGATGGCATGACAGTAAGAGAATTATGCAGTGCT
GCCATAACCATGAGTGATAACACTGCGGCCAACTTACTTC
TGACAACGATCGGAGGACCGAAGGAGCTAACCGCTTTTTT
GCACAACATGGGGGATCATGTAACTCGCCTTGATCGTTGG
GAACCGGAGCTGAATGAAGCCATACCAAACGACGAGCGTG
ACACCACGATGCCTGTAGCAATGGCAACAACGTTGCGCAA
ACTATTAACTGGCGAACTACTTACTCTAGCTTCCCGGCAA
CAATTAATAGACTGGATGGAGGCGGATAAAGTTGCAGGAC
CACTTCTGCGCTCGGCCCTTCCGGCTGGCTGGTTTATTGC
TGATAAATCTGGAGCCGGTGAGCGTGGGTCTCGCGGTATC
ATTGCAGCACTGGGGCCAGATGGTAAGCCCTCCCGTATCG
TAGTTATCTACACGACGGGGAGTCAGGCAACTATGGATGA
ACGAAATAGACAGATCGCTGAGATAGGTGCCTCACTGATT
AAGCATTGGTAACTGTCAGACCAAGTTTACTCATATATAC
TTTAGATTGATTTAAAACTTCATTTTTAATTTAAAAGGAT
CTAGGTGAAGATCCTTTTTGATAATCTCATGACCAAAATC
CCTTAACGTGAGTTTTCGTTCCACTGAGCGTCAGACCCCG
TAGAAAAGATCAAAGGATCTTCTTGAGATCCTTTTTTTCT
GCGCGTAATCTGCTGCTTGCAAACAAAAAAACCACCGCTA
CCAGCGGTGGTTTGTTTGCCGGATCAAGAGCTACCAACTC
TTTTTCCGAAGGTAACTGGCTTCAGCAGAGCGCAGATACC
AAATACTGTCCTTCTAGTGTAGCCGTAGTTAGGCCACCAC
TTCAAGAACTCTGTAGCACCGCCTACATACCTCGCTCTGC
TAATCCTGTTACCAGTGGCTGCTGCCAGTGGCGATAAGTC
GTGTCTTACCGGGTTGGACTCAAGACGATAGTTACCGGAT
AAGGCGCAGCGGTCGGGCTGAACGGGGGGTTCGTGCACAC
AGCCCAGCTTGGAGCGAACGACCTACACCGAACTGAGATA
CCTACAGCGTGAGCTATGAGAAAGCGCCACGCTTCCCGAA
GGGAGAAAGGCGGACAGGTATCCGGTAAGCGGCAGGGTCG
GAACAGGAGAGCGCACGAGGGAGCTTCCAGGGGGAAACGC
CTGGTATCTTTATAGTCCTGTCGGGTTTCGCCACCTCTGA
CTTGAGCGTCGATTTTTGTGATGCTCGTCAGGGGGGCGGA
GCCTATGGAAAAACGCCAGCAACGCGGCCTTTTTACGGTT
CCTGGCCTTTTGCTGGCCTTTTGCTCACATGT
pAAV-Tet-O-OSK-SV40LpA
(or pAAV-TRE2-OSK-SV40LpA)
(SEQ ID NO: 33):
ttatgcagtgctgccataaccatgagtgataacactgcgg
ccaacttacttctgacaacgatcggaggaccgaaggagct
aaccgcttttttgcacaacatgggggatcatgtaactcgc
cttgatcgttgggaaccggagctgaatgaagccataccaa
acgacgagcgtgacaccacgatgcctgtagtaatggtaac
aacgttgcgcaaactattaactggcgaactacttactcta
gcttcccggcaacaattaatagactggatggaggcggata
aagttgcaggaccacttctgcgctcggcccttccggctgg
ctggtttattgctgataaatctggagccggtgagcgtggg
tctcgcggtatcattgcagcactggggccagatggtaagc
cctcccgtatcgtagttatctacacgacggggagtcaggc
aactatggatgaacgaaatagacagatcgctgagataggt
gcctcactgattaagcattggtaactgtcagaccaagttt
actcatatatactttagattgatttaaaacttcattttta
atttaaaaggatctaggtgaagatcattttgataatctca
tgaccaaaatcccttaacgtgagttttcgttccactgagc
gtcagaccccgtagaaaagatcaaaggatcttcttgagat
cctttttttctgcgcgtaatctgctgcttgcaaacaaaaa
aaccaccgctaccagcggtggtttgtttgccggatcaaga
gctaccaactctttttccgaaggtaactggcttcagcaga
gcgcagataccaaatactgtccttctagtgtagccgtagt
taggccaccacttcaagaactctgtagcaccgcctacata
cctcgctctgctaatcctgttaccagtggctgctgccagt
ggcgataagtcgtgtcttaccgggttggactcaagacgat
agttaccggataaggcgcagcggtcgggctgaacgggggg
ttcgtgcacacagcccagcttggagcgaacgacctacacc
gaactgagatacctacagcgtgagctatgagaaagcgcca
cgcttcccgaagggagaaaggcggacaggtatccggtaag
cggcagggtcggaacaggagagcgcacgagggagcttcca
gggggaaacgcctggtatctttatagtcctgtcgggtttc
gccacctctgacttgagcgtcgatttttgtgatgctcgtc
aggggggcggagcctatggaaaaacgccagcaacgcggcc
tttttacggttcctggccttttgctggccttttgctcaca
tgttctttcctgcgttatcccctgattctgtggataaccg
tattaccgcctttgagtgagctgataccgctcgccgcagc
cgaacgaccgagcgcagcgagtcagtgagcgaggaagcgg
aagagcgcccaatacgcaaaccgcctctccccgcgcgttg
gccgattcattaatgcagctggcacgacaggtttcccgac
tggaaagcgggcagtgagcgcaacgcaattaatgtgagtt
agctcactcattaggcaccccaggctttacactttatgct
tccggctcgtatgttgtgtggaattgtgagcggataacaa
tttcacacaggaaacagctatgaccatgattacgccagat
ttaattaaggccttaattaggctgcgcgctcgctcgctca
ctgaggccgcccgggcaaagcccgggcgtcgggcgacctt
tggtcgcccggcctcagtgagcgagcgagcgcgcagagag
ggagtggccaactccatcactaggggttccttgtagttaa
tgattaacccgccatgctacttatctacgtagccatgctc
taggaagatcggaattcgtacacgcctacctcgacccatc
aagtgccacctgacgtctccctatcagtgatagagaagtc
gacacgtctcgagctccctatcagtgatagagaaggtacg
tctagaacgtctccctatcagtgatagagaagtcgacacg
tctcgagctccctatcagtgatagagaaggtacgtctaga
acgtctccctatcagtgatagagaagtcgacacgtctcga
gctccctatcagtgatagagaaggtacgtctagaacgtct
ccctatcagtgatagagaagtcgacacgtctcgagctccc
tatcagtgatagagaaggtaccccctatataagcagagct
cgtttagtgaaccgtcagatcgcctggagacgccatccac
gctgttttgacctccatagaagacaccgggaccgatccag
cctggatcgcggccgcgccaccatggctggacacctggct
tcagacttcgccttctcacccccaccaggtgggggtgatg
ggtcagcagggctggagccgggctgggtggatcctcgaac
ctggctaagcttccaagggcctccaggtgggcctggaatc
ggaccaggctcagaggtattggggatctccccatgtccgc
ccgcatacgagttctgcggagggatggcatactgtggacc
tcaggttggactgggcctagtcccccaagttggcgtggag
actttgcagcctgagggccaggcaggagcacgagtggaaa
gcaactcagagggaacctcctctgagccctgtgccgaccg
ccccaatgccgtgaagttggagaaggtggaaccaactccc
gaggagtcccaggacatgaaagccctgcagaaggagctag
aacagtttgccaagctgctgaagcagaagaggatcacctt
ggggtacacccaggccgacgtggggctcaccctgggcgtt
ctctttggaaaggtgttcagccagaccaccatctgtcgct
tcgaggccttgcagctcagccttaagaacatgtgtaagct
gcggcccctgctggagaagtgggtggaggaagccgacaac
aatgagaaccttcaggagatatgcaaatcggagaccctgg
tgcaggcccggaagagaaagcgaactagcattgagaaccg
tgtgaggtggagtctggagaccatgtttctgaagtgcccg
aagccctccctacagcagatcactcacatcgccaatcagc
ttgggctagagaaggatgtggttcgagtatggttctgtaa
ccggcgccagaagggcaaaagatcaagtattgagtattcc
caacgagaagagtatgaggctacagggacacctttcccag
ggggggctgtatcctttcctctgcccccaggtccccactt
tggcaccccaggctatggaagcccccacttcaccacactc
tactcagtcccttttcctgagggcgaggcctttccctctg
ttcccgtcactgctctgggctctcccatgcattcaaacgc
tagcggcagcggcgccacgaacttctctctgttaaagcaa
gcaggagatgttgaagaaaaccccgggcctgcatgcatgt
ataacatgatggagacggagctgaagccgccgggcccgca
gcaagcttcggggggcggcggcggaggaggcaacgccacg
gcggcggcgaccggcggcaaccagaagaacagcccggacc
gcgtcaagaggcccatgaacgccttcatggtatggtcccg
ggggcagcggcgtaagatggcccaggagaaccccaagatg
cacaactcggagatcagcaagcgcctgggcgcggagtgga
aacttttgtccgagaccgagaagcggccgttcatcgacga
ggccaagcggctgcgcgctctgcacatgaaggagcacccg
gattataaataccggccgcggcggaaaaccaagacgctca
tgaagaaggataagtacacgcttcccggaggcttgctggc
ccccggcgggaacagcatggcgagcggggttggggtgggc
gccggcctgggtgcgggcgtgaaccagcgcatggacagct
acgcgcacatgaacggctggagcaacggcagctacagcat
gatgcaggagcagctgggctacccgcagcacccgggcctc
aacgctcacggcgcggcacagatgcaaccgatgcaccgct
acgacgtcagcgccctgcagtacaactccatgaccagctc
gcagacctacatgaacggctcgcccacctacagcatgtcc
tactcgcagcagggcacccccggtatggcgctgggctcca
tgggctctgtggtcaagtccgaggccagctccagcccccc
cgtggttacctcttcctcccactccagggcgccctgccag
gccggggacctccgggacatgatcagcatgtacctccccg
gcgccgaggtgccggagcccgctgcgcccagtagactgca
catggcccagcactaccagagcggcccggtgcccggcacg
gccattaacggcacactgcccctgtcgcacatggcatgcg
gctccggcgagggcaggggaagtcttctaacatgcgggga
cgtggaggaaaatcccggcccactcgagatgaggcagcca
cctggcgagtctgacatggctgtcagcgacgctctgctcc
cgtccttctccacgttcgcgtccggcccggcgggaaggga
gaagacactgcgtccagcaggtgccccgactaaccgttgg
cgtgaggaactctctcacatgaagcgacttcccccacttc
ccggccgcccctacgacctggcggcgacggtggccacaga
cctggagagtggcggagctggtgcagcttgcagcagtaac
aacccggccctcctagcccggagggagaccgaggagttca
acgacctcctggacctagactttatcctttccaactcgct
aacccaccaggaatcggtggccgccaccgtgaccacctcg
gcgtcagcttcatcctcgtcttccccagcgagcagcggcc
ctgccagcgcgccctccacctgcagcttcagctatccgat
ccgggccgggggtgacccgggcgtggctgccagcaacaca
ggtggagggctcctctacagccgagaatctgcgccacctc
ccacggcccccttcaacctggcggacatcaatgacgtgag
cccctcgggcggcttcgtggctgagctcctgcggccggag
ttggacccagtatacattccgccacagcagcctcagccgc
caggtggcgggctgatgggcaagtttgtgctgaaggcgtc
tctgaccacccctggcagcgagtacagcagcccttcggtc
atcagtgttagcaaaggaagcccagacggcagccaccccg
tggtagtggcgccctacagcggtggcccgccgcgcatgtg
ccccaagattaagcaagaggcggtcccgtcctgcacggtc
agccggtccctagaggcccatttgagcgctggaccccagc
tcagcaacggccaccggcccaacacacacgacttccccct
ggggcggcagctccccaccaggactacccctacactgagt
cccgaggaactgctgaacagcagggactgtcaccctggcc
tgcctcttcccccaggattccatccccatccggggcccaa
ctaccctcctttcctgccagaccagatgcagtcacaagtc
ccctctctccattatcaagagctcatgccaccgggttcct
gcctgccagaggagcccaagccaaagaggggaagaaggtc
gtggccccggaaaagaacagccacccacacttgtgactat
gcaggctgtggcaaaacctataccaagagttctcatctca
aggcacacctgcgaactcacacaggcgagaaaccttacca
ctgtgactgggacggctgtgggtggaaattcgcccgctcc
gatgaactgaccaggcactaccgcaaacacacagggcacc
ggccctttcagtgccagaagtgcgacagggccttttccag
gtcggaccaccttgccttacacatgaagaggcactaaatg
actagtctagcaatcaacctctggattacaaaatttgtga
aagattgactggtattcttaactatgttgctccttttacg
ctatgtggatacgctgctttaatgcctttgtatcatgcta
ttgcttcccgtatggctttcattttctcctccttgtataa
atcctggttagttcttgccacggcggaactcatcgccgcc
tgccttgcccgctgctggacaggggctcggctgttgggca
ctgacaattccgtggtgtttatttgtgaaatttgtgatgc
tattgctttatttgtaaccattctagctttatttgtgaaa
tttgtgatgctattgctttatttgtaaccattataagctg
caataaacaagttaacaacaacaattgcattcattttatg
tttcaggttcagggggagatgtgggaggttttttaaagcg
ggggatccaaattcccgataaggatcttcctagagcatgg
ctacgtagataagtagcatggcgggttaatcattaactac
aaggaacccctagtgatggagttggccactccctctctgc
gcgctcgctcgctcactgaggccgggcgaccaaaggtcgc
ccgacgcccgggctttgcccgggcggcctcagtgagcgag
cgagcgcgcagccttaattaacctaattcactggccgtcg
ttttacaacgtcgtgactgggaaaaccctggcgttaccca
acttaatcgccttgcagcacatccccctttcgccagctgg
cgtaatagcgaagaggcccgcaccgatcgcccttcccaac
agttgcgcagcctgaatggcgaatgggacgcgccctgtag
cggcgcattaagcgcggcgggtgtggtggttacgcgcagc
gtgaccgctacacttgccagcgccctagcgcccgctcctt
tcgctttcttcccttcctttctcgccacgttcgccggctt
tccccgtcaagctctaaatcgggggctccctttagggttc
cgatttagtgctttacggcacctcgaccccaaaaaacttg
attagggtgatggttcacgtagtgggccatcgccctgata
gacggtttttcgccctttgacgttggagtccacgttcttt
aatagtggactcttgttccaaactggaacaacactcaacc
ctatctcggtctattcttttgatttataagggattttgcc
gatttcggcctattggttaaaaaatgagctgatttaacaa
aaatttaacgcgaattttaacaaaatattaacgtttataa
tttcaggtggcatctttcggggaaatgtgcgcggaacccc
tatttgtttatttttctaaatacattcaaatatgtatccg
ctcatgagacaataaccctgataaatgcttcaataatatt
gaaaaaggaagagtatgagtattcaacatttccgtgtcgc
ccttattcccttttttgcggcattttgccttcctgttttt
gctcacccagaaacgctggtgaaagtaaaagatgctgaag
atcagttgggtgcacgagtgggttacatcgaactggatct
caatagtggtaagatccttgagagttttcgccccgaagaa
cgttttccaatgatgagcacttttaaagttctgctatgtg
gcgcggtattatcccgtattgacgccgggcaagagcaact
cggtcgccgcatacactattctcagaatgacttggttgag
tactcaccagtcacagaaaagcatcttacggatggcatga
cagtaagagaa
VP64, 4 repeats of VP16 (SEQ ID NO: 34)
(Non-limiting example of a
transactivation domain):
GAGGCCAGCGGTTCCGGACGGGCTGACGCATTGGACGATT
TTGATCTGGATATGCTGGGAAGTGACGCCCTCGATGATTT
TGACCTTGACATGCTTGGTTCGGATGCCCTTGATGACTTT
GACCTCGACATGCTCGGCAGTGACGCCCTTGATGATTTCG
ACCTGGACATGCTGATTAACTCTAGA
P65 (SEQ ID NO: 35) (Non-limiting example
of a transactivation domain):
AGCCAGTACCTGCCCGACACCGACGACCGGCACCGGATCG
AGGAAAAGCGGAAGCGGACCTACGAGACATTCAAGAGCAT
CATGAAGAAGTCCCCCTTCAGCGGCCCCACCGACCCTAGA
CCTCCACCTAGAAGAATCGCCGTGCCCAGCAGATCCAGCG
CCAGCGTGCCAAAACCTGCCCCCCAGCCTTACCCCTTCAC
CAGCAGCCTGAGCACCATCAACTACGACGAGTTCCCTACC
ATGGTGTTCCCCAGCGGCCAGATCTCTCAGGCCTCTGCTC
TGGCTCCAGCCCCTCCTCAGGTGCTGCCTCAGGCTCCTGC
TCCTGCACCAGCTCCAGCCATGGTGTCTGCACTGGCTCAG
GCACCAGCACCCGTGCCTGTGCTGGCTCCTGGACCTCCA
CAGGCTGTGGCTCCACCAGCCCCTAAACCTACACAGGCCG
GCGAGGGCACACTGTCTGAAGCTCTGCTGCAGCTGCAGTT
CGACGACGAGGATCTGGGAGCCCTGCTGGGAAACAGCACC
GATCCTGCCGTGTTCACCGACCTGGCCAGCGTGGACAACA
GCGAGTTCCAGCAGCTGCTGAACCAGGGCATCCCTGTGGC
CCCTCACACCACCGAGCCCATGCTGATGGAATACCCCGAG
GCCATCACCCGGCTCGTGACAGGCGCTCAGAGGCCTCCTG
ATCCAGCTCCTGCCCCTCTGGGAGCACCAGGCCTGCCTAA
TGGACTGCTGTCTGGCGACGAGGACTTCAGCTCTATCGC
CGATATGGATTTCTCAGCCTTGCTG
RTA (SEQ ID NO: 36) (Non-limiting example
of a transactivation domain):
CGGGATTCCAGGGAAGGGATGTTTTTGCCGAAGCCTGAGG
CCGGCTCCGCTATTAGTGACGTGTTTGAGGGCCGCGAGGT
GTGCCAGCCAAAACGAATCCGGCCATTTCATCCTCCAGGA
AGTCCATGGGCCAACCGCCCACTCCCCGCCAGCCTCGCAC
CAACACCAACCGGTCCAGTACATGAGCCAGTCGGGTCACT
GACCCCGGCACCAGTCCCTCAGCCACTGGATCCAGCGCCC
GCAGTGACTCCCGAGGCCAGTCACCTGTTGGAGGATCCCG
ATGAAGAGACGAGCCAGGCTGTCAAAGCCCTTCGGGAGAT
GGCCGATACTGTGATTCCCCAGAAGGAAGAGGCTGCAATC
TGTGGCCAAATGGACCTTTCCCATCCGCCCCCAAGGGGC
CATCTGGATGAGCTGACAACCACACTTGAGTCCATGACCG
AGGATCTGAACCTGGACTCACCCCTGACCCCGGAATTGAA
CGAGATTCTGGATACCTTCCTGAACGACGAGTGCCTCTTG
CATGCCATGCATATCAGCACAGGACTGTCCATCTTCGACA
CATCTCTGTTT
MPH MS2-P65-HSF1 (SEQ ID NO: 37)
(Non-limiting example of a
transactivationdomain):
GCTTCAAACTTTACTCAGTTCGTGCTCGTGGACAATGGTG
GGACAGGGGATGTGACAGTGGCTCCTTCTAATTTCGCTAA
TGGGGTGGCAGAGTGGATCAGCTCCAACTCACGGAGCCAG
GCCTACAAGGTGACATGCAGCGTCAGGCAGTCTAGTGCCC
AGAAGAGAAAGTATACCATCAAGGTGGAGGTCCCCAAAGT
GGCTACCCAGACAGTGGGCGGAGTCGAACTGCCTGTCGCC
GCTTGGAGGTCCTACCTGAACATGGAGCTCACTATCCCAA
TTTTCGCTACCAATTCTGACTGTGAACTCATCGTGAAGGC
AATGCAGGGGCTCCTCAAAGACGGTAATCCTATCCCTTCC
GCCATCGCCGCTAACTCAGGTATCTACAGCGCTGGAGGA
GGTGGAAGCGGAGGAGGAGGAAGCGGAGGAGGAGGTAGCG
GACCTAAGAAAAAGAGGAAGGTGGCGGCCGCTGGATCCCC
TTCAGGGCAGATCAGCAACCAGGCCCTGGCTCTGGCCCCT
AGCTCCGCTCCAGTGCTGGCCCAGACTATGGTGCCCTCTA
GTGCTATGGTGCCTCTGGCCCAGCCACCTGCTCCAGCCCC
TGTGCTGACCCCAGGACCACCCCAGTCACTGAGCGCTCCA
GTGCCCAAGTCTACACAGGCCGGCGAGGGGACTCTGAGTG
AAGCTCTGCTGCACCTGCAGTTCGACGCTGATGAGGACCT
GGGAGCTCTGCTGGGGAACAGCACCGATCCCGGAGTGTT
CACAGATCTGGCCTCCGTGGACAACTCTGAGTTTCAGCAG
CTGCTGAATCAGGGCGTGTCCATGTCTCATAGTACAGCCG
AACCAATGCTGATGGAGTACCCCGAAGCCATTACCCGGCT
GGTGACCGGCAGCCAGCGGCCCCCCGACCCCGCTCCAACT
CCCCTGGGAACCAGCGGCCTGCCTAATGGGCTGTCCGGAG
ATGAAGACTTCTCAAGCATCGCTGATATGGACTTTAGTGC
CCTGCTGTCACAGATTTCCTCTAGTGGGCAGGGAGGAGGT
GGAAGCGGCTTCAGCGTGGACACCAGTGCCCTGCTGGACC
TGTTCAGCCCCTCGGTGACCGTGCCCGACATGAGCCTGC
CTGACCTTGACAGCAGCCTGGCCAGTATCCAAGAGCTCCT
GTCTCCCCAGGAGCCCCCCAGGCCTCCCGAGGCAGAGAAC
AGCAGCCCGGATTCAGGGAAGCAGCTGGTGCACTACACAG
CGCAGCCGCTGTTCCTGCTGGACCCCGGCTCCGTGGACAC
CGGGAGCAACGACCTGCCGGTGCTGTTTGAGCTGGGAGAG
GGCTCCTACTTCTCCGAAGGGGACGGCTTCGCCGAGGACC
CCACCATCTCCCTGCTGACAGGCTCGGAGCCTCCCAAAGC
CAAGGACCCCACTGTCTCC

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A mutant reverse tetracycline transactivator (rtTA), which comprises mutations at residues corresponding to the following positions in rtTA3 (SEQ ID NO: 11):
(a) G72;
(b) G12;
(c) F67; and
(d) R171.

2. The mutant rtTA of claim 1, wherein:
(a) the G72 mutation is a G72V mutation, a G72I mutation, a G72L mutation, or a G72P mutation;
(b) the G12 mutation is a G12S mutation or a G12T mutation;
(c) the F67 mutation is a F67S mutation or a F67T mutation; and
(d) the R171 mutation is a R171K mutation or a R171H mutation.

3. The mutant rtTA of claim 1, further comprising at least one other mutation corresponding to a position in rtTA3 (SEQ ID NO: 11): that is not one of (a)-(d).

4. The mutant rtTA of claim 3, wherein the at least one other mutation is a deletion, insertion, point mutation, or a truncation mutation.

5. The mutant rtTA of claim 1, wherein:
(a) the G72 mutation is a G72V mutation or a G72P mutation;
(b) the G12 mutation is a G12S mutation;
(c) the F67 mutation is a F67S mutation; and
(d) the R171 mutation is a R171K mutation.

6. The mutant rtTA of claim 1, comprising:
(a) G72V;
(b) G12S;
(c) F67S; and
(d) R17K.

7. The mutant rtTA of claim 1, wherein the mutant rtTA comprises a sequence that is at least 70% identical to SEQ ID NO: 13.

8. The mutant rtTA of claim 1, wherein the mutant rtTA comprises a sequence that is at least 80% identical to SEQ ID NO: 13.

9. The mutant rtTA of claim 1, wherein the mutant rtTA comprises a sequence that is at least 90% identical to SEQ ID NO: 13.

10. A polypeptide comprising a sequence that it at least 80% identical to SEQ ID NO: 13 and comprising mutations at residues corresponding to the following positions in rtTA3 (SEQ ID NO: 11):
(a) G72;
(b) G12;
(c) F67; and
(d) R171.

11. A pharmaceutical composition comprising the mutant rtTA of claim 1, and a pharmaceutically acceptable excipient.

12. A kit comprising the mutant rtTA of claim 1.

13. A cell comprising the mutant rtTA of claim 1.

14. The cell of claim 13, wherein the cell is in vitro or ex vivo.

15. The cell of claim 13, wherein the cell is a prokaryotic or eukaryotic cell.

16. The cell of claim 13, wherein the prokaryotic cell is a bacterial or archaeal cell.

17. The cell of claim 15, wherein the eukaryotic cell is an animal, plant, fungi or protoctista cell.

18. The cell of claim 17, wherein the animal cell is a mammalian cell.
